(19) United States
(12) Patent Application Publication
(10) Pub. No.: US 2009/0264411 A1
(43) Pub. Date: Oct. 22, 2009

(54) FUSED THIOPHENE DERIVATIVES AS MEK INHIBITORS

(75) Inventors: Victoria Elizabeth Laing, Slough (GB); Daniel Christopher Brookings, Slough (GB); Rachel Jane Carbery, Trowbridge (GB); Jose Miguel Gascon Simorte, Slough (GB); Martin Clive Hutchings, Slough (GB); Barry John Langham, Slough (GB); Martin Alexander Lowe, Slough (GB)

Correspondence Address: FELDMANGALE, P.A., 1700 Market Street, Suite # 3130, Philadelphia, PA, 19103, US

(73) Assignee: UCB Pharma S.A., Brussels (BE)

(21) Appl. No.: 12/371,124

(22) Filed: Feb. 13, 2009

Related U.S. Application Data
(63) Continuation of application No. PCT/GB2007/003114, filed on Aug. 15, 2007.

(30) Foreign Application Priority Data
Aug. 15, 2006 (GB) 0616214.3

(51) Int. Cl. A61K 31/55; A61K 31/4365; A61K 31/381; C07D 513/04; C07D 333/52

(52) U.S. Cl. 514/212.06; 514/301; 514/443; 540/521; 546/114; 549/49

(57) ABSTRACT

A series of 4,5,6,7-tetrahydrothieno[2,3-c]azepin-8-one derivatives, and analogues thereof, which are substituted in the 2-position by a substituted anilino moiety, being selective inhibitors of human MEK (MAPKK) enzymes, are accordingly of benefit in medicine, for example in the treatment of inflammatory, autoimmune, cardiovascular, proliferative (including oncological) and nociceptive conditions.

### CROSS-REFERENCE TO RELATED APPLICATION

The present application is a continuation of International Application No.: PCT/GB2007/003114, filed Aug. 15, 2007, which claims priority under 119(a-d) to Great Britain Application No. GB 0616214.3, filed Aug. 15, 2006. Each of these applications is hereby incorporated herein by reference in their entireties.

The present invention relates to a class of fused thiophene derivatives and to their use in therapy. More particularly, the invention is concerned with 4,5,6,7-tetrahydro-thieno[2,3-c]azepin-8-one derivatives, and analogues thereof, which are substituted in the 2-position by a substituted anilino moiety. These compounds are selective inhibitors of MEK (MAPKK) enzymes, and are accordingly of benefit as pharmaceutical agents, especially in the treatment of adverse inflammatory, autoimmune, cardiovascular, proliferative (including oncological) and nociceptive conditions.

MEK enzymes are implicated in a variety of physiological and pathological functions that are believed to be operative in a range of human diseases. These functions are summarised in paragraphs [0004] and [0005] of US 2005/0049276 A1.

The compounds of use in the present invention, being potent and selective MEK inhibitors, are therefore beneficial in the treatment and/or prevention of various human ailments. These include autoimmune and inflammatory disorders such as rheumatoid arthritis, osteoarthritis, multiple sclerosis, asthma, inflammatory bowel disease, psoriasis and transplant rejection; cardiovascular disorders including thrombosis, cardiac hypertrophy, hypertension, and irregular contractility of the heart (e.g. during heart failure); proliferative disorders such as restenosis, and oncological conditions including leukaemia, glioblastoma, lymphoma, melanoma, and human cancers of the liver, bone, skin, brain, pancreas, lung, breast, stomach, colon, rectum, prostate, ovary and cervix; and pain and nociceptive disorders, including chronic pain and neuropathic pain.

In addition, the compounds of use in the present invention may be beneficial as pharmacological standards for use in the development of new biological tests and in the search for new pharmacological agents. Thus, the compounds of use in this invention may be useful as radioligands in assays for detecting compounds capable of binding to human MEK enzymes.

WO 2005/023818 describes a broad-ranging class of compounds based on a fused heterobicyclic ring system, which generically encompasses 5,6-dihydro-1-benzothiophen-7(4H)-one and 5,6-dihydrothieno[2,3-c]pyridin-7(4H)-one derivatives attached to a substituted anilino moiety but nowhere specifically discloses any actual compound of this type. Whilst no discrete pharmacological activity is ascribed to the compounds described therein, they are nevertheless stated to be useful inter alia in the treatment of cell proliferative diseases such as cancer.

Nowhere in the prior art, however, is there the precise disclosure of a class of 4,5,6,7-tetrahydrothieno[2,3-c]azepin-8-one derivatives, and analogues thereof, attached at the 2-position to a substituted anilino moiety. It has now been found that such compounds are particularly valuable as selective inhibitors of MEK enzymes.

The compounds of the present invention are potent and selective MEK inhibitors having a binding affinity (IC₅₀) for the human MEK1 and/or MEK2 enzyme of 50 μM or less, generally of 20 μM or less, usually of 5 μM or less, typically of 1 μM or less, suitably of 500 nM or less, ideally of 100 nM or less, and preferably of 20 nM or less (the skilled person will appreciate that a lower IC₅₀ figure denotes a more active compound). The compounds of the invention may possess at least a 10-fold selective affinity, typically at least a 20-fold selective affinity, suitably at least a 50-fold selective affinity, and ideally at least a 100-fold selective affinity, for the human MEK1 and/or MEK2 enzyme relative to other human kinases.

The present invention provides a compound of formula (I), or a pharmaceutically acceptable salt or solvate thereof:

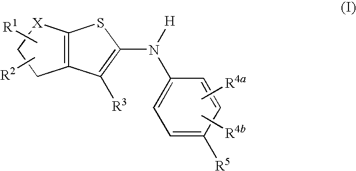

wherein

-X- represents a group of formula (a), (b), (c), (d), (e), (f) or (g):

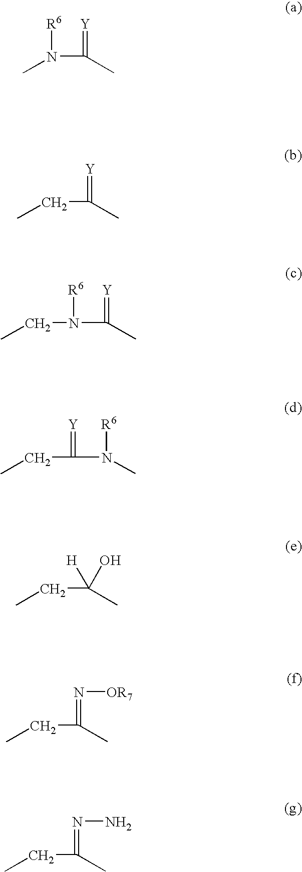

Y represents oxygen, sulphur or N—R⁸;

R¹ and R² independently represent hydrogen; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents; or

R¹ and R², when both are attached to the same carbon atom, represent, when taken together with the carbon atom to which they are both attached, C₃₋₇ cycloalkyl or C₃₋₇ heterocycloalkyl, either of which groups may be optionally substituted by one or more substituents; or

R¹ and R², when attached to adjacent carbon atoms, represent, when taken together with the carbon atoms to which they are attached, C₅₋₇ cycloalkyl, phenyl or heteroaryl, any of which groups may be optionally benzo-fused and/or substituted by one or more substituents;

R³ represents hydrogen, C₁₋₆ alkyl, C₃₋₇ heterocycloalkenyl (optionally substituted by one or two methyl groups), cyano, —CO₂R^{a}, —COR^{b}, —CONR^{b}R^{c}, —SO₂NR^{b}R^{c}, —CON(OR^{b})R^{c}, —CON(R^{c})COR^{b}, —CON(R^{c})SO₂R^{b}, —SO₂N(R^{c})COR^{b}, —CON(R^{d})NR^{b}R^{c}, —C(═NR^{c})NR^{b}R^{c} or —CON(R^{d})C(═NR^{c})NR^{b}R^{c}; or

R³ represents an optionally substituted five-membered heteroaromatic ring selected from furan, thiophene, pyrrole, oxazole, thiazole, isoxazole, isothiazole, imidazole, pyrazole, oxadiazole, thiadiazole, triazole and tetrazole; or

R³ represents an optionally substituted six-membered heteroaromatic ring selected from pyridine, pyrazine, pyrimidine, pyridazine and triazine;

R^{4a} and R^{4b} independently represent hydrogen, halogen, cyano, nitro, C₁₋₆ alkyl, trifluoromethyl, C₁₋₆ alkoxy, trifluoromethoxy, C₁₋₆ alkylthio, C₁₋₆ alkylsulphinyl or C₁₋₆ alkylsulphonyl;

R⁵ represents halogen, nitro, cyano, C₁₋₆ alkyl, C₂₋₆ alkynyl, hydroxy(C₁₋₆)alkyl or formyl;

R⁶ represents hydrogen, C₁₋₆ alkyl, formyl, C₂₋₆ alkylcarbonyl, trifluoromethylcarbonyl or C₁₋₆ alkylsulphonyl;

R⁷ and R⁸ independently represent hydrogen or C₁₋₆ alkyl;

R^{a} represents hydrogen, C₁₋₆ alkyl or C₃₋₇ heterocycloalkyl(C₁₋₆)alkyl;

R^{b} represents hydrogen; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₄₋₉ heterobicycloalkyl, heteroaryl or heteroaryl(C₁₋₆)alkyl, any of which groups may be optionally substituted by one or more substituents; and

R^{c} represents hydrogen or C₁₋₆ alkyl (optionally substituted by hydroxy); or

R^{b} and R^{c}, when taken together with the nitrogen atom to which they are both attached, represent azetidinyl, pyrrolidinyl, piperidinyl, morpholinyl, thiomorpholinyl, piperazinyl, homopiperidinyl, homomorpholinyl or homopiperazinyl, any of which groups may be optionally substituted by one or more substituents; and

R^{d} and R^{e} independently represent hydrogen or C₁₋₆ alkyl.

The present invention also provides a compound of formula (I) as depicted above, or a pharmaceutically acceptable salt or solvate thereof, wherein

R³ represents hydrogen, cyano, —CO₂R^{a}, —COR^{b}, —CONR^{b}R^{c}, —SO₂NR^{b}R^{c}, —CON(OR^{b})R^{c}, —CON(R^{c})COR^{b}, —CON(R^{c})SO₂R^{b}, —SO₂N(R^{c})COR^{b}, —CON(R^{d})NR^{b}R^{c}, —C(═NR^{e})NR^{b}R^{c} or —CON(R^{d})C(═NR^{c})NR^{b}R^{c}; or

R³ represents an optionally substituted five-membered heteroaromatic ring selected from furan, thiophene, pyrrole, oxazole, thiazole, isoxazole, isothiazole, imidazole, pyrazole, oxadiazole, thiadiazole, triazole and tetrazole; or

R³ represents an optionally substituted six-membered heteroaromatic ring selected from pyridine, pyrazine, pyrimidine, pyridazine and triazine;

R⁵ represents halogen, nitro, C₁₋₆ alkyl, hydroxy(C₁₋₆)alkyl or formyl;

R^{a} represents hydrogen or C₁₋₆ alkyl;

R^{b} represents hydrogen; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; and

R^{c} represents hydrogen or C₁₋₆ alkyl; or

R^{b} and R^{c}, when taken together with the nitrogen atom to which they are both attached, represent azetidinyl, pyrrolidinyl, piperidinyl, morpholinyl, thiomorpholinyl, piperazinyl, homopiperidinyl or homomorpholinyl, any of which groups may be optionally substituted by one or more substituents; and

R¹, R², R^{4a}, R^{4b}, R^{d} and R^{c} are as defined above.

Where a group in the compounds of formula (I) above is stated to be optionally substituted, this group may be unsubstituted, or substituted by one or more substituents. Typically, such a group will be unsubstituted, or substituted by one or two substitutents. Suitably, such a group will be unsubstituted or monosubstituted.

For use in medicine, the salts of the compounds of formula (I) will be pharmaceutically acceptable salts. Other salts may, however, be useful in the preparation of the compounds of the invention or of their pharmaceutically acceptable salts. Suitable pharmaceutically acceptable salts of the compounds of this invention include acid addition salts which may, for example, be formed by mixing a solution of the compound of the invention with a solution of a pharmaceutically acceptable acid such as hydrochloric acid, sulphuric acid, methanesulphonic acid, fumaric acid, maleic acid, succinic acid, acetic acid, benzoic acid, citric acid, tartaric acid or phosphoric acid. Furthermore, where the compounds of the invention carry an acidic moiety, e.g. carboxy, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The present invention includes within its scope solvates of the compounds of formula (I) above. Such solvates may be formed with common organic solvents, e.g. hydrocarbon solvents such as benzene or toluene; chlorinated solvents such as chloroform or dichloromethane; alcoholic solvents such as methanol, ethanol or isopropanol; ethereal solvents such as diethyl ether or tetrahydrofuran; or ester solvents such as ethyl acetate. Alternatively, the solvates of the compounds of formula (I) may be formed with water, in which case they will be hydrates.

Suitable alkyl groups which may be present on the compounds of the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl and 2,2-dimethylpropyl. Derived expressions such as “C₁₋₆ alkoxy”, “C₁₋₆ alkylthio”, “C₁₋₆ alkylsulphonyl” and “C₁₋₆ alkylamino” are to be construed accordingly.

Specific C₃₋₇ cycloalkyl groups are cyclopropyl, cyclobutyl, cyclopentyl, cyclohexyl and cycloheptyl.

Suitable aryl groups include phenyl and naphthyl, preferably phenyl.

Suitable aryl(C₁₋₆)alkyl groups include benzyl, phenylethyl, phenylpropyl and naphthylmethyl.

Suitable heterocycloalkyl groups, which may comprise benzo-fused analogues thereof, include azetidinyl, tetrahydrofuranyl, tetrahydrothienyl, pyrrolidinyl, indolinyl, thiazolidinyl, imidazolidinyl, tetrahydropyranyl, piperidinyl, 1,2,3,4-tetrahydroquinolinyl, 1,2,3,4-tetrahydroisoquinolinyl, piperazinyl, 1,2,3,4-tetrahydroquinoxalinyl, morpholinyl, thiomorpholinyl, homopiperidinyl and homopiperazinyl.

A typical C₃₋₇ heterocycloalkenyl group is dihydroimidazolyl (e.g. 4,5-dihydro-1H-imidazol-2-yl).

A typical C₄₋₉ heterobicycloalkyl group is azabicyclo[2.2.2]octyl (e.g. quinuclidin-3-yl).

Suitable heteroaryl groups include furyl, benzofuryl, dibenzofuryl, thienyl, benzothienyl, pyrrolyl, indolyl, pyrrolo[2,3-b]pyridinyl, pyrazolyl, indazolyl, oxazolyl, isoxazolyl, thiazolyl, isothiazolyl, imidazolyl, imidazo[1,2-a]pyridinyl, benzimidazolyl, oxadiazolyl, thiadiazolyl, triazolyl, benzotriazolyl, tetrazolyl, pyridinyl, quinolinyl, isoquinolinyl, pyridazinyl, pyrimidinyl and pyrazinyl groups.

The term “halogen” as used herein is intended to include fluorine, chlorine, bromine and iodine atoms.

Where the compounds of formula (I) have one or more asymmetric centres, they may accordingly exist as enantiomers. Where the compounds of the invention possess two or more asymmetric centres, they may additionally exist as diastereomers. The invention is to be understood to extend to all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)-enol (CH═CHOH) tautomers. Formula (I) and the formulae depicted hereinafter are intended to represent all individual tautomers and all possible mixtures thereof, unless stated or shown otherwise.

Specific sub-classes of compounds in accordance with the present invention are represented by the compounds of formula (IA), (IB), (IC), (ID), (IE), (IF) and (IG):

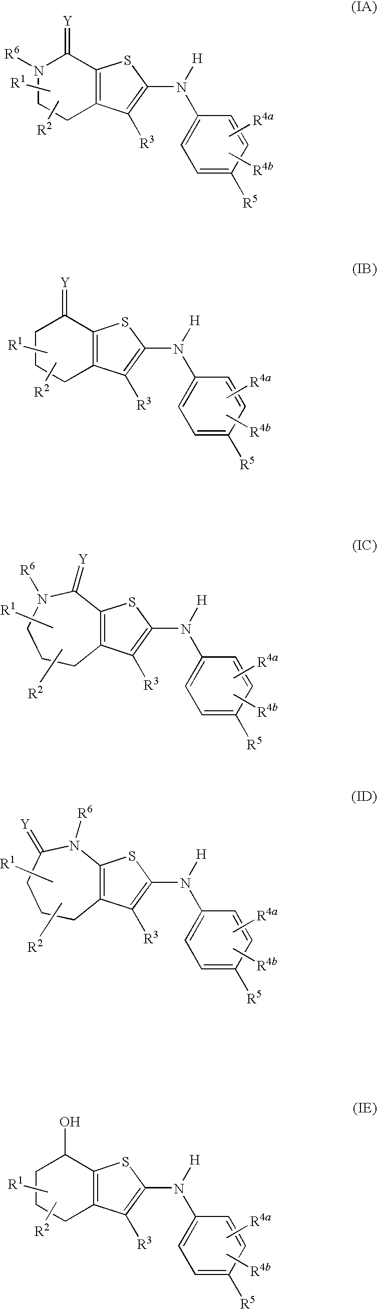

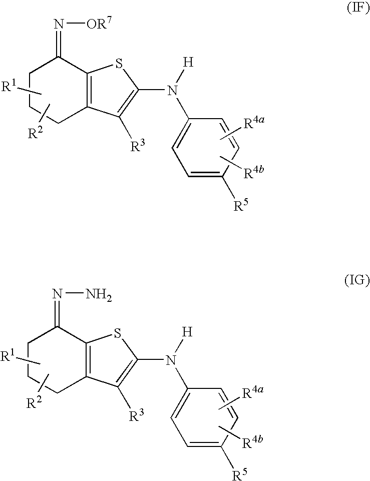

wherein Y, R¹, R², R³, R^{4a}, R^{4b}, R⁵, R⁶ and R⁷ are as defined above.

Selected sub-classes of compounds in accordance with the present invention are represented by the compounds of formula (IA), (IB) and (IC) as depicted above. In one embodiment, the compounds according to the present invention are represented by formula (IA) as depicted above. In another embodiment, the compounds according to the present invention are represented by formula (IB) as depicted above. In a further embodiment, the compounds according to the present invention are represented by formula (IC) as depicted above.

A particular sub-class of compounds in accordance with the present invention is represented by the compounds of formula (IC) as depicted above.

In the compounds of formula (I), -X- typically represents a group of formula (a), (b) or (c) as depicted above. In one embodiment, -X- represents a group of formula (a) as depicted above. In another embodiment, -X- represents a group of formula (b) as depicted above. In a further embodiment, -X- represents a group of formula (c) as depicted above.

In the compounds of formula (I), -X- suitably represents a group of formula (c) as depicted above.

In one embodiment, Y is oxygen. In another embodiment, Y is sulphur. In a further embodiment, Y is N—R⁸ in which R⁸ is as defined above.

Suitably, R¹ represents hydrogen; or C₁₋₆ alkyl, aryl or heteroaryl, any of which groups may be optionally substituted by one or more substituents.

Suitably, R² represents hydrogen or optionally substituted C₁₋₆ alkyl.

Examples of typical substituents on R¹ and/or R² include halogen, cyano, nitro, C₁₋₆ alkyl, trifluoromethyl, hydroxy, C₁₋₆ alkoxy, difluoromethoxy, trifluoromethoxy, aryloxy, C₁₋₆ alkylthio, C₁₋₆ alkylsulphonyl, amino, C₁₋₆ alkylamino, di(C₁₋₆)alkylamino, C₂₋₆ alkylcarbonylamino, C₂₋₆ alkoxycarbonylamino, C₁₋₆ alkylsulphonylamino, formyl, C₂₋₆ alkylcarbonyl, carboxy, C₂₋₆ alkoxycarbonyl, aminocarbonyl, C₁₋₆ alkylamino-carbonyl, di(C₁₋₆)alkylaminocarbonyl, aminosulphonyl, C₁₋₆ alkylaminosulphonyl and di(C₁₋₆)alkylaminosulphonyl; especially halogen, C₁₋₆ alkoxy or C₁₋₆ alkylthio.

Examples of particular substituents on R¹ and/or R² include fluoro, chloro, bromo, cyano, nitro, methyl, trifluoromethyl, hydroxy, methoxy, difluoromethoxy, trifluoro-methoxy, phenoxy, methylthio, methylsulphonyl, amino, methylamino, dimethylamino, acetylamino, methoxycarbonylamino, methylsulphonylamino, formyl, acetyl, carboxy, methoxycarbonyl, aminocarbonyl, methylaminocarbonyl, dimethylaminocarbonyl, aminosulphonyl, methylaminosulphonyl and dimethylaminosulphonyl; especially chloro, methoxy or methylthio.

Typical values of R¹ include hydrogen, methyl, n-propyl, isopropyl, phenyl, chlorophenyl, methoxyphenyl, methylthiophenyl and furyl. In one embodiment, R¹ is hydrogen. A particular value of R¹ is methyl.

Typical values of R² include hydrogen and methyl. In one embodiment, R² is hydrogen. In another embodiment, R² is C₁₋₆ alkyl, especially methyl.

Alternatively, R¹ and R², when both are attached to the same carbon atom, may together form an optionally substituted spiro linkage. Thus, R¹ and R², when both are attached to the same carbon atom, may represent, when taken together with the carbon atom to which they are both attached, C₃₋₇ cycloalkyl or C₃₋₇ heterocycloalkyl, either of which groups may be unsubstituted, or substituted by one or more, typically by one or two, substituents. In this context, R¹ and R², when taken together with the carbon atom to which they are both attached, may suitably represent an optionally substituted cyclopentyl, cyclohexyl, pyrrolidine or piperidine ring, especially cyclopentyl or cyclohexyl.

Alternatively, R¹ and R², when attached to adjacent carbon atoms, may together form an optionally benzo-fused and/or substituted cycloalkyl, phenyl or heteroaryl (e.g. pyridinyl) ring fused to the ring containing the variable X. Thus, R¹ and R², when attached to adjacent carbon atoms, may represent, when taken together with the carbon atoms to which they are attached, C₅₋₇ cycloalkyl, phenyl or heteroaryl (e.g. pyridinyl), any of which groups may be benzo-fused and/or unsubstituted, or substituted by one or more, typically by one or two, substituents. In this context, in one embodiment, R¹ and R², when taken together with the adjacent carbon atoms to which they are attached, suitably represent a phenyl ring fused to the ring containing the variable X. Also in this context, in another embodiment, R¹ and R², when taken together with the adjacent carbon atoms to which they are attached, suitably represent a benzo-fused cyclopentyl ring, i.e. an indanyl moiety fused to the ring containing the variable X.

Typically, R^{a} represents hydrogen or C₁₋₆ alkyl. Suitably, R^{a} represents hydrogen, methyl or ethyl, especially hydrogen or ethyl. In one embodiment, R^{a} represents hydrogen. In another embodiment, R^{a} represents methyl. In a further embodiment, R^{a} represents ethyl. In a still further embodiment, R^{a} represents C₃₋₇ heterocycloalkyl-(C₁₋₆)alkyl, especially piperidinylmethyl.

In a favoured embodiment, R^{b} represents hydrogen; or C₁₋₆ alkyl, C₃₋₇ cycloalkyl-(C₁₋₆)alkyl, aryl(C₁₋₆)alkyl, C₃₋₇ heterocycloalkyl, C₃₋₇ heterocycloalkyl(C₁₋₆)alkyl, C₄₋₉ heterobicycloalkyl, heteroaryl or heteroaryl(C₁₋₆)alkyl, any of which groups may be optionally substituted by one or more substituents.

In an illustrative embodiment, R^{b} represents hydrogen; or C₁₋₆ alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents.

Suitably, R^{b} represents hydrogen; or C₁₋₆ alkyl, C₃₋₇ heterocycloalkyl, C₃₋₇ heterocycloalkyl(C₁₋₆)alkyl or heteroaryl(C₁₋₆)alkyl, any of which groups may be optionally substituted by one or more substituents.

In a definitive embodiment, R^{b} represents hydrogen; or methyl, ethyl, propyl, butyl, pentyl, hexyl, cyclohexylmethyl, benzyl, phenylethyl, azetidinyl, tetrahydrofuryl, tetrahydrothienyl, pyrrolidinyl, piperidinyl, homopiperidinyl, quinuclidinyl, azetidinylmethyl, tetrahydrofurylmethyl, pyrrolidinylmethyl, pyrrolidinylethyl, pyrrolidinylpropyl, thiazolidinylmethyl, imidazolidinylethyl, piperidinylmethyl, piperidinylethyl, tetrahydroquinolinylmethyl, piperazinylpropyl, morpholinylethyl, morpholinylpropyl, pyridinyl, indolylethyl, pyrazolylethyl, imidazolylmethyl, imidazolylethyl, benzimidazolylmethyl, triazolylethyl, pyridinylmethyl or pyridinylethyl, any of which groups may be optionally substituted by one or more substituents.

Definitive examples of suitable substituents on R^{b}, or on the cyclic moiety —NR^{b}R^{c}, include C₁₋₆ alkyl, C₁₋₆ alkoxy, C₁₋₆ alkylamino(C₁₋₆)alkoxy, C₁₋₆ alkoxy(C₁₋₆)-alkyl, C₁₋₆ alkylthio, C₁₋₆ alkylsulphonyl, hydroxy, hydroxy(C₁₋₆)alkyl, amino(C₁₋₆)alkyl, nitro(C₁₋₆)alkyl, cyano, trifluoromethyl, oxo, C₂₋₆ alkylcarbonyl, carboxy, C₂₋₆ alkoxycarbonyl, amino, C₁₋₆ alkylamino, di(C₁₋₆)alkylamino, bis[hydroxy(C₁₋₆)alkyl]-amino, C₁₋₆ alkylamino(C₁₋₆)alkylamino, phenylamino, pyridinylamino, C₂₋₆ alkylcarbonylamino, C₂₋₆ alkoxycarbonylamino, [(C₂₋₆)alkoxycarbonyl][(C₁₋₆)alkyl]-amino, bis[(C₂₋₆)alkoxycarbonyl(C₁₋₆)alkyl]amino, C₂₋₆ alkoxycarbonylamino(C₁₋₆)alkyl, aminocarbonyl and guanidinyl.

Examples of typical substituents on R^{b}, or on the cyclic moiety —NR^{b}R^{c}, include C₁₋₆ alkyl, C₁₋₆ alkoxy, hydroxy, hydroxy(C₁₋₆)alkyl, amino(C₁₋₆)alkyl, (amino)(hydroxy)-(C₁₋₆)alkyl, halogen, oxo, C₂₋₆ alkylcarbonyl, carboxy, C₂₋₆ alkoxycarbonyl, di(C₁₋₆)alkylhydrazinylcarbonyl, amino, C₁₋₆ alkylamino, di(C₁₋₆)alkylamino, C₂₋₆ alkylcarbonylamino, aminocarbonylamino, aminocarbonyl, C₁₋₆ alkylaminocarbonyl, di(C₁₋₆)alkylaminocarbonyl, aminosulfonyl, C₁₋₆ alkylsulfonyl and C₁₋₆ alkylaminocarbonyl(C₁₋₆)alkyl; especially C₁₋₆ alkyl, hydroxy, hydroxy(C₁₋₆)alkyl or amino.

Definitive examples of specific substituents on R^{b}, or on the cyclic moiety —NR^{b}R^{c}, include methyl, ethyl, isopropyl, methoxy, isopropoxy, methylaminoethoxy, methoxymethyl, methylthio, ethylthio, methylsulphonyl, hydroxy, hydroxymethyl, hydroxyethyl, aminomethyl, nitromethyl, cyano, trifluoromethyl, oxo, acetyl, carboxy, methoxycarbonyl, ethoxycarbonyl, tert-butoxycarbonyl, amino, methylamino, ethylamino, dimethylamino, bis[hydroxyethyl]amino, ethylaminoethylamino, phenylamino, pyridinylamino, acetylamino, tert-butoxycarbonylamino, (tert-butoxy-carbonyl)(methyl)amino, bis(ethoxycarbonylmethyl)amino, tert-butoxycarbonylamino-methyl, aminocarbonyl and guanidinyl.

Examples of particular substituents on R^{b}, or on the cyclic moiety —NR^{c}R^{c}, include methyl, methoxy, hydroxy, hydroxymethyl, 2-hydroxyethyl, aminomethyl, 2-amino-3-hydroxypropyl, fluoro, oxo, acetyl, carboxy, methoxycarbonyl, ethoxycarbonyl, tert-butoxycarbonyl, dimethylhydrazinylcarbonyl, amino, methylamino, 1,3-dimethylbutylamino, dimethylamino, acetylamino, aminocarbonylamino, aminocarbonyl, ethylaminocarbonyl, diethylaminocarbonyl, aminosulfonyl, methylsulfonyl and methylaminocarbonylmethyl; especially methyl, hydroxy, hydroxymethyl or amino.

Typically, R^{b} represents C₁₋₆ alkyl, optionally substituted by one or more, preferably one or two, hydroxy groups.

Specific values of R^{b} include hydrogen, methyl, carboxymethyl, aminocarbonyl-methyl, methoxyethyl (especially 2-methoxyethyl), methylaminoethoxyethyl (especially 2-[2-(methylamino)ethoxy]ethyl), ethylthioethyl (especially 2-(ethylthio)ethyl), methylsulphonylethyl (especially 2-(methylsulphonyl)ethyl), hydroxyethyl (especially 2-hydroxyethyl), cyanoethyl (especially 2-cyanoethyl), (hydroxy)(trifluoromethyl)ethyl (especially 2-hydroxy-3,3,3-trifluoropropyl), carboxyethyl (especially 2-carboxyethyl), ethoxycarbonylethyl (especially 2-(ethoxycarbonyl)ethyl), aminoethyl (especially 2-aminoethyl), (amino)(carboxy)ethyl (especially 2-amino-2-carboxyethyl), methylamino-ethyl (especially 2-(methylamino)ethyl), dimethylaminoethyl (especially 2-(dimethylamino)ethyl), bis(hydroxyethyl)aminoethyl (especially 2-[bis(2-hydroxyethyl)amino]-ethyl), ethylaminoethylaminoethyl (especially 2-[2-(ethylamino)ethylamino]ethyl), phenylaminoethyl (especially 2-(phenylamino)ethyl), pyridinylaminoethyl (especially 2-(pyridin-2-ylamino)ethyl), acetylaminoethyl (especially 2-(acetylamino)ethyl), (tert-butoxycarbonylamino)(carboxy)ethyl (especially 2-(tert-butoxycarbonylamino)-2-carboxyethyl), (tert-butoxycarbonyl)(methyl)aminoethyl (especially 2-[N-(tert-butoxycarbonyl)-N-methylamino]ethyl), aminocarbonylethyl (especially 1-(aminocarbonyl)ethyl), propyl, methoxypropyl (especially 2-methoxy-1-methylethyl), isopropoxypropyl (especially 3-isopropoxypropyl), hydroxypropyl (especially 2-hydroxypropyl or 3-hydroxypropyl), dihydroxypropyl (especially 2,3-dihydroxypropyl), (carboxy)(methylthio)propyl (especially 1-carboxy-3-(methylthio)propyl), ethoxycarbonylpropyl (especially 2-ethoxycarbonyl-1-methylethyl), aminopropyl (especially 3-aminopropyl), (amino)(hydroxy)propyl (especially 3-amino-2-hydroxypropyl), methylaminopropyl (especially 3-(methylamino)propyl), ethylaminopropyl (especially 3-(ethylamino)propyl), dimethylaminopropyl (especially 3-(dimethylamino)propyl), tert-butyl, hydroxybutyl (especially 1,1-dimethyl-2-hydroxyethyl or 2-hydroxy-2-methylpropyl), dihydroxybutyl (especially 3,4-dihydroxybutyl), aminobutyl (especially 2-amino-2-methylpropyl or 4-aminobutyl), (carboxy)(guanidinyl)butyl (especially 1-carboxy-4-(guanidinyl)butyl), aminopentyl (especially 3-amino-2,2-dimethylpropyl), dimethylaminopentyl (especially 3-(dimethylamino)-2,2-dimethylpropyl), hydroxyhexyl (especially 1-(tert-butyl)-2-hydroxyethyl), hydroxycyclohexylmethyl, (aminomethyl)-cyclohexylmethyl, methoxybenzyl, (hydroxy)(phenyl)ethyl, (oxo)(phenyl)ethyl, (carboxy)(hydroxyphenyl)ethyl, azetidinyl, (oxo)tetrahydrofuryl, (dioxo)tetrahydrothienyl, pyrrolidinyl, methylpyrrolidinyl, tert-butoxycarbonylpyrrolidinyl, piperidinyl, methylpiperidinyl, (oxo)homopiperidinyl, quinuclidinyl, azetidinylmethyl, hydroxyazetidinylmethyl, tert-butoxycarbonylazetidinylmethyl, (tert-butoxycarbonyl)(hydroxy)-azetidinylmethyl, tetrahydrofurylmethyl, pyrrolidinylmethyl, ethylpyrrolidinylmethyl, pyrrolidinylethyl, methylpyrrolidinylethyl, (carboxy)(isopropyl)(oxo)pyrrolidinylethyl, (oxo)pyrrolidinylpropyl, thiazolidinylmethyl, (oxo)imidazolidinylethyl, piperidinylmethyl, methylpiperidinylmethyl, tert-butoxycarbonylpiperidinylmethyl, piperidinylethyl, tetrahydroquinolinylmethyl, methylpiperazinylpropyl, morpholinylethyl, morpholinylpropyl, pyridinyl, aminopyridinyl, (carboxy)indolylethyl, dimethylpyrazolylethyl, methylimidazolylmethyl, imidazolylethyl, benzimidazolylmethyl, triazolylethyl, pyridinylmethyl and pyridinylethyl.

Typical values of R^{b} include hydrogen, methyl, aminoethyl (especially 2-aminoethyl), hydroxypropyl (especially 2-hydroxypropyl or 3-hydroxypropyl), aminopropyl (especially 3-aminopropyl), dihydroxypropyl (especially 2,3-dihydroxypropyl), (amino)(hydroxy)propyl (especially 3-amino-2-hydroxypropyl), hydroxybutyl (especially 1,1-dimethyl-2-hydroxyethyl or 2-hydroxy-2-methylpropyl), aminobutyl (especially 2-amino-2-methylpropyl or 4-aminobutyl), dihydroxybutyl (especially 3,4-dihydroxybutyl), piperidinyl (especially piperidin-3-yl), methylpiperidinyl (especially 1-methylpiperidin-4-yl), piperidinylmethyl (especially piperidin-4-ylmethyl), imidazolylethyl [especially 2-(imidazol-5-yl)ethyl] and pyridinylethyl [especially 2-(pyridin-3-yl)ethyl or 2-(pyridin-4-yl)ethyl].

Suitably, R^{c} represents hydrogen or C₁₋₆ alkyl. In one embodiment, R^{c} is hydrogen. In another embodiment, R^{c} represents C₁₋₆ alkyl, especially methyl or ethyl, particularly methyl. In a further embodiment, R^{c} represents hydroxy-substituted C₁₋₆ alkyl, e.g. hydroxyethyl (especially 2-hydroxyethyl).

Alternatively, the moiety —NR^{b}R^{c} may suitably represent azetidin-1-yl, pyrrolidin-1-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents. Favourably, the moiety —NR^{b}R^{c} may suitably represent azetidin-1-yl, pyrrolidin-1-yl, piperidin-1-yl, morpholin-4-yl, piperazin-1-yl or homopiperazin-1-yl, any of which groups may be optionally substituted by one or more substituents. Typically, the moiety —NR^{b}R^{c} may suitably represent azetidin-1-yl, pyrrolidin-1-yl or piperazin-1-yl, any of which groups may be optionally substituted by one or more substituents.

Typical substituents on the cyclic moiety —NR^{b}R^{c} include C₁₋₆ alkyl, C₁₋₆ alkoxy(C₁₋₆)alkyl, hydroxy, hydroxy(C₁₋₆)alkyl, amino(C₁₋₆)alkyl, nitro(C₁₋₆)alkyl, oxo, C₂₋₆ alkylcarbonyl, carboxy, C₂₋₆ alkoxycarbonyl, amino, C₂₋₆ alkylcarbonylamino, C₂₋₆ alkoxycarbonylamino, bis[(C₂₋₆)alkoxycarbonyl(C₁₋₆)alkyl]amino, C₂₋₆ alkoxycarbonylamino(C₁₋₆)alkyl and aminocarbonyl. Specific substituents include methyl, ethyl, methoxymethyl, hydroxy, hydroxymethyl, hydroxyethyl, aminomethyl, nitromethyl, oxo, acetyl, carboxy, methoxycarbonyl, tert-butoxycarbonyl, amino, acetylamino, tert-butoxycarbonylamino, bis(ethoxycarbonylmethyl)amino, tert-butoxycarbonylaminomethyl and aminocarbonyl.

Suitably, the cyclic moiety —NR^{b}R^{c} may be substituted by C₁₋₆ alkyl, hydroxy or hydroxy(C₁₋₆)alkyl; especially methyl, hydroxy or hydroxymethyl.

Definitive values of —NR^{b}R^{c} include hydroxyazetidin-1-yl, (hydroxy)(nitro-methyl)azetidin-1-yl, aminoazetidin-1-yl, (aminomethyl)azetidin-1-yl, (aminomethyl)-(hydroxy)azetidin-1-yl, (tert-butoxycarbonylaminomethyl)azetidin-1-yl, pyrrolidin-1-yl, (methoxymethyl)pyrrolidin-1-yl, hydroxypyrrolidin-1-yl, (hydroxymethyl)pyrrolidin-1-yl, (aminomethyl)pyrrolidin-1-yl, carboxypyrrolidin-1-yl, (methoxycarbonyl)pyrrolidin-1-yl, aminopyrrolidin-1-yl, (acetylamino)pyrrolidin-1-yl, (tert-butoxycarbonylamino)-pyrrolidin-1-yl, [bis(ethoxycarbonylmethyl)amino]pyrrolidin-1-yl, (tert-butoxycarbonyl-aminomethyl)pyrrolidin-1-yl, hydroxypiperidin-1-yl, (hydroxymethyl)piperidin-1-yl, (hydroxyethyl)piperidin-1-yl, carboxypiperidin-1-yl, (aminocarbonyl)piperidin-1-yl, piperazin-1-yl, methylpiperazin-1-yl, ethylpiperazin-1-yl, (hydroxyethyl)piperazin-1-yl, oxopiperazin-1-yl, acetylpiperazin-1-yl, carboxypiperazin-1-yl, (tert-butoxycarbonyl)-(carboxy)piperazin-1-yl, morpholin-4-yl, dimethylmorpholin-4-yl, (hydroxymethyl)-morpholin-4-yl and homopiperazin-1-yl.

Particular values of —NR^{b}R^{c} include 3-hydroxyazetidin-1-yl, pyrrolidin-1-yl, 3-hydroxypyrrolidin-1-yl, 2-(hydroxymethyl)pyrrolidin-1-yl, piperazin-1-yl and 4-methylpiperazin-1-yl.

In one embodiment, R^{d} is hydrogen. In another embodiment, R^{d} represents C₁₋₆ alkyl, especially methyl.

In one embodiment, R^{e} is hydrogen. In another embodiment, R^{e} represents C₁₋₆ alkyl, especially methyl.

Where R³ represents a five-membered heteroaromatic ring, this ring may be optionally substituted by one or, where possible, two substituents. As will be appreciated, where R³ represents an oxadiazole, thiadiazole or tetrazole ring, only one substituent will be possible; otherwise, one or two optional substituents may be accommodated around the five-membered heteroaromatic ring R³. Where R³ represents a six-membered heteroaromatic ring, this ring may be optionally substituted by one or more substituents, typically by one or two substituents. Examples of suitable substituents on the five-membered or six-membered heteroaromatic ring as specified for R³ include C₁₋₆ alkyl, C₃₋₇ cycloalkyl, aryl, aryl(C₁₋₆)alkyl, C₃₋₇ heterocycloalkyl, heteroaryl, heteroaryl(C₁₋₆)-alkyl, C₁₋₆ alkoxy, C₁₋₆ alkylthio, amino, C₁₋₆ alkylamino, di(C₁₋₆)alkylamino, halogen, cyano and trifluoromethyl.

Favourably, R³ represents hydrogen, C₁₋₆ alkyl, C₃₋₇ heterocycloalkenyl (optionally substituted by one or two methyl groups), cyano, —CO₂R^{a}, —CONR^{b}R^{c}, —CON(OR^{b})R^{c}, —CON(R^{d})NR^{b}R^{c}, —C(═NR^{e})NR^{b}R^{c} or —CON(R^{d})C(═NR^{e})NR^{b}R^{c}, in which R^{a}, R^{b}, R^{c}, R^{d} and R^{e} are as defined above.

Suitably, R³ represents hydrogen, —CO₂R^{a}, —CONR^{b}R^{c}, —CON(OR^{b})R^{c} or —CONHNR^{b}R^{c}, in which R^{a}, R^{b} and R^{c} are as defined above.

In one embodiment, R³ represents hydrogen. In another embodiment, R³ represents C₁₋₆ alkyl, especially methyl. In another embodiment, R³ represents C₃₋₇ heterocycloalkenyl (optionally substituted by one or two methyl groups), e.g. 4,4-dimethyl-4,5-dihydro-1H-imidazol-2-yl. In another embodiment, R³ represents cyano. In another embodiment, R³ represents —CO₂R^{a}, in which R^{a} is as defined above. In a further embodiment, R³ represents —CONR^{b}R^{c}, in which R^{b} and R^{c} are as defined above. In a still further embodiment, R³ represents —CON(OR^{b})R^{c}, in which R^{b} and R^{c} are as defined above. In an additional embodiment, R³ represents —CONHNR^{b}R^{c}, in which R^{b} and R^{c} are as defined above. In another additional embodiment, R³ represents —C(═NH)NR^{b}R^{c}, in which R^{b} and R^{c} are as defined above. In a further additional embodiment, R³ represents —CONHC(═NH)NR^{b}R^{c}, in which R^{b} and R^{c} are as defined above.

Suitable values of R^{4a} and/or R^{4b} include hydrogen, halogen (especially fluoro or chloro) and C₁₋₆ alkyl (especially methyl).

Preferably, R^{4a} is attached at the 2-position relative to the anilino nitrogen atom.

Suitably, R^{4a} represents halogen. In one embodiment, R^{4a} is fluoro. In another embodiment, R^{4a} is chloro.

Typically, R^{4b} may be attached at the 6-position relative to the anilino nitrogen atom.

Suitably, R^{4b} is hydrogen.

Suitably, R⁵ represents halogen, nitro, cyano, C₂₋₆ alkynyl, hydroxy(C₁₋₆)alkyl or formyl. Typically, R⁵ represents halogen, nitro, hydroxy(C₁₋₆)alkyl or formyl.

In one embodiment, R⁵ represents halogen, especially bromo or iodo, particularly iodo. In another embodiment, R⁵ represents nitro. In another embodiment, R⁵ represents cyano. In another embodiment, R⁵ represents C₁₋₆ alkyl, especially methyl. In another embodiment, R⁵ represents C₂₋₆ alkynyl, especially ethynyl. In a further embodiment, R⁵ represents hydroxy(C₁₋₆)alkyl, especially hydroxymethyl. In an additional embodiment, R⁵ represents formyl.

Suitably, R⁶ represents hydrogen or C₁₋₆ alkyl. In one embodiment, R⁶ represents hydrogen. In another embodiment, R⁶ represents C₁₋₆ alkyl, especially methyl.

In one embodiment, R⁷ represents hydrogen. In another embodiment, R⁷ represents C₁₋₆ alkyl, especially methyl.

In one embodiment, R⁸ represents hydrogen. In another embodiment, R⁸ represents C₁₋₆ alkyl, especially methyl.

One sub-class of compounds according to the invention is represented by the compounds of formula (IIA), and pharmaceutically acceptable salts and solvates thereof:

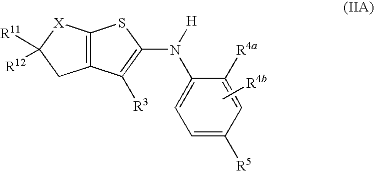

wherein

-X-, R³, R^{4a}, R^{4b} and R⁵ are as defined above;

R¹¹ represents hydrogen or optionally substituted C₁₋₆ alkyl; and

R¹² represents hydrogen; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; or

R¹¹ and R¹², when taken together with the carbon atom to which they are both attached, represent C₃₋₇ cycloalkyl or C₃₋₇ heterocycloalkyl, either of which groups may be optionally substituted by one or more substituents.

Where R¹¹ and/or R¹² in the compounds of formula (IIA) above is stated to be optionally substituted, this group may be unsubstituted, or substituted by one or more substituents. Typically, R¹¹ and/or R¹² will be unsubstituted, or substituted by one or two substitutents. Suitably, R¹¹ and/or R¹² will be unsubstituted or monosubstituted.

Suitably, R¹¹ represents hydrogen or unsubstituted C₁₋₆ alkyl.

Suitably, R¹² represents hydrogen; or C₁₋₆ alkyl, aryl or heteroaryl, any of which groups may be optionally substituted by one or more substituents. Particular values of R¹² include hydrogen and unsubstituted C₁₋₆ alkyl.

Examples of typical substituents on R¹¹ and/or R¹² include halogen, cyano, nitro, C₁₋₆ alkyl, trifluoromethyl, hydroxy, C₁₋₆ alkoxy, difluoromethoxy, trifluoromethoxy, aryloxy, C₁₋₆ alkylthio, C₁₋₆ alkylsulphonyl, amino, C₁₋₆ alkylamino, di(C₁₋₆)alkylamino, C₂₋₆ alkylcarbonylamino, C₂₋₆ alkoxycarbonylamino, C₁₋₆ alkylsulphonylamino, formyl, C₂₋₆ alkylcarbonyl, carboxy, C₂₋₆ alkoxycarbonyl, aminocarbonyl, C₁₋₆ alkylamino-carbonyl, di(C₁₋₆)alkylaminocarbonyl, aminosulphonyl, C₁₋₆ alkylaminosulphonyl and di(C₁₋₆)alkylaminosulphonyl; especially halogen, C₁₋₆ alkoxy or C₁₋₆ alkylthio.

Examples of particular substituents on R¹¹ and/or R¹² include fluoro, chloro, bromo, cyano, nitro, methyl, trifluoromethyl, hydroxy, methoxy, difluoromethoxy, trifluoromethoxy, phenoxy, methylthio, methylsulphonyl, amino, methylamino, dimethylamino, acetylamino, methoxycarbonylamino, methylsulphonylamino, formyl, acetyl, carboxy, methoxycarbonyl, aminocarbonyl, methylaminocarbonyl, dimethylaminocarbonyl, aminosulphonyl, methylaminosulphonyl and dimethylaminosulphonyl; especially chloro, methoxy or methylthio.

Typical values of R¹¹ include hydrogen and methyl. In one embodiment, R¹¹ is hydrogen. In another embodiment, R¹¹ is methyl.

Typical values of R¹² include hydrogen, methyl, n-propyl, isopropyl, phenyl, chlorophenyl, methoxyphenyl, methylthiophenyl and furyl, especially hydrogen or methyl. In one embodiment, R¹² is hydrogen. In another embodiment, R¹² is methyl.

Alternatively, R¹¹ and R¹² may together form an optionally substituted spiro linkage. Thus, R¹¹ and R¹², when taken together with the carbon atom to which they are both attached, may represent C₃₋₇ cycloalkyl or C₃₋₇ heterocycloalkyl, either of which groups may be unsubstituted, or substituted by one or more, typically by one or two, substituents. In this context, R¹¹ and R¹², when taken together with the carbon atom to which they are both attached, may suitably represent an optionally substituted cyclopentyl, cyclohexyl, pyrrolidine or piperidine ring, especially cyclopentyl or cyclohexyl.

One particular sub-group of the compounds of formula (IIA) is represented by the compounds of formula (IIB), and pharmaceutically acceptable salts and solvates thereof:

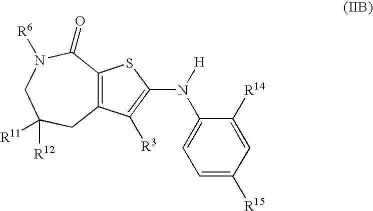

wherein

R³, R⁶, R¹¹ and R¹² are as defined above;

R¹⁴ represents halogen; and

R¹⁵ represents halogen, nitro, cyano, C₂₋₆ alkynyl, hydroxy(C₁₋₆)alkyl or formyl.

In one specific embodiment, R¹⁴ is fluoro. In another specific embodiment, R¹⁴ is chloro.

Typically, R¹⁵ represents halogen, nitro, hydroxy(C₁₋₆)alkyl or formyl.

In one embodiment, R¹⁵ represents halogen, especially iodo. In another embodiment, R¹⁵ represents nitro. In another embodiment, R¹⁵ represents cyano. In another embodiment, R¹⁵ represents C₂₋₆ alkynyl, especially ethynyl. In a further embodiment, R¹⁵ represents hydroxy(C₁₋₆)alkyl, especially hydroxymethyl. In an additional embodiment, R¹⁵ represents formyl.

Another sub-group of the compounds of formula (IIA) is represented by the compounds of formula (IIC), and pharmaceutically acceptable salts and solvates thereof:

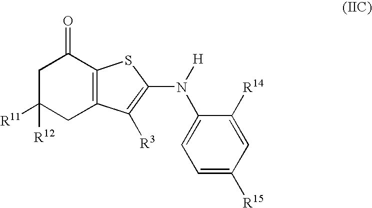

wherein

R³, R¹¹ and R¹², R¹⁴ and R¹⁵ are as defined above.

A further sub-group of the compounds of formula (IIA) is represented by the compounds of formula (IID), and pharmaceutically acceptable salts and solvates thereof:

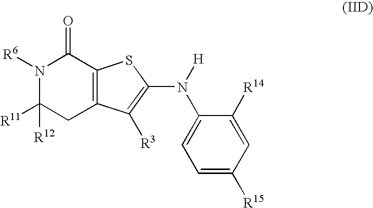

wherein

R³, R⁶, R¹¹, R¹², R¹⁴ and R¹⁵ are as defined above.

Specific novel compounds in accordance with the present invention include each of the compounds whose preparation is described in the accompanying Examples, and pharmaceutically acceptable salts and solvates thereof.

The present invention also provides a pharmaceutical composition which comprises a compound of formula (I) as defined above, or a pharmaceutically acceptable salt or solvate thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds according to the present invention may be conveniently formulated as microionized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds according to the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound of the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

The compounds of formula (I) above may be prepared by a process which comprises reacting a compound of formula (III) with a compound of formula (IV):

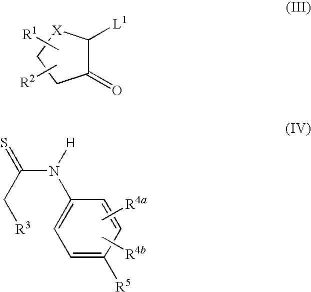

wherein R¹, R², R³, R^{4a}, R^{4b}, R⁵ and X are as defined above, and L¹ represents a suitable leaving group.

The leaving group L¹ is typically a halogen atom, e.g. bromo.

The reaction is conveniently effected at an elevated temperature in a suitable solvent, e.g. N,N-dimethylformamide, typically under basic conditions, e.g. in the presence of a base such as cesium carbonate.

By way of example, the intermediates of formula (IV) above wherein R³ is ethoxycarbonyl may be prepared by a process which comprises reacting the product formed by reacting ethyl acetoacetate and sodium ethoxide with a compound of formula (V):

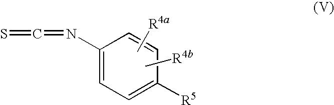

wherein R^{4a}, R^{4b} and R⁵ are as defined above.

The reaction is conveniently effected by stirring the reactants in a suitable solvent, e.g. a lower alkanol such as ethanol.

Similarly, the intermediates of formula (IV) above wherein R³ is cyano may be prepared by a process which comprises reacting compound (V) with acetonitrile, typically in the presence of a strong base such as sodium hexamethyldisilazide.

The intermediates of formula (V) above may be prepared by reacting a compound of formula (VI):

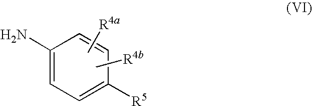

wherein R^{4a}, R^{4b} and R⁵ are as defined above; with thiophosgene.

The reaction is conveniently effected in a suitable solvent, typically a mixture of chloroform and water.

In an alternative procedure, the compounds of formula (I) above may be prepared by a process which comprises reacting a compound of formula (VII) with a compound of formula (VIII):

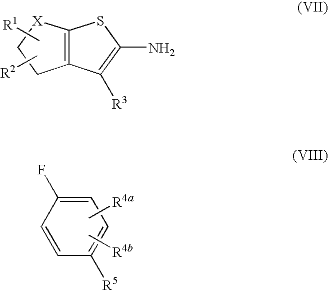

wherein R¹, R², R³, R^{4a}, R^{4b}, R⁵ and X are as defined above.

The reaction is conveniently effected, at an elevated temperature if required, in a suitable solvent, e.g. N,N-dimethylformamide, typically under basic conditions, e.g. in the presence of a base such as cesium carbonate.

By way of example, the intermediates of formula (VII) above wherein R³ represents —CO₂R^{a} may be prepared by reacting a compound of formula N≡C—CH₂—CO₂R^{a} with the appropriate compound of formula (IX):

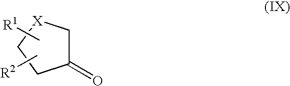

wherein X, R¹, R² and R^{a} are as defined above; in the presence of sulphur.

The reaction is conveniently effected at an elevated temperature in a suitable solvent, e.g. a lower alkanol such as ethanol, typically under basic conditions, e.g. in the presence of morpholine.

In another procedure, the compounds of formula (I) above may be prepared by a process which comprises reacting a compound of formula (VI) as defined above with a compound of formula (X):

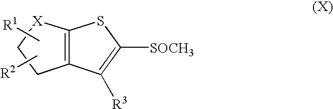

wherein X, R¹, R² and R³ are as defined above.

The reaction is conveniently effected in the presence of a strong base such as sodium hexamethyldisilazide.

The intermediates of formula (X) above may be prepared from the precursors of formula (VII) as defined above by a multi-stage procedure which comprises the following steps: (i) diazotisation/bromination by treatment with tert-butyl nitrite and copper(II) bromide; (ii) treatment of the bromo derivative thereby obtained with dimethyl disulphide, typically in the presence of a strong base such as tert-butyllithium; and subsequently (iii) oxidation of the methylthio derivative thereby obtained, typically with an oxidising agent such as 3-chloroperoxybenzoic acid, to afford the desired compound of formula (X).

Where they are not commercially available, the starting materials of formula (III), (VI), (VIII) and (IX) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound of formula (IA), (IB), (IC) or (ID) wherein Y is oxygen may be converted into the corresponding compound wherein Y is sulphur by treatment with Lawesson's Reagent (i.e. 2,4-bis(4-methoxyphenyl)-1,3-dithia-2,4-diphosphetane-2,4-disulphide). A compound of formula (IB) wherein Y is oxygen may be converted into the corresponding compound of formula (ID) by treatment with hydroxylamine-O-sulfonic acid, typically in the presence of formic acid at an elevated temperature. A compound of formula (IB) wherein Y is oxygen may be converted into the corresponding compound of formula (IE) by treatment with a reducing agent such as lithium aluminium hydride. A compound of formula (IB) wherein Y is oxygen may be converted into the corresponding compound of formula (IF) by treatment with a hydroxylamine derivative of formula H₂N—OR⁷. A compound of formula (IB) wherein Y is oxygen may be converted into the corresponding compound of formula (IG) by treatment with hydrazine hydrate. A compound of formula (IF) may be converted into the corresponding compound of formula (IC) by treatment with p-toluenesulphonyl chloride, typically in the presence of pyridine at an elevated temperature. A compound of formula (IB) wherein Y is oxygen and R¹ is hydrogen may be converted into the corresponding compound wherein R¹ is methyl by treatment with a methyl halide, e.g. iodomethane, in the presence of a strong base, e.g. lithium diisopropylamide. A compound of formula (IC) wherein Y is sulphur may be converted into the corresponding compound wherein Y is NH by treatment with dimethyl sulphate or iodomethane, followed by treatment of the methylthio-substituted cyclic imine thereby obtained with ammonia, typically at elevated temperature and pressure, or with ammonium acetate, typically at elevated temperature.

A compound of formula (IC) wherein R⁶ represents hydrogen may be converted into the corresponding compound wherein R⁶ represents C₁₋₆ alkyl by treatment with a trialkylsilyl halide, e.g. trimethylsilyl chloride or tert-butyldimethylsilyl chloride, in the presence of a base, e.g. sodium hydride, followed by treatment with a C₁₋₆ alkyl halide, e.g. iodomethane, in the presence of a base, e.g. sodium hydride.

A compound of formula (I) wherein R³ represents —CO₂R^{a} in which R¹ is other than hydrogen may be saponified to give the corresponding compound in which R³ represents —CO₂H by treatment with a base such as lithium hydroxide; more prolonged treatment with lithium hydroxide gives rise to the decarboxylated product in which R³ represents hydrogen. A compound of formula (I) wherein R³ represents —CO₂R^{a} may be converted into the corresponding compound wherein R³ represents —CONH₂ by treatment with ammonia, typically at elevated temperature and optionally also at elevated pressure. A compound of formula (I) wherein R³ represents —CO₂H may be converted into the corresponding compound wherein R³ represents —CONR^{b}R^{c}, —CON(OR^{b})R^{c} or —CON(R^{d})C(═NR^{e})NR^{b}R^{c} by treatment with the appropriate amine of formula H—NR^{b}R^{c}, H—N(OR^{b})R^{c} or H—N(R^{d})C(═NR^{e})NR^{b}R^{c} respectively and a condensing agent such as 1,1′-carbonyldiimidazole (CDI) or 1-[3-(dimethylamino)propyl]-3-ethylcarbodiimide (EDC), typically in the presence of 4-methylmorpholine (NMM) and 1-hydroxybenzotriazole (HOBT). Alternatively, a compound of formula (I) wherein R³ represents —CO₂H may be converted into the corresponding compound wherein R³ represents —CONR^{b}R^{c}, —CON(OR^{b})R^{c} or —CON(R^{d})NR^{b}R^{c} by a two-stage procedure which comprises (i) treatment with pentafluorophenol and a condensing agent such as EDC, typically in the presence of NMM and HOBT; and (ii) reaction of the pentafluorophenyl ester thereby obtained with the appropriate amine of formula H—NR^{b}R^{c}, H—N(OR^{b})R^{c} or H—N(R^{d})NR^{b}R^{c} respectively, typically in the presence of an organic base such as triethylamine.

A compound of formula (I) wherein R³ represents —CO₂H may be converted into the corresponding compound wherein R³ represents —CO₂R^{a} by a two-stage procedure which comprises (i) treatment with pentafluorophenol and a condensing agent such as EDC, typically in the presence of NMM and HOBT; and (ii) reaction of the pentafluorophenyl ester thereby obtained with the appropriate alcohol of formula R^{a}OH, typically in the presence of an organic base such as triethylamine.

A compound of formula (I) wherein R³ represents —CO₂H may be converted into the corresponding compound wherein R³ represents —CONR^{b}R^{c} by a two-stage procedure which comprises (i) treatment with tetrafluorophenol resin and a condensing agent such as 1,3-diisopropylcarbodiimide, typically in the presence of 4-(dimethylamino)pyridine; and (ii) reaction of the tetrafluorophenyl ester functionalised resin thereby obtained with the appropriate amine of formula H—NR^{b}R^{c}.

A compound of formula (I) wherein R³ represents —CO₂R^{a} (e.g. ethoxycarbonyl) may be converted into the corresponding compound wherein R³ represents methyl by treatment with a reducing agent such as diisobutylaluminium hydride.

A compound of formula (I) wherein R³ represents —CO₂R^{a} (e.g. ethoxycarbonyl) may be converted into the corresponding compound wherein R³ represents —CONR^{b}R^{c} by treatment with the appropriate amine of formula H—NR^{b}R^{c} in the presence of trimethylaluminium. Similarly, a compound of formula (I) wherein R³ represents cyano may be converted into the corresponding compound wherein R³ represents —C(═NH)NR^{b}R^{c} by treatment with the appropriate amine of formula H—NR^{b}R^{c} in the presence of trimethylaluminium. A compound of formula (I) wherein R³ represents cyano may be converted into the corresponding compound wherein R³ represents 4,4-dimethyl-4,5-dihydro-1H-imidazol-2-yl in a single step by treatment with 1,2-diamino-2-methylpropane in the presence of trimethylaluminium.

A compound of formula (I) wherein R³ represents cyano may be converted into the corresponding compound wherein R³ represents —CONH₂ by treatment with hydroxylamine.

A compound of formula (I) wherein R³ contains a NH functionality may be converted into the corresponding compound wherein R³ contains a N-methyl functionality by treatment with formaldehyde in the presence of a suitable reducing agent, e.g. sodium cyanoborohydride.

A compound of formula (I) wherein R⁵ represents formyl may be converted into the corresponding compound wherein R⁵ represents hydroxymethyl by treatment with a suitable reducing agent, e.g. sodium borohydride.

A compound of formula (I) wherein R⁵ represents iodo may be converted into the corresponding compound wherein R⁵ represents ethynyl by treatment at an elevated temperature with (trimethylsilyl)acetylene and a transition metal catalyst, e.g. bis(triphenylphosphine)palladium(II) dichloride, typically in the presence of copper(I) iodide and a base such as diisopropylamine.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The following Examples illustrate the preparation of compounds according to the invention.

The compounds in accordance with this invention potently inhibit the activity of human MEK enzyme.

### In Vitro MEK Assay

MEK1 activity was measured in a cascade assay initiated by active Raf, via activation of MEK, Erk2 and subsequent phosphorylation of fluorescein-labelled Erk-tide substrate in an assay based on fluorescence polarisation (IMAP). The assay was carried out in 20 mM Tris+5 mM MgCl₂+2 mM DL-dithiothreitol+0.01% Tween 20 pH 7.2, containing 1.5 nM unactive MEK, 100 nM unactive Erk and 200 nM Erk-tide (all concentrations are final concentrations). Compounds, or DMSO controls, were tested at a final concentration of 2% DMSO, and the assay initiated in the presence of 5 μM ATP by addition of 1.25 nM active Raf in assay buffer. After 20 min at r.t., stop solution was added followed by IMAP binding beads, the assay mixture was then incubated for 90 min at r.t. (with shaking) and then read on a Molecular Devices UL HT reader.

When tested in the above assay, the compounds of the accompanying Examples were all found to inhibit human MEK enzyme with IC₅₀ values of 10 μM or better.

### EXAMPLES

Abbreviations used

| | |
|---|---|
| EtOAc—ethyl acetate | DMSO—dimethylsulphoxide |
| THF—tetrahydrofuran | DCM—dichloromethane |
| DMF—N,N-dimethylformamide | NMM—4-methylmorpholine |
| ether—diethyl ether | HOBT—1-hydroxybenzotriazole |
| DAP-OH—2,3-diaminopropionic | CDCl₃—deuterated chloroform |
| acid | |
| BOC—tert-butoxycarbonyl | MeOH—methanol |
| TMS—trimethylsilyl | mCPBA—3-chloroperoxybenzoic |
| | acid |

EDC—1-[3-(dimethylamino)propyl]-3-ethylcarbodiimide

| | |
|---|---|
| h—hour(s) | min—minute(s) |
| r.t.—room temperature | aq—aqueous |
| sat.—saturated | RT—retention time |

All NMRs were obtained either at 300 MHz or 400 MHz.

### Standard LCMS Method

The LC-MS system used comprises a Waters Alliance 2795 HT quaternary HPLC, Waters 996 Photo Diode Array (PDA) detector and Waters ZQ 4000 single quadrupole mass spectrometer. The ZQ can acquire data simultaneously in positive and negative electrospray ionisation modes.

ZQ Mass Spectrometer

| | | | | | |
|---|---|---|---|---|---|
| Capillary | 3.5 | kV | Cone | 50 | V |
| Extractor | 2 | V | Source Temp | 80° | C. |
| Desolvation Temp | 200° | C. | Cone Gas | 150 | l/h |
| Desolvation Gas | 250 | l/h | Multiplier | 650 | V |

Data were acquired in a full scan from 100 to 1000 m/z.

| | |
|---|---|
| Scan duration | 0.80 s |
| Interscan delay | 0.20 s |

### HPLC

The reverse phase separation was carried out on a Gemini C18 from Phenomenex 50×4.6 mm with 5 μm silica.

| | | |
|---|---|---|
| Injection Volume | 5 | μl |
| UV data | 240 to 400 | nm |
| Sample Temperature | 20° | C. |
| Column Temperature | 30° | C. |
| Flow Rate | 0.9 | ml/min |
| Split to ZQ | ~0.40 | ml/min |

Solvent A: 90% 10 mM NH₄CO₂ in water/0.1% formic acid/10% CH₃CN
Solvent B: 90% CH₃CN/0.1% formic acid/10% 10 mM NH₄CO₂ in water

Gradient Program

| Time (min) | A % | B % | Flow | Curve |
|---|---|---|---|---|
| 0.00 | 95.0 | 5.0 | 0.900 | 1 |
| 2.00 | 5.0 | 95.0 | 0.900 | 6 |
| 4.00 | 5.0 | 95.0 | 0.900 | 6 |
| 5.00 | 95.0 | 5.0 | 0.900 | 6 |

The aqueous solvent was approximately pH 3.2.

### Intermediate 1

### 6,6-Dimethylazepane-2,4-dione

Hydroxylamine hydrochloride (39.66 g, 0.57 mol) was added to a solution of dimedone (80 g, 0.57 mol) in MeOH (500 mL) and the reaction was heated to reflux for 4 h. The reaction was concentrated in vacuo to give an orange oil. The resulting oil was dissolved in MeCN (500 mL) and triethylamine (85.6 mL, 0.62 mol) added. The reaction was cooled to 0° C. and a solution of p-toluenesulphonyl chloride (112.0 g, 0.59 mol) in MeCN (600 mL) was added slowly dropwise and the reaction stirred at r.t. for 1 h. Water (32 mL) was added and the reaction heated to 60° C. for 18 h. The reaction was cooled, K₂CO₃ (172 g, 1.24 mol) added, and the suspension was stirred at r.t. for 2 h. The mixture was filtered and the resulting filtrate concentrated in vacuo. The residue was dissolved in DCM (800 mL) and washed with water (100 mL). The combined organic extracts were dried (MgSO₄) and filtered through silica gel (100 g). The resulting filtrate was concentrated in vacuo to give a brown solid. The solid was dissolved in 1:1 THF/heptane (360 mL) and heated to 50° C. for 1.5 h. The mixture was cooled, and the resulting precipitate filtered and dried in vacuo to give the title compound as a beige solid (32.1 g, 36%). δ_{H} (DMSO-d6) 7.89 (1H, br s), 3.43 (2H, s), 3.08 (2H, d, J 6.3 Hz), 2.37 (2H, s), 0.90 (6H, s). LCMS (ES⁺) RT 1.87 minutes, 156 (M+H)⁺.

### Intermediate 2

### 3-Bromo-6,6-dimethylazepane-2,4-dione

N-Bromosuccinimide (11.5 g, 64.5 mmol) was added slowly to a solution of

Intermediate 1 (10.0 g, 64.5 mmol) and NaHSO₄ (1.94 g, 16.1 mmol) in THF (350 mL) at 0° C., and the reaction was warmed to r.t. for 1 h. Aqueous NaHCO₃ (300 mL) was added to the reaction, and the mixture extracted with DCM (3×100 mL). The combined organic extracts were dried (MgSO₄), and concentrated in vacuo to give a white solid. The solid was triturated with isopropyl ether and the resulting precipitate filtered and dried in vacuo to give the title compound as a white solid (11.18 g, 74%). δ_{H} (DMSO-d6) 8.37 (1H, m), 5.74 (1H, s), 3.29 (1H, dd, J 15.1, 6.2 Hz), 2.50-2.70 (2H, m), 2.32 (1H, d, J 11.9 Hz), 0.99 (3H, s), 0.86 (3H, s). LCMS (ES⁺) RT 2.17 minutes, 236 (M+H)⁺.

### Intermediate 3

### 2-Fluoro-4-iodo-1-isothiocyanatobenzene

Thiophosgene (17.8 ml, 232 mmol) was added to a rapidly stirred mixture of 2-fluoro-4-iodoaniline (50.0 g, 211 mmol) in CHCl₃ (500 ml) and water (300 ml). The mixture was stirred at room temperature for 4 hours. The organic phase was separated, washed with saturated sodium bicarbonate solution, dried (Na₂SO₄), filtered and the volatiles removed in vacuo to give the title compound as an off-white crystalline solid (51.7 g, 88%). δ_{H} (DMSO-d₆) 7.87 (1H, dd, J 1.8, 9.5 Hz), 7.63 (1H, ddd, J 1.0, 1.8, 8.4 Hz), 7.25 (1H, dd, J 8.2, 8.4 Hz).

### Intermediate 4

### (2-Fluoro-4-iodophenylthiocarbamoyl)acetic acid ethyl ester

Sodium metal (2 g, 87 mmol) was dissolved in ethanol (250 mL), treated with ethyl acetoacetate (10.72 g, 82.5 mmol) and the reaction stirred for 15 min. Intermediate 3 (23 g, 82.5 mmol) was added portionwise and the reaction stirred at r.t. for 4 h. The reaction mixture was poured onto 2M HCl and extracted into ethyl acetate, dried (Na₂SO₄), filtered and concentrated in vacuo, yielding a red solid. δ_{H} (DMSO-d6) 11.62 (1H, s), 7.78 (1H, dd, J 9.7, 1.8 Hz), 7.63 (1H, m), 7.40 (1H, t, J 8.1 Hz), 4.03 (2H, q, J 7.1 Hz), 3.89 (2H, s), 1.21 (3H, t, J 7.1 Hz). LCMS (ES⁺) RT 2.69 minutes, 366 (M+H)⁺.

### Intermediate 5

### 2-(2-Fluoro-4-iodophenylamino)-5,5-dimethyl-8-oxo-5,6,7,8-tetrahydro-4H-thieno[2,3-c]azepine-3-carboxylic acid pentafluorophenyl ester

1-[3-(Dimethylamino)propyl]-3-ethylcarbodiimide (1.78 g, 9.28 mmol) was added to a mixture of 4-methylmorpholine (1.78 g, 16.88 mmol), 1-hydroxybenzotriazole (1.14 g, 8.44 mmol), pentafluorophenol (1.55 g, 8.44 mmol) and Example 2 (4.0 g, 8.44 mmol) in DCM/DMF (10:1 mixture; 300 mL) and stirred at r.t. for 18 h. Brine (100 mL) was added to the reaction, and the mixture was extracted with DCM (3×100 mL). The combined organic extracts were dried (MgSO₄) and concentrated in vacuo. The residual DMF was azeotroped with heptane. The crude product was triturated with MeCN to give the title compound as a cream solid (3.2 g, 59%). δ_{H} (DMSO, d6) 9.77 (1H, s), 8.05 (1H, t, J 5.0 Hz), 7.84 (1H, dd, J 9.9, 1.8 Hz), 7.69 (1H, dd, J 8.4, 0.9 Hz), 7.44 (1H, t, J 8.4 Hz), 2.92 (2H, s), 2.88 (2H, d, J 5.0 Hz), 0.98 (6H, s). LCMS (ES⁺) RT 3.76 minutes, 641 (M+H)⁺.

### Intermediate 6

### 2-Amino-5,5-dimethyl-8-oxo-5,6,7,8-tetrahydro-4H-thieno[2,3-c]azepine-3-carboxylic acid ethyl ester

Sulphur (2.50 g, 78.1 mmol) and ethyl cyanoacetate (8.3 mL, 78.1 mmol) were added to a solution of Intermediate 1 (10.1 g, 65.1 mmol) in EtOH (30 mL) and the reaction heated to 45° C. for 0.5 h. Morpholine (6.8 mL, 78.1 mmol) was added slowly dropwise and the reaction mixture heated to 65° C. for 18 h. The reaction was cooled to 0° C. and the precipitate filtered and dried in vacuo to give the title compound as a white solid (9.4 g, 51%). δ_{H} (DMSO-d6) 7.73 (1H, t, J 5.1 Hz), 7.69 (2H, br s), 4.21 (2H, q, J 7.1 Hz), 2.82 (2H, s), 2.79 (2H, d, J 5.2 Hz), 1.27 (3H, t, J 7.1 Hz), 0.94 (6H, s). LCMS (ES⁺) RT 2.58 minutes, 283 (M+H)⁺.

### Intermediate 7

### 2-(2-Chloro-4-iodophenylamino)-5,5-dimethyl-8-oxo-5,6,7,8-tetrahydro-4H-thieno[2,3-c]azepine-3-carboxylic acid (2,2-dimethyl-[1,3(S)]dioxolan-4-ylmethyl)amide

1-[3-(Dimethylamino)propyl]-3-ethylcarbodiimide (98 mg, 0.51 mmol) was added to a mixture of 4-methylmorpholine (112 μL, 1.02 mmol), 1-hydroxybenzotriazole (69 mg, 0.51 mmol), (S)-(+)-(2,2-dimethyl-[1,3]dioxolan-4-yl)methylamine (73 mg, 0.56 mmol) and Example 5 (250 mg, 0.51 mmol) in DMF (5 mL) and stirred at r.t. for 18 h. Brine (50 mL) was added to the reaction, and the mixture was extracted with DCM (3×10 mL). The combined organic extracts were dried (MgSO₄) and concentrated in vacuo. The residual DMF was azeotroped with heptane. The crude product was purified by chromatography (silica, 0-30% EtOAc in DCM) to give the title compound as a cream solid (130 mg, 42%). δ_{H} (CDCl₃) 10.51 (1H, s), 7.64 (1H, d, J 2.0 Hz), 7.48 (1H, dd, J 8.6, 2.0 Hz), 7.35 (1H, d, J 8.6 Hz), 6.30 (1H, br s), 6.08 (1H, t, J 5.3 Hz), 4.31-4.23 (1H, m), 4.08-4.03 (1H, m), 3.81-3.73 (1H, m), 3.63 (1H, dd, J 8.5, 6.0 Hz), 3.38-3.29 (1H, m), 2.98 (2H, d, J 4.7 Hz), 2.78 (2H, s), 1.36 (3H, s), 1.24 (3H, s) 1.06 (6H, s). LCMS (ES⁺) RT 3.19 minutes, 604 (M+H)⁺.

### Intermediate 8

### 4-[2-(2-Chloro-4-iodophenylamino)-5,5-dimethyl-8-oxo-5,6,7,8-tetrahydro-4H-thieno[2,3-c]azepin-3-ylcarbonyl]piperazine-1-carboxylic acid tert-butyl ester

Prepared from Example 5 (250 mg, 0.51 mmol) and piperazine-1-carboxylic acid tert-butyl ester (104 mg, 0.56 mmol) by the method of Intermediate 7. The title compound was obtained as a cream solid (100 mg, 30%). δ_{H} (CDCl₃) 7.61 (1H, d, J 2.0 Hz), 7.43 (1H, dd, J 8.6, 2.0 Hz), 7.39 (1H, br s), 7.23 (1H, d, J 8.6 Hz), 6.46 (1H, t, J 4.8 Hz), 3.48-3.33 (6H, m), 3.28-3.17 (2H, m), 2.97 (2H, d, J 4.9 Hz), 2.52 (2H, s), 1.39 (9H, s), 0.98 (6H, s). LCMS (ES⁺) RT 3.15 minutes, 659 (M+H)⁺.

### Intermediate 9

### 2-(2-Chloro-4-iodophenylamino)-5,5-dimethyl-8-oxo-5,6,7,8-tetrahydro-4H-thieno[2,3-c]azepine-3-carboxylic acid (2,2-dimethyl-[1,3(R)]dioxolan-4-ylmethoxy)amide

Prepared from Example 5 (250 mg, 0.51 mmol) and O-(2,2-dimethyl-[1,3]dioxolan-4-ylmethyl)hydroxylamine (prepared according to the procedure described in WO 02/06213; 79 mg, 0.54 mmol) by the method of Intermediate 7. The title compound was obtained as a yellow solid (74 mg, 23%). δ_{H} (MeOH-d4) 7.77 (1H, d, J 2.9 Hz), 7.70-7.53 (4H, m), 7.19 (1H, d, J 8.6 Hz), 4.36-4.30 (1H, m), 4.17-4.05 (2H, m), 3.92-3.75 (2H, m), 3.01 (2H, d, J 4.2 Hz), 2.81 (2H, s), 1.38 (3H, s), 1.33 (3H, s), 1.08 (6H, s). LCMS (ES⁺) RT 3.12 minutes, 620 (M+H)⁺.

### Intermediate 10

### (S)-2-(2,2-Dimethyl-[1,3]dioxolan-4-yl)ethylamine

To a solution of (S)-2,2-dimethyl-1,3-dioxolane-4-acetamide (2.0 g, 12.56 mmol) in THF (60 mL) at 0° C. was added lithium aluminium hydride (1.67 g, 43.95 mmol). The reaction was stirred at room temperature for 2 h. The reaction was quenched by slow addition of 1.7 mL of water, followed by 1.7 mL of aqueous KOH (15% w/v), followed by approximately 3.5 mL of water. The resulting precipitate was removed by filtration, and the filtrate concentrated in vacuo. The title compound was obtained as a yellow oil (1.41 g, 77%) and used crude without further purification.

### Intermediate 11

### 2-(2-Chloro-4-iodophenylamino)-5,5-dimethyl-8-oxo-5,6,7,8-tetrahydro-4H-thieno[2,3-c]azepine-3-carboxylic acid [2-(2,2-dimethyl-[1,3]dioxolan-4-yl)ethyl]amide (S-enantiomer)

Prepared from Example 5 (250 mg, 0.51 mmol) and Intermediate 10 (222 mg, 1.53 mmol) by the method of Intermediate 7. The title compound was obtained as an off-white solid (90 mg, 28%). δ_{H} (CDCl₃) 10.53-10.36 (1H, m), 7.73 (1H, d, J 0.7 Hz), 7.56 (1H, d, J 8.6 Hz), 7.43 (1H, d J 8.6 Hz), 6.44-6.31 (2H, m), 4.33-4.18 (1H, m), 4.17-4.00 (1H, m), 3.87-3.65 (1H, m), 3.65-3.51 (2H, m), 3.07 (2H, s), 2.84 (2H, s), 2.07-1.94 (3H, m), 1.86-1.74 (1H, m), 1.41 (3H, s), 1.35 (3H, s), 1.12 (6H, s). LCMS (ES⁺) RT 3.43 minutes, 618 (M+H)⁺.

### Intermediate 12

### 4-({[2-(2-Chloro-4-iodophenylamino)-5,5-dimethyl-8-oxo-5,6,7,8-tetrahydro-4H-thieno[2,3-c]azepin-3-ylcarbonyl]amino}methyl)piperidine-1-carboxylic acid tert-butyl ester

Prepared from Example 5 (250 mg, 0.51 mmol) and 4-(aminomethyl)piperidine-1-carboxylic acid tert-butyl ester (164 mg, 0.76 mmol) by the method of Intermediate 7. The title compound was obtained as an off-white solid (117 mg, 33%). δ_{H} (CDCl₃) 10.05 (1H, s), 7.62 (1H, d, J 2.0 Hz), 7.44 (1H, dd, J 8.6 2.0 Hz), 7.23 (1H, d, J 8.6 Hz), 6.10-6.00 (2H, br m), 4.10-4.04 (1H, br m), 3.34-3.29 (2H, br m), 2.95 (2H, d, J 5.2 Hz), 2.71 (2H, s), 2.70-2.64 (2H, br m), 1.75-1.56 (4H, br m), 1.38 (9H, s), 1.15-1.06 (2H, br m), 1.02 (6H, s). LCMS (ES⁺) RT 3.82 minutes, 687 (M+H)⁺.

### Intermediate 13

### 2-(2-Chloro-4-iodophenylamino)-5,5,7-trimethyl-8-oxo-5,6,7,8-tetrahydro-4H-thieno[2,3-c]azepine-3-carboxylic acid ethyl ester

To a suspension of Example 4 (478 mg, 1.0 mmol) in THF (5 mL) under nitrogen was added sodium hydride (44 mg, 1.1 mmol). The solution was stirred for 20 minutes before tert-butyldimethylsilyl chloride (109 mg, 1.0 mmol), was added and stirring continued for a further 15 minutes. A second portion of sodium hydride (44 mg, 1.1 mmol) was added and the reaction stirred for 15 minutes. Methyl iodide (142 mg, 1.0 mmol) was added and the reaction stirred at room temperature for 24 hours. The reaction mixture was poured into brine (100 mL) and the mixture extracted with ethyl acetate (3×100 mL). The combined organic extracts were dried (Na₂SO₄), filtered and the solvents removed in vacuo. The crude residue was purified by column chromatography (SiO₂, 0-10% ethyl acetate in dichloromethane) to give the title compound as a pale cream solid (210 mg, 43%). δ_{H} (CDCl₃) 10.87 (1H, s), 7.77 (1H, d, J 2.1 Hz), 7.61 (1H, m), 7.56 (1H, m), 4.42 (2H, q, J 7.1 Hz), 3.19 (3H, s), 3.08 (2H, s), 2.99 (2H, s), 1.44 (3H, t, J 7.1 Hz), 1.09 (6H, s). LCMS (ES⁺) RT 4.60 minutes, 533 (M+H)⁺.

### Intermediate 14

### 2-(2-Chloro-4-iodophenylamino)-5,5,7-trimethyl-8-oxo-5,6,7,8-tetrahydro-4H-thieno[2,3-c]azepine-3-carboxylic acid

Intermediate 13 (130 mg, 0.24 mmol) was dissolved in THF (5 mL) and a solution of lithium hydroxide (21 mg, 0.48 mmol) in water (1 mL) added. The mixture was heated to 75° C. for 18 hours. The volatiles were removed in vacuo and the residue treated with 10% aqueous citric acid solution. The resultant precipitate was filtered and dried under suction to give the title compound as a cream solid (125 mg, quant.). LCMS (ES⁻) RT 2.71 minutes, 503 (M+H)⁻.

### Intermediate 15

### (R)-3-{[2-(2-Fluoro-4-iodophenylamino)-5,5-dimethyl-8-oxo-5,6,7,8-tetrahydro-4H-thieno[2,3-c]azepin-3-ylcarbonyl]amino}piperidine-1-carboxylic acid tert-butyl ester

Prepared from Intermediate 5 (250 mg, 0.39 mmol) and (R)-3-aminopiperidine-1-carboxylic acid tert-butyl ester (78 mg, 0.39 mmol) by the method of Example 19. The title compound was obtained as a yellow solid (130 mg, 51%). δ_{H} (CDCl₃) 10.48 (1H, s), 7.46 (2H, dd, J 10.1, 1.6 Hz), 7.36 (1H, t, J 8.3 Hz), 6.35 (1H, br s), 6.04 (1H, d, J 7.1 Hz), 4.23-4.20 (1H, m), 3.60-3.46 (3H, m), 3.34-3.30 (1H, m), 3.04 (2H, d, J 4.9 Hz), 2.80 (2H, s), 1.92-1.80 (2H, m), 1.67-1.61 (2H, m), 1.45 (9H, s), 1.11 (6H, d, J 3.2 Hz). LCMS (ES⁺) RT 3.75 minutes, 657 (M+H)⁺.

### Intermediate 16

### (S)-3-{[2-(2-Fluoro-4-iodophenylamino)-5,5-dimethyl-8-oxo-5,6,7,8-tetrahydro-4H-thieno[2,3-c]azepin-3-ylcarbonyl]amino}piperidine-1-carboxylic acid tert-butyl ester

Prepared from Intermediate 5 (250 mg, 0.39 mmol) and (S)-3-aminopiperidine-1-carboxylic acid tert-butyl ester (78 mg, 0.39 mmol) by the method of Example 19. The title compound was obtained as a yellow solid (172 mg, 67%). δ_{H} (CDCl₃) 10.51 (1H, s), 7.48-7.44 (2H, m), 7.37 (1H, t, J 8.3 Hz), 6.37 (1H, br s), 5.99 (1H, d, J 7.1 Hz), 4.23-4.17 (1H, m), 3.60-3.52 (3H, m), 3.32-3.26 (1H, m), 3.06 (2H, d, J 4.9 Hz), 2.81 (2H, s), 1.91-1.81 (2H, m), 1.67-1.64 (2H, m), 1.45 (9H, s), 1.13 (6H, d, J 3.6 Hz). LCMS (ES⁺) RT 3.35 minutes, 657 (M+H)⁺.

### Intermediate 17

### (2-{[2-(2-Fluoro-4-iodophenylamino)-5,5-dimethyl-8-oxo-5,6,7,8-tetrahydro-4H-thieno[2,3-c]azepin-3-ylcarbonyl]amino}ethyl)carbamic acid tert-butyl ester

Prepared from Intermediate 5 (250 mg, 0.39 mmol) and tert-butyl-N-(aminoethyl)carbamate (62 mg, 0.39 mmol) by the method of Example 19. The title compound was obtained as a yellow solid (106 mg, 67%). δ_{H} (CDCl₃) 10.57 (1H, s), 7.49-7.45 (2H, m), 7.40-7.35 (1H, m), 6.66 (1H, br s), 6.45 (1H, br s), 4.95 (1H, br s), 3.63-3.57 (2H, m), 3.41-3.48 (2H, m), 3.07 (2H, d, J 4.9 Hz), 2.90 (2H, s), 1.41 (9H, s), 1.13 (6H, s). LCMS (ES⁺) RT 2.85 minutes, 617 (M+H)⁺.

### Intermediate 18

### (4-{[2-(2-Fluoro-4-iodophenylamino)-5,5-dimethyl-8-oxo-5,6,7,8-tetrahydro-4H-thieno[2,3-c]azepin-3-ylcarbonyl]amino}butyl)carbamic acid tert-butyl ester

By the method of Example 32, with (4-aminobutyl)carbamic acid tert-butyl ester (106 mg, 056 mmol). LCMS (ES⁺) RT 3.36 minutes, 645 (M+H)⁺.

### Intermediate 19

### 2-(2-Chloro-4-iodophenylamino)-5,5-dimethyl-8-thioxo-5,6,7,8-tetrahydro-4H-thieno[2,3-c]azepine-3-carboxylic acid ethyl ester

Example 4 (3 g, 2 mmol) and Lawesson's reagent (1.18 g, 2.9 mmol) were dissolved in toluene (90 mL). The reaction was stirred at 110° C. for 2 h. The volatiles were removed in vacuo. The crude product was purified by chromatography (silica, 0-25% ethyl acetate in DCM) to give the title compound as a yellow solid (2.5 g, 85%). δ_{H} (DMSO-d6) 10.27 (1H, s), 8.40-8.30 (1H, m), 7.81 (1H, dd, J 10.3, 1.8 Hz), 7.68 (1H, d, J 8.4 Hz), 7.47-7.40 (1H, m), 4.31 (2H, q, J 7 Hz), 2.93 (2H, d, J 5.4 Hz), 2.84 (2H, s), 1.33 (3H, t, J 7 Hz), 0.99 (6H, s).

### Intermediate 20

### 2-(2-Chloro-4-iodophenylamino)-5,5-dimethyl-8-methylsulfanyl-5,6-dihydro-4H-thieno[2,3-c]azepine-3-carboxylic acid ethyl ester

Intermediate 19 (400 mg, 0.75 mmol), dimethyl sulfate (0.2 mL, 2 mmol) and potassium carbonate (200 mg, 1.44 mmol) were dissolved in DCM (20 mL). The reaction was stirred at room temperature for 48 h. Water (20 mL) was added to the reaction, and the mixture was extracted with DCM (3×30 mL). The combined organic extracts were washed with aqueous saturated sodium bicarbonate solution (150 mL) and brine (150 mL), dried (MgSO₄), filtered and concentrated in vacuo. The crude product was purified by chromatography (silica, 0-20% ethyl acetate in DCM) to give the title compound as an orange solid (130 mg, 31%). δ_{H} (CDCl₃) 10.76 (1H, s), 7.69 (1H, d, J 1.9 Hz), 7.53 (1H, dd, J 8.6, 1.9 Hz), 7.39 (1H, d, J 8.6 Hz), 4.34 (2H, q, J 7.0 Hz), 3.17 (2H, s), 2.67 (2H, s), 2.43 (3H, bs), 1.34 (3H, t, J 7.0 Hz), 1.03 (6H, s). LCMS (ES⁺) RT 2.88 minutes, 548.8 (M+H)⁺.

### Intermediate 21

### 2-(2-Chloro-4-ethynylphenylamino)-5,5-dimethyl-8-oxo-5,6,7,8-tetrahydro-4H-thieno[2,3-c]azepine-3-carboxylic acid ethyl ester

To a stirred solution of Example 4 (500 mg, 0.97 mmol) in diisopropylamine (10 mL) were added (trimethylsilyl)acetylene (95 mg, 0.97 mmol) and Pd(PPh₃)₂Cl₂ (34 mg, 0.05 mmol), followed by CuI (18 mg, 0.10 mmol). The reaction mixture was stirred at 60° C. for 1 h, and then cooled to r.t. Water was added, and the reaction mixture was extracted with DCM (3×100 mL). The combined organic extracts were dried (Na₂SO₄) and concentrated in vacuo. The crude product was purified by chromatography (silica, 0-20% EtOAc in DCM) to give the title compound as a cream solid (263 mg, 65%). δ_{H} (DMSO-d6) 10.81 (1H, s), 8.04 (1H, t, J 4.9 Hz), 7.74-7.71 (2H, m), 7.56 (1H, dd, J 8.5, 1.9 Hz), 4.34 (2H, q, J 7.2 Hz), 4.27 (1H, s), 2.93 (2H, s), 2.86 (2H, d, J 5.1 Hz), 1.34 (3H, t, J 7.2 Hz), 0.99 (6H, s). LCMS (ES⁺) RT 3.77 minutes, 417 (M+H)⁺.

### Intermediate 22

### 2-(2-Chloro-4-ethynylphenylamino)-5,5-dimethyl-8-oxo-5,6,7,8-tetrahydro-4H-thieno[2,3-c]azepine-3-carboxylic acid

Prepared from Intermediate 21 (263 mg, 0.63 mmol) and LiOH by the method of Example 2. The title compound was obtained as a cream solid (199 mg, 65%). δ_{H} (DMSO-d6) 13.08 (1H, br s), 7.87-7.80 (1H, m), 7.67-7.62 (2H, m), 7.51 (1H, dd, J 8.5, 2.1 Hz), 4.19 (1H, s), 3.07 (2H, s), 2.85 (2H, d, J 5.1 Hz), 0.98 (6H, s). One exchangeable proton was not observed. LCMS (ES⁺) RT 3.24 minutes, 389 (M+H)⁺.

### Intermediate 23

### 2-(2-Chloro-4-ethynylphenylamino)-5,5-dimethyl-8-oxo-5,6,7,8-tetrahydro-4H-thieno[2,3-c]azepine-3-carboxylic acid (2,2-dimethyl-[13 (S)]dioxolan-4-ylmethyl)amide

Prepared from Intermediate 22 (199 mg, 0.51 mmol) and (S)-(+)-(2,2-dimethyl-[1,3]dioxolan-4-yl)methylamine (73 mg, 0.56 mmol) by the method of Intermediate 7. The title compound was obtained as a yellow solid (98 mg, 38%). δ_{H} (CDCl₃) 10.68 (1H, s), 7.65 (1H, d, J 8.5 Hz), 7.56 (1H, d, J 1.9 Hz), 7.41 (1H, dd, J 8.5, 1.9 Hz), 6.16 (1H, t, J=5.3 Hz), 6.09 (1H, t, J 5.5 Hz), 4.39-4.32 (1H, m), 4.12 (1H, dd, J 8.5, 6.6 Hz), 3.88 (1H, ddd, J 13.9, 6.6, 3.2 Hz), 3.73 (1H, dd, J 8.5, 6.0 Hz), 3.43 (1H, ddd, J 13.9, 7.3, 4.9 Hz), 3.12-3.05 (3H, m), 2.87 (2H, s), 1.47 (3H, s), 1.38 (3H, s), 1.15 (6H, d, J 2.4 Hz). LCMS (ES⁺) RT 3.21 minutes, 502 (M+H)⁺.

### Intermediate 24

### 2(R)-({[2-(2-Fluoro-4-iodophenylamino)-5,5-dimethyl-8-oxo-5,6,7,8-tetrahydro-4H-thieno[2,3-c]azepin-3-ylcarbonyl]amino}methyl)pyrrolidine-1-carboxylic acid tert-butyl ester

Prepared from Intermediate 5 (250 mg, 0.39 mmol) and (R)-2-(aminomethyl)-pyrrolidine-1-carboxylic acid tert-butyl ester (156 mg, 0.78 mmol) by the method of Example 19. The title compound was obtained as a cream solid (224 mg, 87%). δ_{H} (CDCl₃) 10.78 (1H, br s), 7.79 (1H, br s), 7.47-7.39 (3H, m), 6.39 (1H, br s), 4.10-4.02 (1H, m), 3.73-3.69 (1H, m), 3.48-3.30 (4H, m), 3.16-3.02 (3H, m), 2.14-2.06 (1H, m), 1.99-1.81 (2H, m), 1.79-1.73 (1H, m), 1.44 (9H, s), 1.29 (3H, s), 1.27 (3H, s). LCMS (ES⁺) RT 3.45 minutes, 657 (M+H)⁺.

### Intermediate 25

### 2(S)-({[2-(2-Fluoro-4-iodophenylamino)-5,5-dimethyl-8-oxo-5,6,7,8-tetrahydro-4H-thieno[2,3-c]azepin-3-ylcarbonyl]amino}methyl)pyrrolidine-1-carboxylic acid tert-butyl ester

Prepared from Intermediate 5 (250 mg, 0.39 mmol) and (S)-2-(aminomethyl)-pyrrolidine-1-carboxylic acid tert-butyl ester (156 mg, 0.78 mmol) by the method of Example 19. The title compound was obtained as a cream solid (236 mg, 92%). δ_{H} (CDCl₃) 10.76 (1H, dd, J 1.3, 0.7 Hz), 7.78 (1H, dd, J 1.7, 0.9 Hz), 7.46-7.38 (3H, m), 6.44 (1H, br s), 4.09-4.00 (1H, m), 3.73-3.68 (1H, m), 3.52-3.45 (2H, m), 3.43-3.30 (2H, m), 3.10-3.02 (2H, m), 2.95-2.85 (1H, m), 2.13-2.05 (1H, m), 1.98-1.81 (2H, m), 1.78-1.73 (1H, m), 1.43 (9H, s), 1.15 (3H, s), 1.10 (3H, s). LCMS (ES⁺) RT 3.45 minutes, 657 (M+H)⁺.

### Intermediate 26

### 3(S)-{[2-(2-Fluoro-4-iodophenylamino)-5,5-dimethyl-8-oxo-5,6,7,8-tetrahydro-4H-thieno[2,3-c]azepine-3-carbonyl]amino}pyrrolidine-1-carboxylic acid tert-butyl ester

1,1′-Carbonyldiimidazole (222 mg, 1.37 mmol) and (S)-1-BOC-3-amino-pyrrolidine (234 mg, 1.26 mmol) was added to a solution of Example 2 (500 mg, 1.05 mmol) and the reaction mixture was stirred at RT for 18 h. Aqueous NaOH solution (5%) was added to the reaction and the mixture extracted with DCM (3×100 mL). The combined organic extracts were dried (Na₂SO₄) and concentrated in vacuo. The crude product was purified by chromatography (silica, 0-40% EtOAc in DCM) to give the title compound as a yellow solid (149 mg, 22%). δ_{H} (CDCl₃) 10.06 (1H, s), 7.37-7.32 (2H, m), 7.18-7.12 (1H, m), 6.37-6.31 (2H, m), 4.56 (1H, dd, J 10.1, 4.6 Hz), 3.60 (1H, dd, J 11.5, 5.8 Hz), 3.44-3.37 (2H, m), 3.29-3.20 (1H, m), 2.91 (2H, d, J 5.1 Hz) 2.65 (2H, s), 2.19-2.10 (1H, m), 1.94-1.89 (1H, m), 1.39 (9H, s), 1.00 (6H, d, J 3.4 Hz). LCMS (ES⁺) RT 3.25 minutes, 641 (M+H)⁻.

### Intermediate 27

### {1-[2-(2-Fluoro-4-iodophenylamino)-5,5-dimethyl-8-oxo-5,6,7,8-tetrahydro-4H-thieno[2,3-c]azepin-3-ylcarbonyl]azetidin-3-yl}carbamic acid tert-butyl ester

Prepared from Intermediate 5 (996 mg, 1.56 mmol) and azetidin-3-ylcarbamic acid tert-butyl ester (536 mg, 3.11 mmol) by the method of Example 19. The title compound was obtained as a cream solid (367 mg, 37%). δ_{H} (DMSO-d6) 8.69 (1H, s), 7.92 (1H, t, J 5.0 Hz), 7.69 (1H, dd, J 10.5, 1.8 Hz), 7.56-7.51 (2H, m), 7.10 (1H, t, J 8.7 Hz), 4.12-4.05 (3H, m), 3.84-3.79 (2H, m), 2.91 (2H, d, J 5.0 Hz), 2.63 (2H, s), 1.41 (9H, s), 1.01 (6H, s). LCMS (ES⁺) RT 3.01 minutes, 629 (M+H)⁺.

### Intermediate 28

### 3-{[2-(2-Fluoro-4-iodophenylamino)-5,5-dimethyl-8-oxo-5,6,7,8-tetrahydro-4H-thieno[2,3-c]azepin-3-ylcarbonyl]amino}azetidine-1-carboxylic acid tert-butyl ester

Prepared from Intermediate 5 (350 mg, 0.55 mmol) and 3-aminoazetidine-1-carboxylic acid tert-butyl ester (188 mg, 1.09 mmol) by the method of Example 19. The title compound was obtained as a cream solid (212 mg, 61%). δ_{H} (DMSO-d6) 8.92 (1H, s), 8.52-8.50 (1H, m), 7.92 (1H, br s), 7.62 (1H, dd, J 10.5, 1.9 Hz), 7.46 (1H, d, J 8.4 Hz), 7.06 (1H, t, J 8.8 Hz), 4.48-4.42 (1H, m), 4.04-3.97 (2H, m), 3.63 (2H, dd, J 8.7, 5.5 Hz), 2.87 (2H, d, J 5.1 Hz), 2.68 (2H, s), 1.38 (9H, s), 0.95 (6H, s). LCMS (ES⁺) RT 3.30 minutes, 629 (M+H)⁺.

### Intermediate 29

### 2-(2-Fluoro-4-iodophenylamino)-5,5,7-trimethyl-8-oxo-5,6,7,8-tetrahydro-4H-thieno[2,3-c]azepine-3-carboxylic acid ethyl ester

Example 1 (200 mg, 0.40 mmol) was dissolved in anhydrous THF (6.5 mL) and NaH (60% dispersion in mineral oil, 26 mg, 0.64 mmol) was added. The solution was stirred for 1 h and then cooled to 0° C. TMSCl (43 mg, 0.40 mmol) was added and, after a further 30 min, NaH (60% dispersion in mineral oil, 26 mg, 0.64 mmol) was added. After 90 min, MeI (27 μl, 0.44 mmol) was added and the reaction warmed to room temperature overnight. Brine (20 mL) was added and the aqueous layer was extracted with ether (2×25 mL). The aqueous layer was then neutralised with dilute aqueous HCl and then extracted with DCM (2×25 mL). The combined organic layers were dried (Na₂SO₄) and the solvent removed under reduced pressure. The orange oil was purified by column chromatography on silica gel (DCM→DCM/Et₂O 10%) to yield the title compound (148 mg, 70%). δ_{H} (d₆-DMSO) 10.20 (1H, s), 7.75 (1H, dd, J 10.4, 1.9 Hz), 7.62-7.59 (1H, m), 7.41 (1H, t, J 8.7 Hz), 4.28 (2H, q, J 7.0 Hz), 3.05 (2H, s), 3.00 (3H, s), 2.84 (2H, s), 1.29 (3H, t, J 7.0 Hz), 0.98 (6H, s). LCMS (ES⁺) RT (pH 3) 3.94 minutes, 517 (M+H)⁺.

### Intermediate 30

### 2-(2-Fluoro-4-iodophenylamino)-5,5,7-trimethyl-8-oxo-5,6,7,8-tetrahydro-4H-thieno[2,3-c]azepine-3-carboxylic acid

Intermediate 29 (460 mg, 0.89 mmol) was dissolved in THF/H₂O (3:1, 9 mL) and LiOH (106 mg, 4.43 mmol) was added. The reaction mixture was heated at reflux for 18 h and the THF was then removed under reduced pressure. The remaining aqueous solution was acidified using 10% citric acid and the precipitate was vigorously stirred for 3 h. The solid was filtered, washed with water and dried to yield the title compound as a white solid (354 mg, 81%). δ_{H} (d₆-DMSO) 7.72 (1H, dd, J 10.5, 1.9 Hz), 7.65-7.59 (1H, m), 7.42 (1H, t, J 8.7 Hz), 3.26 (1H, br s), 3.07 (2H, s), 3.03 (3H, s), 2.98 (2H, s), 0.99 (6H, s). One exchangeable proton was not observed. LCMS (ES⁺) RT (pH 10) 1.75 minutes, 489 (M+H)⁺.

### Intermediate 31

### 2-(2-Fluoro-4-iodophenylamino)-5,5,7-trimethyl-8-oxo-5,6,7,8-tetrahydro-4H-thieno[2,3-c]azepine-3-carboxylic acid pentafluorophenyl ester

Intermediate 30 (354 mg, 0.72 mmol) was dissolved in DCM (10 mL) and DMF (1 mL). N-Methylmorpholine (200 mg, 1.97 mmol), HOBT (195 mg, 1.44 mmol), EDC (278 mg, 1.44 mmol) and pentafluorophenol (265 mg, 1.44 mmol) were added sequentially. The reaction mixture was stirred overnight and then brine (30 mL) was added. The aqueous layer was separated and extracted with further DCM (2×30 mL). The combined organics were dried with Na₂SO₄ and the solvent removed under reduced pressure. Any remaining DMF was removed by azeotroping with toluene and the resulting oil was purified using silica gel chromatography (DCM→DCM/EtOAc 10%) to yield the title compound (355 mg, 75%). δ_{H} (d₆-DMSO) 9.78 (1H, s), 7.85 (1H, dd, J 10.0, 1.9 Hz), 7.72-7.67 (1H, m), 7.44 (1H, t, J 8.5 Hz), 3.14 (2H, s), 3.04 (3H, s), 2.89 (2H, s), 1.01 (6H, s). LCMS (ES⁺) RT (pH 3) 4.21 minutes, 655 (M+H)⁺.

### Intermediate 32

### {1-[2-(2-Fluoro-4-iodophenylamino)-5,5,7-trimethyl-8-oxo-5,6,7,8-tetrahydro-4H-thieno[2,3-c]azepin-3-ylcarbonyl]azetidin-3-ylmethyl}carbamic acid tert-butyl ester

Intermediate 31 (300 mg, 0.45 mmol) was dissolved in DCM (4.5 mL) and azetidin-3-ylmethylcarbamic acid tert-butyl ester (167 mg, 0.90 mmol) and triethylamine (125 μl, 0.90 mmol) were added. The reaction mixture was stirred for 3 h and then quenched with brine (20 mL). The aqueous layer was extracted with DCM (3×20 mL) and the combined organics were dried with Na₂SO₄. The solvent was then removed under reduced pressure and the crude material was purified using silica gel chromatography (DCM→DCM/EtOAc 50%) to yield the title compound (240 mg, 81%). δ_{H} (d₆-DMSO) 8.71 (1H, s), 7.63 (1H, dd, J 10.5, 1.9 Hz), 7.42-7.48 (1H, m), 6.99 (1H, t, J 8.5 Hz), 3.90 (2H, m), 3.92-3.84 (2H, m), 3.58 (2H, dd, J 9.4, 5.3 Hz), 3.27 (2H, s), 3.07-3.03 (6H, m), 1.35 (9H, s), 0.98 (6H, s). One exchangeable proton was not observed. LC RT (pH 3) 2.94 minutes.

### Intermediate 33

### 3-Hydroxy-3-(nitromethyl)azetidine-1-carboxylic acid tert-butyl ester

3-Oxoazetidine-1-carboxylic acid tert-butyl ester (500 mg, 2.9 mmol) was dissolved in ethanol (1.5 mL) and to this was added nitromethane (0.6 mL) and triethylamine (catalytic). The reaction mixture was stirred for 18 h and the solvent then removed under reduced pressure to yield the title compound as a white solid (650 mg, 97%). δ_{H} (d₆-DMSO) 6.42 (1H, s), 4.86 (2H, s), 4.04 (2H, d, J 9.2 Hz), 3.75 (2H, d, J 9.2 Hz), 1.39 (9H, s). LCMS (ES⁺) RT (pH 10) 1.73 minutes, 231 (M−H)⁻.

### Intermediate 34

### 3-(Aminomethyl)-3-hydroxyazetidine-1-carboxylic acid tert-butyl ester

Intermediate 33 (500 mg, 2.2 mmol) was dissolved in ethanol (40 mL) in a hydrogenation vessel and 10% palladium on charcoal (43 mg) was added. The vessel was charged with hydrogen to 50 psi and heated to 50° C. This was then stirred for 2 h and the catalyst was removed by filtering through a plug of celite. The solvent was removed under reduced pressure to yield a pale yellow oil. This was purified by chromatography on an amine column using DCM/MeOH 5% as the eluent to afford the title compound (306 mg, 68%). δ_{H} (d₆-DMSO) 5.50 (1H, s), 3.73 (2H, d, J 8.5 Hz), 3.54 (2H, d, J 8.5 Hz), 2.60 (2H, s), 1.37 (9H, s). Some exchangeable protons were not observed.

### Intermediate 35

### Methanesulfonic acid 2-(tert-butoxycarbonylamino)ethyl ester

N—BOC-ethanolamine (9.3 g, 57.7 mmol) and triethylamine (9.7 mL, 69.2 mmol) in DCM were treated with methanesulphonyl chloride (7.93 g, 69.2 mmol) and the reaction mixture stirred at r.t. for 4 h. The reaction mixture was diluted with further DCM and washed with water then dried (sodium sulphate) and concentrated in vacuo to give the title compound as a viscous oil (15 g, quant). δ_{H} (DMSO-d₆) 7.03 (1H, br s), 4.16 (2H, t, J 5.6 Hz), 3.24-3.22 (2H, m), 3.15 (3H, s), 1.38 (9H, s).

### Intermediate 36

### [2-(1,3-Dioxo-1,3-dihydroisoindol-2-yloxy)ethyl]carbamic acid tert-butyl ester

Intermediate 35 (15 g, 62.8 mmol) and N-hydroxyphthalimide (11.4 g, 69 mmol) in DMF (100 mL) with triethylamine (9.7 mL, 69 mmol) were heated at 50° C. for 18 h. The DMF was removed in vacuo and the residue dissolved in toluene, washed with water, concentrated and azeotroped with heptane to remove residual DMF, then concentrated again. The residue was triturated with diethyl ether to yield the title compound. δ_{H} (DMSO-d₆) 7.87 (4H, s), 6.86-6.77 (1H, m), 4.14 (2H, t, J 5.6 Hz), 3.28 (2H, q, J 5.6 Hz), 1.38 (9H, s). LCMS (ES⁺) RT 2.83 minutes, 329 (M+Na)⁺.

### Intermediate 37

### [2-(Aminooxy)ethyl]carbamic acid tert-butyl ester

Intermediate 36 (1 g, 3.3 mmol) in DCM (20 mL) was treated with methyl-hydrazine (158 mg, 3.4 mmol) and the reaction mixture stirred at r.t. for 16 h. The solvent was removed, and the residue was suspended in diethyl ether, filtered and concentrated, to give the title compound. δ_{H} (DMSO-d₆) 6.74-6.65 (1H, br m), 5.99-5.88 (2H, br s), 3.47 (2H, t, J 5.8 Hz), 3.09 (2H, q, J 5.8 Hz), 1.38 (9H, s). LCMS (ES⁻) RT 1.93 minutes, 175 (M−H)⁻.

### Intermediate 38

### tert-Butyl 3-[({2-[(2-fluoro-4-iodophenyl)amino]-5,5,7-trimethyl-8-oxo-5,6,7,8-tetrahydro-4H-thieno[2,3-c]azepin-3-yl}carbonyl)amino]pyrrolidine-1-carboxylate

Intermediate 31(370 mg, 0.57 mmol) and triethylamine (66 mg, 0.57 mmol) in DMF (5 mL) with 1-BOC-3-aminopyrrolidine (207 mg, 1.11 mmol) were stirred at r.t. for 5 days. The solvent was removed in vacuo and the residue azeotroped with heptane then partitioned between ethyl acetate and water. The organic phase was dried (sodium sulphate) and concentrated, and the residue was purified by column chromatography (SiO₂; DCM/ethyl acetate) to yield the title compound. δ_{H} (DMSO-d₆) 8.68 (1H, s), 8.20 (1H, d, J 5.0 Hz), 7.59 (1H, d, J 11.7 Hz), 7.44 (1H, d, J 8.6 Hz), 7.07-6.96 (1H, br m), 4.29-4.18 (1H, br m), 3.42-3.32 (1H, m), 3.29-3.21 (1H, m), 3.08 (2H, s), 3.04 (3H, s), 2.58 (2H, s), 3.04-1.92 (1H, br m), 1.79-1.68 (1H, br m), 1.39 (9H, s), 1.25 (2H, br s), 0.98 (3H, s), 0.96 (3H, s). HPLC RT 3.18 minutes.

### Intermediate 39

### N-(2-Chloro-4-iodophenyl)-2-(cyano)thioacetamide

Acetonitrile (1 mL) was added at −78° C. to a 1M solution of sodium hexamethyldisilazide (3.38 mL, 3.38 mmol) in THF (3 mL) and, after stirring for 15 minutes, (2-chloro-4-iodophenyl)isothiocyanate (500 mg, 1.69 mmol) was added. The reaction was allowed to warm to r.t. and quenched with water, the pH adjusted to pH 7 with 5% citric acid, and the reaction mixture extracted with DCM. The organic phase was separated, dried and concentrated in vacuo and the residue purified by chromatography (SiO₂; 1:1-1:2 hexane:DCM) to give the title compound (350 mg, 61%). δ_{H} (DMSO-d₆) 11.81 (1H, s), 7.97 (1H, d, J 1.9 Hz), 7.76 (1H, dd, J 8.3, 1.9 Hz), 7.20 (1H, d, J 8.3 Hz), 4.27 (2H, s). LCMS (ES⁺) RT 2.82 minutes, 376/378 (M+K)⁺.

### Intermediate 40

### 2-(2-Chloro-4-iodophenylamino)-5,5-dimethyl-8-oxo-5,6,7,8-tetrahydro-4H-thieno[2,3-c]azepine-3-carboxylic acid pentafluorophenyl ester

From Example 5 (6.0 g, 12.2 mmol) by the method of Intermediate 5 to yield the title compound (5.17 g, 65%). δ_{H} (DMSO-d₆) 10.02 (1H, s), 8.08 (1H, t, J 5.0 Hz), 8.03 (1H, d, J 1.9 Hz), 7.84 (1H, dd, J 8.4, 1.9 Hz), 7.53 (1H, d, J 8.4 Hz), 2.94 (2H, s), 2.90 (2H, d, J 5.0 Hz), 0.99 (6H, s). LCMS (ES⁺) RT 4.14 minutes, 657/659 (M+H)⁺.

### Intermediate 41

### 2-(2-Fluoro-4-iodophenylamino)-5,5-dimethyl-8-oxo-5,6,7,8-tetrahydro-4H-thieno[2,3-c]azepine-3-carboxylic acid tetrafluorophenyl resin ester

To a solution of Example 2 (2.56 g, 1.5 eq) in DMF (28 mL) was added tetrafluorophenol resin (2.8 g, 1 eq) followed by the addition of 1,3-diisopropylcarbodiimide (2.54 g, 4.5 eq) and 4-(dimethylamino)pyridine (265 mg, 0.6 eq). The mixture was stirred for 24 hours at room temperature. The desired functionalised resin was then filtered and rinsed twice with sequential washes of DMF, DCM, MeOH then DCM and dried in vacuo.

### Intermediate 42

### 2-(2-Fluoro-4-iodophenylamino)-5,5-dimethyl-8-thioxo-5,6,7,8-tetrahydro-4H-thieno[2,3-c]azepine-3-carboxylic acid ethyl ester

Example 1 (10 g, 20 mmol) and Lawesson's reagent (4.0 g, 10 mmol) were dissolved in toluene (200 mL) and the reaction mixture stirred at 110° C. for 2 h. The reaction was concentrated in vacuo. The crude residue was triturated (1:1) with MeCN and isopropyl ether, and the precipitate filtered off and dried to give the title compound as a yellow solid (7.8 g, 75%). δ_{H} (CDCl₃) 10.72 (1H, d, J 2.0 Hz), 8.06 (1H, br s), 7.46-7.36 (3H, m), 4.33 (2H, q, J 7.1 Hz), 2.97 (2H, d, J 5.8 Hz), 2.91 (2H, s), 1.35 (3H, t, J=7.1 Hz), 1.02 (6H, s). LCMS (ES⁺) RT 3.66 minutes, 519 (M+H)⁺.

### Intermediate 43

### 2-(2-Fluoro-4-iodophenylamino)-5,5-dimethyl-8-methylsulfanyl-5,6-dihydro-4H-thieno[2,3-c]azepine-3-carboxylic acid ethyl ester hydroiodide salt

Intermediate 42 (3.0 g, 5.8 mmol) and MeI (3.6 mL, 58 mmol) were dissolved in MeCN (100 mL) and stirred at r.t. for 18 h. The reaction mixture was concentrated in vacuo. The crude residue was triturated with MeCN and the precipitate was filtered off and dried to give the title compound as a yellow solid (3.3 g, 87%). δ_{H} (DMSO-d6) 11.44 (1H, br s), 10.48 (1H, br s), 7.90 (1H, dd, J 9.9, 1.5 Hz), 7.72 (1H, d, J 8.4 Hz), 7.44 (1H, t, J 8.4 Hz), 4.38 (2H, q, J 7.0 Hz), 3.25 (2H, s), 2.95 (2H, s), 2.79 (3H, s), 1.35 (3H, t, J=7.1 Hz), 1.12 (6H, s). LCMS (ES⁺) RT 2.94 minutes, 533 (M+H)⁺.

### Intermediate 44

### (3,3-Dimethyl-5-oxocyclohexylidene)malononitrile

To a stirred solution of 5,5-dimethylcyclohexane-1,3-dione (98.0 g, 699.1 mmol) and malononitrile (46.2 g, 699.1 mmol) in EtOH (400 mL) at r.t. was added piperidine (10 mL, 99.9 mmol) dropwise over 15 minutes. The reaction mixture was heated to reflux for 3 days, and then concentrated in vacuo. The residue was dissolved in EtOAc (500 mL), and the solution was dried (MgSO₄), filtered and concentrated in vacuo. Purification by column chromatography (SiO₂, 15% MeOH/DCM) gave the title compound (108.2 g, 82%) as a yellow solid. δ_{H} (DMSO-d₆) 8.31 (2H, br s), 2.51 (2H, t, J=1.8 Hz), 2.30 (2H, s), 1.04 (6H, s).

### Intermediate 45

### 2-Amino-5,5-dimethyl-7-oxo-4,5,6,7-tetrahydro-1-benzothiophene-3-carbonitrile

To a stirred solution of Intermediate 44 (50.8 g, 269.9 mmol) and sulphur (10.3 g, 323.9 mmol) in EtOH (600 mL) at r.t. was added morpholine (47.0 mL, 539.8 mmol) dropwise. The reaction mixture was heated to 80° C. for 24 h, and then cooled. The precipitate formed was filtered and washed with cold Et₂O to give the title compound (41.2 g, 53%) as a brown solid that was used without further purification. δ_{H} (DMSO-d₆) 8.31 (2H, br s), 2.51 (2H, t, J 1.8 Hz), 2.30 (2H, s), 1.04 (6H, s). LCMS (ES⁺) 221.0 (M+H)⁺, RT 2.67 minutes.

### Intermediate 46

### 2-Bromo-5,5-dimethyl-7-oxo-4,5,6,7-tetrahydro-1-benzothiophene-3-carbonitrile

To a stirred solution of CuBr₂ (4.2 g, 19.1 mmol) in MeCN (100 mL) at 0° C. was added tert-butyl nitrite (2 mL, 15.0 mmol) dropwise. The reaction mixture was then stirred at this temperature for 10 minutes before Intermediate 45 (3.0 g, 13.6 mmol) was added portionwise. The reaction mixture was then allowed to warm to r.t., stirred for 4 h, and then partitioned between 2M aqueous HCl (200 mL) and EtOAc (3×200 mL). The combined organic fractions were dried (MgSO₄), filtered and concentrated in vacuo. Purification by column chromatography (SiO₂, 25% DCM/hexanes) gave the title compound (1.5 g, 40%) as an off-white solid. δ_{H} (DMSO-d₆) 2.84 (2H, s), 2.51 (2H, s), 1.07 (6H, s).

### Intermediate 47

### 5,5-Dimethyl-2-methylsulfanyl-7-oxo-4,5,6,7-tetrahydrobenzo[b]thiophene-3-carbonitrile

Intermediate 46 (100 mg, 0.35 mmol) and dimethyl disulfide (63 μL, 0.70 mmol) were stirred in THF (3.5 ml) and the mixture cooled to −78° C. tert-Butyllithium (1.7M in pentane, 0.42 ml) was added dropwise and the reaction was stirred for 2 h under an inert atmosphere. A saturated solution of ammonium chloride (10 mL) was added and the reaction mixture was allowed to warm to room temperature. The reaction mixture was extracted with ether (3×15 ml) and the organics combined and dried with sodium sulfate. The solvent was removed under reduced pressure and the crude product was purified using silica gel chromatography (DCM/hexane 1:1→DCM gradient elution) to yield the title compound (49 mg, 44%) contaminated with a 20% impurity. δ_{H} (d₆-DMSO) 2.79 (5H, m), 2.46 (2H, s), 1.06 (6H, s). LCMS (ES⁺) RT (pH 3) 2.73 minutes, 252 (M+H)⁺.

### Intermediate 48

### 2-Methanesulfinyl-5,5-dimethyl-7-oxo-4,5,6,7-tetrahydrobenzo[b]thiophene-3-carbonitrile

Intermediate 47 (100 mg, 0.38 mmol) was dissolved in DCM (4 mL) and the solution cooled to 0° C. mCPBA was added and the reaction was allowed to warm to r.t. After 4 h, the product was washed with a saturated solution of NaHCO₃ (10 mL). The DCM layer was dried with sodium sulfate. The solvent was removed to yield an oil which was triturated with ether/hexane to yield the title compound as a white solid (36 mg, 35%). δ_{H} (d₆-DMSO) 3.58 (3H, s), 2.84 (2H, s), 2.61 (2H, s), 1.09 (6H, s). LC RT (pH 10) 2.24 minutes.

### Intermediate 49

### Ethyl 3-amino-3-methylbutanoate hydrochloride

To a stirred solution of ethyl 3,3-dimethylacrylate (5.0 g, 39.1 mmol) in EtOH (20 mL) in a Parr® reactor at 0° C. was added liquid NH₃ (approximately 20 mL). The reactor was sealed and heated to 90° C. for 24 h. The reaction mixture was then cooled to r.t., bubbled with nitrogen to remove the residual NH₃ and treated with 4M HCl in 1,4-dioxane (10 mL). The reaction mixture was stirred for 30 minutes at r.t. and then evaporated in vacuo to dryness. The resulting grey paste was triturated with DCM, filtered and dried to give the title compound (5.0 g, 70%) as a grey solid that was used without further purification. δ_{H} (CDCl₃) 8.27 (3H, br. s), 4.10 (2H, q, J 7.1 Hz), 2.65 (2H, s), 1.26 (6H, s), 1.20 (3H, t, J 7.1 Hz).

### Intermediate 50

### Ethyl 3-[(3-ethoxy-3-oxopropanoyl)amino]-3-methylbutanoate

To a stirred suspension of Intermediate 49 (5.0 g, 27.4 mmol) in DCM (40 mL) was added triethylamine (11.1 g, 15.3 mL, 109.6 mmol). The reaction mixture was then cooled to 0° C. and ethyl malonyl chloride (4.4 g, 3.7 mL, 28.8 mmol) was added dropwise. The suspension was stirred at r.t. for 2 h before it was diluted with DCM (50 mL) and washed with aqueous 1M HCl (50 mL) and water (2×50 mL). The organics were dried over MgSO₄, filtered and concentrated in vacuo to give the title compound (5.0 g, 71%) as an orange oil that was used without further purification. δ_{H} (DMSO-d₆) 7.75 (1H, br s), 4.15-3.95 (4H, m), 3.14 (2H, s), 2.71 (2H, s), 1.29 (6H, s), 1.21-1.11 (6H, m).

### Intermediate 51

### 6,6-Dimethylpiperidine-2,4-dione

To a stirred solution of NaOEt, prepared in situ from Na (0.53 g, 23.16 mmol) in EtOH (30 mL), was added dropwise a solution of Intermediate 50 (5.00 g, 19.30 mmol) in toluene (30 mL) and the reaction mixture was heated to 80° C. for 2 h. The solution was then concentrated to approximately 10 mL and the residue was dissolved in toluene (30 mL) and extracted with water (3×30 mL). The combined aqueous layers were acidified to pH 2-3 with aqueous 1M HCl and extracted with EtOAc (4×50 mL). The combined organic fractions were dried (MgSO₄), filtered and evaporated in vacuo to give a pale yellow solid that was dissolved in MeCN (90 mL) containing 1% water. The solution was heated to reflux for 2 h and then evaporated in vacuo to dryness. The resulting solid was triturated with isopropyl ether, filtered and dried to give the title compound (1.55 g, 57%) as a cream solid that was used without further purification. Both the keto and enol forms were observed (ratio 3.6:1 keto/enol). δ_{H} (DMSO-d₆) 10.29 (1H, br s, enol), 8.14 (1H, br s, keto), 6.66 (1H, s, enol), 4.81 (1H, s, enol), 3.15 (2H, s), 2.51 (2H, s), 1.20 (6H, s, keto), 1.18 (6H, s, enol).

### Intermediate 52

### Ethyl 2-amino-5,5-dimethyl-7-oxo-4,5,6,7-tetrahydrothieno[2,3-c]pyridine-3-carboxylate

To a stirred solution of sulphur (1.0 g, 31.0 mmol), Intermediate 51 (4.0 g, 28.0 mmol) and ethyl cyanoacetate (3.7 g, 3.5 mL, 29.0 mmol) in EtOH (20 mL) at 45° C. was added morpholine (2.9 g, 2.9 mL, 33.0 mmol) dropwise over 15 minutes. The reaction mixture was stirred at this temperature for 15 minutes and then at 65° C. for 48 h before it was cooled and concentrated in vacuo. To the residue was added water and the resulting solid was filtered and washed with water to give the title compound as a pale brown solid (4.1 g, 54%). δ_{H} (DMSO-d₆) 7.86 (2H, s), 7.28 (1H, s), 4.21 (2H, q, J 7.0 Hz), 2.88 (2H, s), 1.27 (3H, t, J 7.1 Hz), 1.23 (6H, s). LCMS (ES⁺) 269.1 (M+H)⁺.

### Intermediate 53

### Ethyl 2-bromo-5,5-dimethyl-7-oxo-4,5,6,7-tetrahydrothieno[2,3-c]pyridine-3-carboxylate

To a stirred suspension of Intermediate 53 (0.20 g, 0.75 mmol) in MeCN (5 mL) at 0-5° C. was added CuBr₂ (0.20 g, 0.90 mmol) followed by tert-butyl nitrite (0.10 g, 0.10 mL, 0.80 mmol) dropwise. The reaction mixture was stirred at this temperature for 10 minutes before it was partitioned between EtOAc (50 mL) and water (50 mL). The organics were separated, washed with water (3×20 mL) and brine (20 mL), dried (Na₂SO₄), filtered and concentrated in vacuo. The crude residue was washed with Et₂O to give the title compound as a pale brown solid (0.15 g, 61%). δ_{H} (DMSO-d₆) 8.53 (1H, s), 4.32 (2H, q, J 7.0 Hz), 3.10 (2H, s), 1.33 (3H, t, J 7.1 Hz), 1.26 (6H, s). LCMS (ES⁺) 332.0 and 334.0 (M+H)⁺.

### Intermediate 54

### 6-tert-Butyl 3-ethyl 2-bromo-5,5-dimethyl-7-oxo-4,7-dihydrothieno[2,3-c]pyridine-3,6(5H)-dicarboxylate

To a stirred solution of Intermediate 53 (1.0 g, 3.0 mmol) in anhydrous THF (10 mL) cooled to 0° C. was added portionwise sodium hydride (144 mg, 3.6 mmol, 60% dispersion in mineral oil) and the resulting mixture allowed to stir for 30 minutes. Di-tert-butyl dicarbonate (654 mg, 3.0 mmol) was added and the reaction mixture allowed to warm to ambient temperature whereupon anhydrous DMF (5 mL) was added to aid solubility. The resultant solution was stirred for 18 hours. The reaction mixture was poured into saturated brine (100 mL) and extracted with EtOAc (3×100 mL). The combined organic fractions were dried (Na₂SO₄), filtered and the volatiles removed in vacuo. Purification by column chromatography (SiO₂, 90-100% DCM in isohexanes) gave the title compound as a yellow oil (602 mg, 58%). δ_{H} (DMSO-d6) 4.38 (2H, q, J 7.1 Hz), 3.22 (2H, s), 1.55 (9H, s), 1.49 (6H, s), 1.39 (3H, t, J 7.1 Hz). LCMS (ES⁺) RT 3.46 minutes, 434 (M+H)⁺.

### Intermediate 55

### 6-tert-Butyl 3-ethyl 5,5-dimethyl-2-(methylsulfanyl)-7-oxo-4,7-dihydrothieno[2,3-c]pyridine-3,6(5H)-dicarboxylate

To a stirred solution of Intermediate 54 (602 mg, 1.4 mmol) and dimethyl disulphide (0.15 mL, 1.7 mmol) in anhydrous THF (10 mL) cooled to −78° C. was added dropwise tert-butyllithium (1.6 mL, 2.78 mmol, 1.7M in pentane). The resultant reaction mixture was stirred at −78° C. for two hours. The reaction was quenched by addition of 10% aqueous ammonium chloride solution (50 mL) and the aqueous phase extracted with DCM (3×100 mL). The combined organic fractions were dried (Na₂SO₄), filtered and the volatiles removed in vacuo. Purification by column chromatography (SiO₂, 0-60% EtOAc in isohexanes) gave the title compound as a yellow oil (366 mg, 65%). δ_{H} (DMSO-d6) 4.28 (2H, q, J 7.1 Hz), 3.18 (2H, s), 2.65 (3H, s), 1.48 (9H, s), 1.42 (6H, s), 1.29 (3H, t, J 7.1 Hz). LCMS (ES⁺) RT 3.36 minutes, 400 (M+H)⁺.

### Intermediate 56

### 6-tert-Butyl 3-ethyl 5,5-dimethyl-2-(methylsulfinyl)-7-oxo-4,7-dihydrothieno[2,3-c]pyridine-3,6(5H)-dicarboxylate

To a stirred solution of Intermediate 55 (366 mg, 0.92 mmol) in DCM (5 mL) was added 3-chloroperoxybenzoic acid (206 mg, 1.2 mmol). The resultant reaction mixture was stirred at ambient temperature for 18 hours. The reaction was quenched by addition of saturated aqueous NaHCO₃ solution (50 mL) and the aqueous phase extracted with DCM (3×100 mL). The combined organic fractions were dried (Na₂SO₄), filtered and the volatiles removed in vacuo to give a 1:1 mixture of the title compound and the sulphone as a clear oil (258 mg). The material was used in the next step without further purification. LCMS (ES⁺) sulphoxide RT 2.70 minutes, 416 (M+H)⁺; sulphone RT 2.94 minutes, 432 (M+H)⁺.

### Example 1

### 2-(2-Fluoro-4-iodophenylamino)-5,5-dimethyl-8-oxo-5,6,7,8-tetrahydro-4H-thieno[2,3-c]azepine-3-carboxylic acid ethyl ester

Intermediate 2 (11.2 g, 47.6 mmol) was added to a mixture of Intermediate 4 (17.5 g, 47.6 mmol) and caesium carbonate (15.5 g, 47.6 mmol) in DMF (400 mL) and the mixture heated at 80° C. for 18 h. The reaction was cooled and poured onto iced water (200 mL) and 2M HCl added (30 mL). The precipitate was filtered and washed with MeCN to give the title compound as a yellow solid (11.53 g, 48%). δ_{H} (DMSO-d6) 10.32 (1H, s), 7.98 (1H, t, J 4.8 Hz), 7.76 (1H, dd, J 10.4, 1.6 Hz), 7.63 (1H, d, J 8.6 Hz), 7.43 (1H, t, J 8.6 Hz), 4.30 (2H, q, J 7.1 Hz), 2.89 (2H, s), 2.84 (2H, d, J 5.0 Hz), 1.32 (3H, t, J=7.1 Hz), 0.98 (6H, s). LCMS (ES⁺) RT 3.64 minutes, 503 (M+H)⁺.

### Example 2

### 2-(2-Fluoro-4-iodophenylamino)-5,5-dimethyl-8-oxo-5,6,7,8-tetrahydro-4H-thieno[2,3-c]azepine-3-carboxylic acid

Lithium hydroxide monohydrate (1.43 g, 59.8 mmol) was added to a solution of Example 1 (10 g, 19.92 mmol) in THF (20 mL) and water (5 mL) and heated to reflux for 18 h. The reaction mixture was concentrated in vacuo and 5% citric acid (10 mL) added. The resulting precipitate was filtered and dried in vacuo to give the title compound as a yellow solid (7.3 g, 77%). δ_{H} (DMSO, d6) 11.65 (1H, br s), 7.87 (1H, t, J 4.8 Hz), 7.72 (1H, dd, J 10.6, 1.8 Hz), 7.61 (1H, d, J 8.5 Hz), 7.43 (1H, t, J 8.7 Hz), 3.00 (2H, s), 2.84 (2H, d, J 4.9 Hz), 0.97 (6H, s). LCMS (ES⁺) RT 2.98 minutes, 473 (M+H)⁺.

### Example 3

### 2-(2-Fluoro-4-nitrophenylamino)-5,5-dimethyl-8-oxo-5,6,7,8-tetrahydro-4H-thieno[2,3-c]azepine-3-carboxylic acid ethyl ester

Caesium carbonate (142 mg, 0.74 mmol) and 3,4-difluoronitrobenzene (118 mg, 0.74 mmol) were added to a solution of Intermediate 6 (100 mg, 0.34 mmol) in DMF (5 mL) and stirred at r.t. for 18 h. Brine (10 mL) was added to the reaction, and the mixture extracted with EtOAc (3×10 mL). The combined organic extracts were dried (MgSO₄) and concentrated in vacuo. The crude product was triturated with MeOH (5 mL) to give the title compound as a yellow solid (45 mg, 31%). δ_{H} (DMSO-d6) 10.86 (1H, s), 8.25-8.15 (3H, m), 7.72 (1H, t, J 8.5 Hz), 4.30 (2H, q, J 7.0 Hz), 2.91 (2H, s), 2.87 (2H, d, J 4.9 Hz), 1.29 (3H, t, J 7.0 Hz), 0.99 (6H, s). LCMS (ES⁺) RT 3.05 minutes, 422 (M+H)⁺.

### Example 4

### 2-(2-Chloro-4-iodophenylamino)-5,5-dimethyl-8-oxo-5,6,7,8-tetrahydro-4H-thieno[2,3-c]azepine-3-carboxylic acid ethyl ester

Caesium carbonate (2.77 g, 8.51 mmol) and 2-chloro-4-iodo-1-fluorobenzene (2.18 g, 8.51 mmol) were added to a solution of Intermediate 6 (2.0 g, 7.09 mmol) in DMF (20 mL) and heated at 65° C. for 18 h. Brine (100 mL) was added to the reaction and the mixture extracted with DCM (3×50 mL). The combined organic extracts were dried (MgSO₄) and concentrated in vacuo. The residual DMF was azeotroped with heptane. The crude product was purified by chromatography (silica, 0-30% EtOAc in DCM) to give the title compound as a cream solid (916 mg, 25%). δ_{H} (DMSO-d6) 10.60 (1H, s), 8.01 (1H, t, J 5.0 Hz), 7.95 (1H, d, J 2.0 Hz), 7.78 (1H, dd, J 8.6, 2.0 Hz), 7.54 (1H, d, J 8.6 Hz), 4.32 (2H, q, J 7.1 Hz), 2.91 (2H, s), 2.85 (2H, d, J 5.2 Hz), 1.33 (3H, t, J 7.1 Hz), 0.99 (6H, s). LCMS (ES⁺) RT 3.81 minutes, 519 (M+H)⁺.

### Example 5

### 2-(2-Chloro-4-iodophenylamino)-5,5-dimethyl-8-oxo-5,6,7,8-tetrahydro-4H-thieno[2,3-c]azepine-3-carboxylic acid

Lithium hydroxide monohydrate (49 mg, 1.16 mmol) was added to a solution of Example 4 (300 mg, 0.58 mmol) in THF (20 mL) and water (5 mL) and heated to reflux for 18 h. The reaction mixture was concentrated in vacuo and 5% citric acid (10 mL) added. The resulting precipitate was filtered and dried in vacuo to give the title compound as a cream solid (225 mg, 79%). δ_{H} (DMSO-d6) 13.90 (1H, br s), 7.79 (1H, d, J 2.0 Hz), 7.75-7.70 (1H, m), 7.67 (1H, dd, J 8.6, 2.0 Hz), 7.43 (1H, d, J 8.6 Hz), 3.13 (2H, s), 2.82 (2H, d, J 5.0 Hz), 0.96 (6H, s). LCMS (ES⁺) RT 3.10 minutes, 491 (M+H)⁺.

### Example 6

### 2-(2-Chloro-4-iodophenylamino)-5,5-dimethyl-8-oxo-5,6,7,8-tetrahydro-4H-thieno[2,3-c]azepine-3-carboxylic acid (2(S) 3-dihydroxypropyl)amide

A mixture of Intermediate 7 (130 mg, 0.21 mmol) in MeOH (20 mL) and 2M HCl (2 mL) was stirred at r.t. for 1 h. The reaction was concentrated in vacuo to give the title compound as a cream solid (90 mg, 75%). δ_{H} (DMSO-d6) 9.42 (1H, s), 7.96 (1H, t, J 4.6 Hz), 7.82-7.77 (2H, m), 7.64 (1H, dd, J 8.6, 1.9 Hz), 7.22 (1H, d, J 8.6 Hz), 4.76 (1H, d, J 4.9 Hz), 4.54 (1H, t, J 5.5 Hz), 3.58-3.54 (1H, m), 3.39-3.29 (3H, m), 3.28-3.10 (1H, m), 2.88 (2H, d, J 4.9 Hz), 2.77 (2H, s), 0.97 (6H, s). LCMS (ES⁺) RT 2.64 minutes, 562 (M+H)⁺.

### Example 7

### 2-(2-Chloro-4-iodophenylamino)-5,5-dimethyl-3-(piperazin-1-ylcarbonyl)-4,5,6,7-tetrahydrothieno[2,3-c]azepin-8-one hydrochloride

A mixture of Intermediate 8 (100 mg, 0.15 mmol) and 4M HCl in 1,4-dioxane (5 mL) was stirred at ambient temperature for 1 hour. The resulting precipitate was filtered and dried in vacuo to give the title compound as a cream solid (80 mg, 95%). δ_{H} (DMSO-d6) 9.35 (1H, br s), 9.26 (1H, br s), 8.48 (1H, s), 7.90 (1H, t, J 4.9 Hz), 7.82 (1H, d, J 1.9 Hz), 7.59 (1H, dd, J 8.5, 1.9 Hz), 6.97 (1H, d, J 8.5 Hz), 3.61-3.57 (6H, m), 3.12-3.04 (4H, m), 2.87 (2H, d, J 4.8 Hz), 0.96 (6H, s). LCMS (ES⁺) RT 2.19 minutes, 559 (M+H)⁺.

### Example 8

### 2-(2-Chloro-4-iodophenylamino)-5,5-dimethyl-8-oxo-5,6,7,8-tetrahydro-4H-thieno[2,3-c]azepine-3-carboxylic acid methylamide

Prepared from Example 5 (250 mg, 0.51 mmol) and methylamine hydrochloride (36 mg, 0.54 mmol) by the method of Intermediate 7. The title compound was obtained as a cream solid (83 mg, 32%). δ_{H} (DMSO-d6) 9.45 (1H, s), 7.95-7.93 (1H, br m), 7.84-7.81 (2H, br m), 7.64 (1H, dd, J 8.6, 2.0 Hz), 7.22 (1H, d, J 8.6 Hz), 2.90 (2H, d, J 5.0 Hz), 2.75 (3H, s), 2.72 (2H, d, J 4.6 Hz), 0.97 (6H, s). LCMS (ES⁺) RT 3.02 minutes, 504 (M+H)⁺.

### Example 9

### 2-(2-Chloro-4-iodophenylamino)-5,5-dimethyl-3-(pyrrolidin-1-ylcarbonyl)-4,5,6,7-tetrahydrothieno[2,3-c]azepin-8-one

Prepared from Example 5 (250 mg, 0.51 mmol) and pyrrolidine (38 mg, 0.54 mmol) by the method of Intermediate 7. The title compound was obtained as a pink solid (91 mg, 33%). δ_{H} (DMSO-d6) 8.37 (1H, s), 7.92-7.88 (1H, br m), 7.75 (1H, d, J 2.0 Hz), 7.53 (1H, dd, J 8.5, 2.0 Hz), 6.87 (1H, d, J 8.5 Hz), 3.40-3.10 (6H, br m), 2.87 (2H, d, J 4.7 Hz), 1.80-1.65 (4H, br m), 0.94 (6H, s). LCMS (ES⁺) RT 3.02 minutes, 544 (M+H)⁺.

### Example 10

### 2-(2-Chloro-4-iodophenylamino)-5,5-dimethyl-3-(4-methylpiperazin-1-ylcarbonyl)-4,5,6,7-tetrahydrothieno[2,3-c]azepin-8-one

Prepared from Example 5 (250 mg, 0.51 mmol) and 1-methylpiperazine (54 mg, 0.54 mmol) by the method of Intermediate 7. The title compound was obtained as an off-white solid (113 mg, 39%). δ_{H} (CDCl₃) 7.61 (1H, d, J 2.0 Hz), 7.44 (1H, dd, J 8.6, 2.0 Hz), 7.37 (1H, br s), 7.25 (1H, d, J 8.6 Hz), 6.05-5.95 (1H, br m), 3.65-3.40 (4H, br m), 2.96 (2H, d, J 5.3 Hz), 2.53 (2H, s), 2.45-2.44 (2H, br m), 2.42-2.25 (5H, br m), 0.99 (6H, s). LCMS (ES⁺) RT 2.41 minutes, 573 (M+H)⁺.

### Example 11

### 2-(2-Chloro-4-iodophenylamino)-5,5-dimethyl-8-oxo-5,6,7,8-tetrahydro-4H-thieno[2,3-c]azepine-3-carboxylic acid N-methoxy-N-methylamide

Prepared from Example 5 (250 mg, 0.51 mmol) and N,O-dimethylhydroxylamine hydrochloride (53 mg, 0.54 mmol) by the method of Intermediate 7. The title compound was obtained as a pale yellow solid (60 mg, 16%). δ_{H} (DMSO-d6) 8.22 (1H, s), 7.96-7.93 (1H, br m), 7.77 (1H, d, J 2.0 Hz), 7.57 (1H, dd, J 8.6, 2.0 Hz), 6.99 (1H, d J 8.6 Hz), 3.46 (3H, s), 3.16 (3H, s), 2.89 (2H, d, J 5.1 Hz), 2.53 (2H, s), 0.96 (6H, s). LCMS (ES⁺) RT 3.23 minutes, 534 (M+H)⁺.

### Example 12

### 2-(2-Chloro-4-iodophenylamino)-5,5-dimethyl-8-oxo-5,6,7,8-tetrahydro-4H-thieno[2,3-c]azepine-3-carboxylic acid [2-(pyridin-3-yl)ethyl]amide

Prepared from Example 5 (250 mg, 0.51 mmol) and 3-(2-aminoethyl)pyridine (93 mg, 0.76 mmol) by the method of Intermediate 7. The title compound was obtained as a pale green solid (165 mg, 54%). δ_{H} (DMSO-d6) 9.26 (1H, s), 8.44 (1H, d, J 2.8 Hz), 8.39 (1H, dd, J 4.7, 1.6 Hz), 8.00-7.92 (2H, m), 7.65 (1H, d, J 2.0 Hz), 7.64 (2H, dd, J 8.5, 2.0 Hz), 7.27 (1H, dd, J 7.1, 4.7 Hz), 7.20 (1H, d, J 8.5 Hz), 3.54-3.52 (2H, m), 2.86-2.78 (4H, m), 2.56 (2H, s), 0.89 (6H, s). LCMS (ES⁺) RT 3.00 minutes, 595 (M+H)⁺.

### Example 13

### 2-(2-Chloro-4-iodophenylamino)-5,5-dimethyl-8-oxo-5,6,7,8-tetrahydro-4H-thieno[2,3-c]azepine-3-carboxylic acid [2-(pyridin-4-yl)ethyl]amide

Prepared from Example 5 (250 mg, 0.51 mmol) and 4-(2-aminoethyl)pyridine (93 mg, 0.76 mmol) by the method of Intermediate 7. The title compound was obtained as an off-white solid (86 mg, 28%). δ_{H} (DMSO-d6) 9.23 (1H, s), 8.43 (2H, dd, J 4.5, 1.5 Hz), 8.05-7.90 (2H, br m), 7.82 (1H, d, J 2.0 Hz), 7.64 (1H, dd, J 8.6, 2.0 Hz), 7.24 (2H, dd, J 4.5, 1.5 Hz), 7.18 (1H, d, J 8.6 Hz), 3.55-3.49 (2H, m), 2.86-2.78 (4H, m), 2.55 (2H, s), 0.87 (6H, s). LCMS (ES⁺) RT 2.87 minutes, 595 (M+H)⁺.

### Example 14

### 2-(2-Chloro-4-iodophenylamino)-5,5-dimethyl-8-oxo-5,6,7,8-tetrahydro-4H-thieno[2,3-c]azepine-3-carboxylic acid (2-amino-2-methylpropyl)amide

Prepared from Example 5 (250 mg, 0.51 mmol) and 1,2-diamino-2-methylpropane (67 mg, 0.76 mmol) by the method of Intermediate 7. The title compound was obtained as an off-white solid (27 mg, 9%). δ_{H} (DMSO-d6) 8.98 (1H, br s), 8.35 (1H, br s), 7.85 (1H, br m), 7.77 (1H, d, J 2.0 Hz), 7.61 (1H, dd, J 8.6, 2.0 Hz), 7.23 (1H, d, J 8.6 Hz), 3.37-3.33 (4H, br m), 2.91 (2H, s), 2.88 (2H, d, J 4.9 Hz), 1.19 (6H, s), 0.98 (6H, s). LCMS (ES⁺) RT 2.24 minutes, 561 (M+H)⁺.

### Example 15

### 2-(2-Chloro-4-iodophenylamino)-5,5-dimethyl-8-oxo-5,6,7,8-tetrahydro-4H-thieno[2,3-c]azepine-3-carboxylic acid (2(R),3-dihydroxypropoxy)amide

Prepared from Intermediate 9 (74 mg, 0.12 mmol) by the method of Example 6.

The title compound was obtained as an off-white solid (25 mg, 36%). δ_{H} (DMSO-d6) 7.90-7.85 (1H, br m), 7.78 (1H, d, J 1.9 Hz), 7.60 (1H, dd, J 8.6, 1.9 Hz), 7.16 (1H, d, J=8.6 Hz), 3.86-3.81 (1H, m), 3.72-3.67 (2H, m), 3.36 (2H, d, J 4.4 Hz), 2.86 (2H, d, J 5.1 Hz), 2.76-2.72 (2H, br m), 0.97 (6H, s). LCMS (ES⁺) RT 2.66 minutes, 580 (M+H)⁺.

### Example 16

### 2-(2-Chloro-4-iodophenylamino)-5,5-dimethyl-8-oxo-5,6,7,8-tetrahydro-4H-thieno[2,3-c]azepine-3-carboxylic acid (3 (S),4-dihydroxybutyl)amide

Prepared from Intermediate 11(74 mg, 0.12 mmol) by the method of Example 6. The title compound was obtained as an off-white solid (51 mg, 63%). δ_{H} (DMSO-d6) 9.32 (1H, s), 7.97-7.90 (2H, br m), 7.81 (1H, d, J 2.0 Hz), 7.63 (1H, dd, J 8.6, 2.0 Hz), 7.18 (1H, d, J 8.6 Hz), 4.53-4.46 (2H, m), 3.47-3.43 (1H, m), 3.27-3.20 (2H, m), 2.89 (2H, d, J 4.9 Hz), 2.75 (2H, s), 2.54 (2H, s), 1.65-1.61 (1H, br m), 1.43-1.38 (1H, m), 0.97 (6H, s). LCMS (ES⁺) RT 2.51 minutes, 578 (M+H)⁺.

### Example 17

### 2-(2-Chloro-4-iodophenylamino)-5,5-dimethyl-8-oxo-5,6,7,8-tetrahydro-4H-thieno[2,3-c]azepine-3-carboxylic acid (piperidin-4-ylmethyl)amide

Prepared from Intermediate 12 (115 mg, 0.17 mmol) by the method of Example 7. The title compound was obtained as a yellow powder (106 mg, 100%). δ_{H} (DMSO-d6) 9.03 (1H, s), 8.81-8.80 (1H, br m), 8.60-8.50 (1H, br m), 8.18 (1H, t, J 5.9 Hz), 8.07 (1H, t, J 5.0 Hz), 7.84 (1H, d, J 2.0 Hz), 7.66 (1H, dd, J 8.6, 2.0 Hz), 7.11 (1H, d, J 8.6 Hz), 3.29-3.23 (2H, br m), 3.14 (2H, t, J 5.9 Hz), 2.95 (2H, d, J 5.0 Hz), 2.87-2.78 (4H, br m), 1.79-1.74 (3H, br m), 1.43-1.30 (1H, m), 1.03 (6H, s). LCMS (ES⁺) RT 2.20 minutes, 587 (M+H)⁺.

### Example 18

### (S)-2-(2-Chloro-4-iodophenylamino)-5,5,7-trimethyl-8-oxo-5,6,7,8-tetrahydro-4H-thieno[2,3-c]azepine-3-carboxylic acid (2,3-dihydroxypropyl)amide

To a solution of Intermediate 14 (123 mg, 0.24 mmol) in DMF (5 mL) was added 1-[3-(Dimethylamino)propyl]-3-ethylcarbodiimide (47 mg, 0.24 mmol), 1-hydroxy-benzotriazole (33 mg, 0.24 mmol), 4-methylmorpholine (50 mg, 0.48 mmol) and (S)-(+)-(2,2-dimethyl-[1,3]-dioxolan-4-yl)methylamine (36 mg, 0.27 mmol). The mixture was stirred at room temperature for 7 days. 2M HCl (2 mL) was added to the reaction and the mixture stirred for 3 hours. The reaction was poured into brine (50 mL), and extracted with ethyl acetate (3×50 mL). The combined organics were dried (Na₂SO₄), filtered and the solvents removed in vacuo. The crude residue was purified by column chromatography (SiO₂, 20-100% ethyl acetate in dichloromethane) to give the title compound as an off-white solid (62 mg, 44%). δ_{H} (DMSO-d6) 9.26 (1H, s), 7.81-7.80 (2H, m), 7.63 (1H, dd, J2.0, 8.6 Hz), 7.17 (1H, d, J 8.6 Hz), 4.76 (1H, d, J 5.0 Hz), 4.54 (1H, t, J 5.7 Hz), 3.58-3.51 (1H, m), 3.35-3.25 (3H, m), 3.17-3.10 (3H, m), 3.05 (3H, s), 2.70 (2H, s), 0.99 (6H, s). LCMS (ES⁺) RT 2.77 minutes, 578 (M+H)⁺.

### Example 19

### 2-(2-Fluoro-4-iodophenylamino)-5,5-dimethyl-8-oxo-5,6,7,8-tetrahydro-4H-thieno[2,3-c]azepine-3-carboxylic acid (2-hydroxy-2-methylpropyl)amide

1-Amino-2-methylpropan-2-ol (35 mg, 0.39 mmol) was added to a mixture of Intermediate 5 (250 mg, 0.39 mmol) and triethylamine (54 μL, 0.39 mmol) in DCM (5 mL) and stirred at r.t. for 18 h. H₂O (50 mL) was added to the reaction, and the mixture was extracted with DCM (3×10 mL). The combined organic extracts were dried (MgSO₄) and concentrated in vacuo. The crude product was purified by chromatography (silica, 0-40% EtOAc in DCM) to give the title compound as a cream solid (102 mg, 48%). δ_{H} (DMSO-d6) 9.12 (1H, s), 7.94 (1H, br s), 7.72 (1H, br s), 7.62 (1H, dd, J 10.7, 1.8 Hz), 7.49 (1H, d, J 8.6 Hz), 7.12 (1H, t, J 8.7 Hz), 4.49 (1H, s), 3.15 (2H, d, J 5.9 Hz), 2.88 (2H, d, J 5.0 Hz), 2.75 (2H, s), 1.06 (6H, s), 0.96 (6H, s). LCMS (ES⁺) RT 3.24 minutes, 546 (M+H)⁺.

### Example 20

### 2-(2-Fluoro-4-iodophenylamino)-5,5-dimethyl-8-oxo-5,6,7,8-tetrahydro-4H-thieno[2,3-c]azepine-3-carboxylic acid (2-amino-2-methylpropyl)amide

Prepared from Intermediate 5 (250 mg, 0.39 mmol) and 2-methylpropane-1,2-diamine (172 mg, 1.95 mmol) by the method of Example 19. The title compound was obtained as a cream solid (45 mg, 21%). δ_{H} (DMSO-d6) 9.80 (1H, br s), 7.58-7.52 (1H, m), 7.48 (1H, dd, J 10.8, 1.8 Hz), 7.41 (1H, d, J 8.6 Hz), 7.19 (1H, t, J 8.8 Hz), 5.75 (3H, br s), 3.21 (2H, s), 2.96 (2H, s), 2.81 (2H, d, J 4.8 Hz), 1.09 (6H, s), 0.96 (6H, s). LCMS (ES⁺) RT 2.39 minutes, 545 (M+H)⁺.

### Example 21

### 2-(2-Fluoro-4-iodophenylamino)-5,5-dimethyl-8-oxo-5,6,7,8-tetrahydro-4H-thieno[2,3-c]azepine-3-carboxylic acid (1,1-dimethyl-2-hydroxyethyl)amide

Prepared from Intermediate 5 (250 mg, 0.39 mmol) and 2-amino-2-methylpropan-1-ol (35 mg, 0.39 mmol) by the method of Example 19. The title compound was obtained as a cream solid (62 mg, 29%). δ_{H} (DMSO-d6) 8.78 (1H, d, J 1.4 Hz), 7.92 (1H, t, J 5.0 Hz), 7.61 (1H, dd, J 10.7, 1.9 Hz), 7.46 (1H, d, J 8.5 Hz), 7.30 (1H, s), 7.05 (1H, t, J 8.8 Hz), 4.88 (1H, t, J 5.6 Hz), 3.37 (2H, d, J 5.6 Hz), 2.85 (2H, d, J 5.1 Hz), 2.68 (2H, s), 1.15 (6H, s), 0.96 (6H, s). LCMS (ES⁺) RT 3.28 minutes, 546 (M+H)⁺.

### Example 22

### 2-(2-Fluoro-4-iodophenylamino)-5,5-dimethyl-8-oxo-5,6,7,8-tetrahydro-4H-thieno[2,3-c]azepine-3-carboxylic acid (3-aminopropyl)amide

Prepared from Intermediate 5 (250 mg, 0.39 mmol) and propane-1,3-diamine (145 mg, 1.95 mmol) by the method of Example 19. The title compound was obtained as a cream solid (120 mg, 58%). δ_{H} (DMSO-d6) 8.36 (4H, br s), 7.86 (1H, t, J 4.9 Hz), 7.59 (1H, dd, J 10.7, 1.9 Hz), 7.46 (1H, d, J 8.6 Hz), 7.13 (1H, t, J 8.7 Hz), 3.24 (2H, t, J 6.6 Hz), 2.85 (2H, d, J 4.9 Hz), 2.80-2.75 (4H, m), 1.77-1.68 (2H, m), 0.95 (6H, s). LCMS (ES⁺) RT 2.37 minutes, 531 (M+H)⁺.

### Example 23

### (R)-2-(2-Fluoro-4-iodophenylamino)-5,5-dimethyl-8-oxo-5,6,7,8-tetrahydro-4H-thieno[2,3-c]azepine-3-carboxylic acid (piperidin-3-yl)amide hydrochloride

4M HCl in 1,4-dioxane (2 mL) was added to a mixture of Intermediate 15 (130 mg, 0.20 mmol) and 1,4-dioxane (5 mL) in DCM (5 mL) and stirred at r.t. for 1 h. The reaction was concentrated in vacuo to give the title compound as a yellow solid (84 mg, 71%). δ_{H} (DMSO-d6) 9.04 (1H, br s), 8.86 (2H, br s), 8.20 (1H, d, J 7.4 Hz), 7.95 (1H, t, J 4.9 Hz), 7.61 (1H, dd, J 10.7, 1.8 Hz), 7.45 (1H, d, J 8.6 Hz), 7.05 (1H, t, J 8.8 Hz), 4.02 (1H, br s), 3.18-3.10 (2H, m), 2.86 (2H, d, J 4.9 Hz), 2.81-2.68 (4H, m), 1.77-1.62 (3H, m), 1.47-1.23 (1H, m), 0.96 (6H, d, J 1.0 Hz). LCMS (ES⁺) RT 2.39 minutes, 557 (M+H)⁺.

### Example 24

### (S)-2-(2-Fluoro-4-iodophenylamino)-5,5-dimethyl-8-oxo-5,6,7,8-tetrahydro-4H-thieno[2,3-c]azepine-3-carboxylic acid (piperidin-3-yl)amide hydrochloride

Prepared from Intermediate 16 (172 mg, 0.26 mmol) and 4M HCl in 1,4-dioxane (2 mL) by the method of Example 23. The title compound was obtained as a yellow solid (120 mg, 78%). δ_{H} (DMSO-d6) 9.16 (1H, br s), 8.97 (1H, br s), 8.87 (1H, s), 8.24 (1H, d, J 7.8 Hz), 7.95 (1H, t, J 5.0 Hz), 7.61 (1H, dd, J 10.7, 1.9 Hz), 7.45 (1H, d, J 8.5 Hz), 7.05 (1H, t, J 8.7 Hz), 4.04 (1H, br s), 3.18-3.10 (2H, m), 2.86 (2H, d, J 5.0 Hz), 2.81-2.75 (2H, m), 2.69 (2H, d, J 3.6 Hz), 1.84-1.59 (3H, m), 1.48-1.37 (1H, m), 0.96 (6H, d, J 0.9 Hz). LCMS (ES⁺) RT 2.39 minutes, 557 (M+H)⁺.

### Example 25

### 2-(2-Fluoro-4-iodophenylamino)-5,5-dimethyl-8-oxo-5,6,7,8-tetrahydro-4H-thieno[2,3-c]azepine-3-carboxylic acid (3-amino-2-hydroxypropyl)amide

Prepared from Intermediate 5 (250 mg, 0.39 mmol) and 1,3-diamino-2-propanol (450 mg, 1.95 mmol) by the method of Example 19. The title compound was obtained as a yellow solid (45 mg, 21%). δ_{H} (MeOD-d4) 7.56-7.48 (2H, m), 7.22 (1H, t, J 8.7 Hz), 3.78-3.71 (1H, m), 3.59-3.31 (2H, m), 3.03 (2H, s), 2.84 (2H, s), 2.77 (1H, dd, J 13.2, 4.3 Hz), 2.65 (1H, dd, 13.2, 7.3 Hz), 1.08 (6H, s). LCMS (ES⁺) RT 2.36 minutes, 547 (M+H)⁺.

### Example 26

### 2-(2-Fluoro-4-iodophenylamino)-5,5-dimethyl-8-oxo-5,6,7,8-tetrahydro-4H-thieno[2,3-c]azepine-3-carboxylic acid (2-aminoethyl)amide hydrochloride

Prepared from Intermediate 17 (106 mg, 0.17 mmol) and 4M HCl in 1,4-dioxane (2 mL) by the method of Example 23. The title compound was obtained as a yellow solid (52 mg, 55%). δ_{H} (DMSO-d6) 9.35 (1H, s), 8.16 (1H, br s), 8.05 (3H, m), 7.93 (1H, t, J 4.8 Hz), 7.64 (1H, dd, J 10.6, 1.8 Hz), 7.50 (1H, d, J 8.5 Hz), 7.19 (1H, t, J 8.7 Hz), 3.46-3.39 (2H, m), 2.92-2.87 (4H, m), 2.78 (2H, s), 0.97 (6H, s). LCMS (ES⁺) RT 2.06 minutes, 517 (M+H)⁺.

### Example 27

### 2-(2-Fluoro-4-iodophenylamino)-5,5-dimethyl-8-oxo-5,6,7,8-tetrahydro-4H-thieno[2,3-c]azepine-3-carboxylic acid (2-hydroxypropyl)amide

Prepared from Intermediate 5 (250 mg, 0.39 mmol) and 1-amino-2-propanol (59 mg, 0.78 mmol) by the method of Example 19. The title compound was obtained as a cream solid (30 mg, 14%). δ_{H} (DMSO-d6) 9.09 (1H, s), 7.92 (1H, br s), 7.81 (1H, br s), 7.63 (1H, dd, J 10.6, 1.9 Hz), 7.48 (1H, d, J 8.5 Hz), 7.13 (1H, t, J 8.7 Hz), 4.69 (1H, d, J 4.7 Hz), 3.69-3.63 (1H, m), 3.12 (2H, t, J 5.9 Hz), 2.87 (2H, d, J 5.0 Hz), 2.74 (2H, s), 1.02 (3H, d, J 6.2 Hz), 0.96 (6H, s). LCMS (ES⁺) RT 3.12 minutes, 532 (M+H)⁺.

### Example 28

### 2-(2-Fluoro-4-iodophenylamino)-3-(3-hydroxyazetidin-1-ylcarbonyl)-5,5-dimethyl-4,5,6,7-tetrahydrothieno[2,3-c]azepin-8-one

Prepared from Intermediate 5 (250 mg, 0.39 mmol) and 3-hydroxyazetidine hydrochloride (86 mg, 0.78 mmol) by the method of Example 19. The title compound was obtained as a cream solid (62 mg, 30%). δ_{H} (DMSO-d6) 8.69 (1H, s), 7.87 (1H, br s), 7.63 (1H, d, J 10.6 Hz), 7.55 (1H, d, J 8.0 Hz), 7.01 (1H, t, J 8.6 Hz), 5.66 (1H, d, J 5.6 Hz), 4.47-4.39 (1H, m), 4.09-4.03 (2H, m), 3.66-3.64 (2H, m), 2.85 (2H, d, J 4.4 Hz), 2.56 (2H, s), 0.96 (6H, s). LCMS (ES⁺) RT 2.52 minutes, 530 (M+H)⁺.

### Example 29

### 2-(2-Fluoro-4-formylphenylamino)-5,5-dimethyl-8-oxo-5,6,7,8-tetrahydro-4H-thieno[2,3-c]azepine-3-carboxylic acid ethyl ester

Intermediate 6 (0.6 g, 2.13 mmol) in DMF (5 mL) was treated with 3,4-difluoro-benzaldehyde (0.3 g, 2.13 mmol) and Cs₂CO₃ (0.7 g, 2.13 mmol), then the reaction was heated at 80° C. for 18 h. After pouring onto water the solution was extracted into DCM and then toluene. The combined organic phases were dried over Na₂SO₄ and concentrated in vacuo. The crude residue was purified by chromatography (SiO₂, DCM-ethyl acetate) to give the title compound (280 mg). δ_{H} (DMSO-d6) 10.86 (1H, d, J 3.2 Hz), 9.89 (1H, d, J 1.9 Hz), 8.11 (1H, t, J 5.0 Hz), 7.95-7.80 (3H, m), 4.33 (2H, q, J 7.1 Hz), 2.93 (2H, s), 2.87 (2H, d, J 5.1 Hz), 1.32 (3H, t, J 7.1 Hz), 1.00 (6H, s). LCMS (ES⁺) RT 3.04 minutes, 403 (M)⁻.

### Example 30

### 2-[2-Fluoro-4-(hydroxymethyl)phenylamino]-5,5-dimethyl-8-oxo-5,6,7,8-tetrahydro-4H-thieno[2,3-c]azepine-3-carboxylic acid ethyl ester

Example 29 (265 mg, 0.66 mmol) in ethanol (5 mL) was treated with sodium borohydride (25 mg, 0.72 mmol) and stirred at r.t. for 30 minutes. The reaction was quenched with water, the ethanol removed in vacuo and the residue extracted into DCM. After drying over sodium sulphate and evaporation in vacuo the product was chromatographed (SiO₂, DCM-ethyl acetate) to yield the title compound (210 mg). δ_{H} (DMSO-d6) 10.15 (1H, s), 7.94 (1H, t, J 4.8 Hz), 7.58 (1H, t, J 8.4 Hz), 7.29 (1H, d, J 11.9 Hz), 7.24 (1H, d, J 8.5 Hz), 5.31 (1H, t, J 5.8 Hz), 4.51 (2H, d, J 5.7 Hz), 4.32 (2H, q, J 7.1 Hz), 2.91 (2H, s), 2.84 (2H, d, J 5.1 Hz), 1.34 (3H, t, J 7.1 Hz), 1.00 (6H, s). LCMS (ES⁺) RT 2.88 minutes, 407 (M+H)⁺.

### Example 31

### 2-(2-Fluoro-4-iodophenylamino)-5,5-dimethyl-8-oxo-5,6,7,8-tetrahydro-4H-thieno[2,3-c]azepine-3-carboxylic acid hydrazide

Intermediate 5 (200 mg, 0.31 mmol) and 1M hydrazine in THF (0.63 mL, 0.63 mmol) were stirred at r.t. for 18 h. The reaction mixture was partitioned between DCM and 2M NaOH solution, causing a white precipitate to fall out. The precipitate was filtered off, washed with water and dried in vacuo, yielding the title compound. δ_{H} (DMSO-d6) 9.16 (1H, br s), 9.01 (1H, br s), 7.90 (1H, br s), 7.64 (1H, d, J 11.0 Hz), 7.50 (1H, d, J 8.6 Hz), 7.17 (1H, t, J 8.8 Hz), 4.42 (2H, brs), 2.88 (2H, d, J 4.7 Hz), 2.71 (2H, s), 0.96 (6H, s). LCMS (ES⁺) RT 2.62 minutes, 489 (M+H)⁺.

### Example 32

### 2-(2-Fluoro-4-iodophenylamino)-5,5-dimethyl-8-oxo-5,6,7,8-tetrahydro-4H-thieno[2,3-c]azepine-3-carboxylic acid [2-(3H-imidazol-4-yl)ethyl]amide

Example 2 (250 mg, 0.51 mmol), EDC (98 mg, 0.51 mmol), HOBT (69 mg, 0.51 mmol), NMM (103 mg, 1.02 mmol) and 2-(3H-imidazol-4-yl)ethylamine (62 mg, 0.56 mmol) in DMF (5 mL) were stirred at r.t. for 18 h. The reaction was treated with water and DCM, stirred and the organic phase separated then concentrated in vacuo. Residual water and DMF were removed by azeotrope with heptane and the resulting oil chromatographed (SiO₂, DCM-ethyl acetate) to yield the title compound. δ_{H} (DMSO-d6) 9.64 (1H, br s), 8.17 (1H, s), 8.01 (1H, t, J 4.8 Hz), 7.87 (1H, t, J 4.7 Hz), 7.65 (1H, dd, J 1.9, 10.6 Hz), 7.53-7.49 (2H, m), 7.20 (1H, t J 8.7 Hz), 6.81 (1H, s), 3.44-3.38 (2H, m), 2.86 (2H, d, J 4.9 Hz), 2.68-2.63 (4H, m), 0.93 (6H, s). LCMS (ES⁺) RT 1.97 minutes, 568 (M+H)⁺.

### Example 33

### (S)-2-(2-Fluoro-4-iodophenylamino)-5,5-dimethyl-8-oxo-5,6,7,8-tetrahydro-4H-thieno[2,3-c]azepine-3-carboxylic acid (2,3-dihydroxypropyl)amide

Prepared by the method of Example 32, starting from (S)-(2,2-dimethyl-[1,3]dioxolan-4-yl)methylamine (75 mg, 0.56 mmol). δ_{H} (DMSO-d6) 9.31 (1H, s), 8.00 (1H, s), 7.85 (1H, m), 7.73 (1H, d, J 10.7 Hz), 7.59 (1H, d, J 8.6 Hz), 7.26 (1H, t, J 8.8 Hz), 4.86 (1H, d, J 4.9 Hz), 4.64 (1H, t, J 5.8 Hz), 3.68-3.62 (1H, m), 3.46-3.40 (3H, m), 3.26-3.18 (1H, m), 2.96 (2H, d, J 4.9 Hz), 2.83 (2H, s), 1.06 (6H, s). LCMS (ES⁺) RT 2.63 minutes, 548 (M+H)⁺.

### Example 34

### 2-(2-Fluoro-4-iodophenylamino)-5,5-dimethyl-8-oxo-5,6,7,8-tetrahydro-4H-thieno[2,3-c]azepine-3-carboxylic acid (1-methylpiperidin-4-yl)amide

Prepared by the method of Example 32, starting from 1-methylpiperidin-4-ylamine (64 mg, 0.56 mmol). δ_{H} (DMSO-d6) 8.77 (1H, br s), 7.93 (1H, br s), 7.85 (1H, br s), 7.61 (1H, dd, J 10.7, 1.7 Hz), 7.45 (1H, d, J 8.5 Hz), 7.03 (1H, t, J 8.8 Hz), 3.59 (1H, br s), 2.86 (2H, d, J 5.0 Hz), 2.73-2.64 (4H, m), 2.15 (3H, s), 2.01-1.91 (2H, m), 1.65-1.61 (2H, m), 1.48-1.35 (2H, m), 0.96 (6H, s). LCMS (ES⁺) RT 2.27 minutes, 571 (M+H)⁺.

### Example 35

### (S)-2-(2-Fluoro-4-iodophenylamino)-3-(3-hydroxypyrrolidin-1-ylcarbonyl)-5,5-dimethyl-4,5,6,7-tetrahydrothieno[2,3-c]azepin-8-one

Prepared by the method of Example 32, starting from (S)-pyrrolidin-3-ol (49 mg, 0.56 mmol). δ_{H} (DMSO-d6) 8.59-8.43 (1H, m), 7.92-7.81 (1H, m), 7.59 (1H, dd, J 10.6, 1.6 Hz), 7.42 (1H, dd, J 8.5, 1.0 Hz), 6.96 (1H, t, J 8.7 Hz), 4.88 (1H, br s), 4.20 (1H, br s), 3.50-2.99 (5H, m), 2.92-2.79 (2H, m), 2.49-2.34 (1H, m), 1.93-1.62 (2H, m), 0.95 (6H, s). LCMS (ES⁺) RT 2.62 minutes, 544 (M+H)⁺.

### Example 36

### (S)-2-(2-Fluoro-4-iodophenylamino)-3-[2-(hydroxymethyl)pyrrolidin-1-ylcarbonyl]-5,5-dimethyl-4,5,6,7-tetrahydrothieno[2,3-c]azepin-8-one

Prepared by the method of Example 32, starting from (S)-(pyrrolidin-2-yl)-methanol (57 mg, 0.56 mmol). δ_{H} (DMSO-d6) 8.40-7.70 (1H, br s), 7.52 (1H, d, J 9.3 Hz), 7.43 (1H, d, J 8.4 Hz), 7.38 (1H, br s), 7.03 (1H, t, J 8.7 Hz), 4.40-4.10 (1H, br s), 4.01-3.94 (1H, br m), 3.47-3.39 (1H, m), 3.39-3.27 (3H, m), 3.01-2.88 (2H, m), 2.66-2.56 (2H, m), 1.94-1.79 (3H, m), 1.78-1.63 (1H, m), 1.02 (3H, s), 0.99 (3H, s). LCMS (ES⁺) RT 2.82 minutes, 558 (M+H)⁺.

### Example 37

### 2-(2-Fluoro-4-iodophenylamino)-5,5-dimethyl-8-oxo-5,6,7,8-tetrahydro-4H-thieno[2,3-c]azepine-3-carboxylic acid (3-hydroxypropyl)amide

Prepared by the method of Example 32, starting from 3-aminopropan-1-ol (42 mg, 0.56 mmol). δ_{H} (DMSO-d6) 9.10 (1H, br s), 7.90 (2H, br s), 7.60 (1H, d, J 10.6 Hz), 7.47 (1H, d, J 8.6 Hz), 7.12 (1H, t, J 8.8 Hz), 4.45 (1H, t, J 5.0 Hz), 3.42 (2H, q, J 6.0 Hz), 3.22 (2H, q, J 6.7 Hz), 2.87 (2H, d, J 5.0 Hz), 2.72 (2H, s), 1.56 (2H, d, J 6.5 Hz), 0.96 (6H, s). LCMS (ES⁺) RT 2.83 minutes, 532 (M+H)⁺.

### Example 38

### (R)-2-(2-Fluoro-4-iodophenylamino)-5,5-dimethyl-8-oxo-5,6,7,8-tetrahydro-4H-thieno[2,3-c]azepine-3-carboxylic acid (2,3-dihydroxypropoxy)amide

Prepared by the method of Example 32, starting from (R)-3-Aminoxypropane-1,2-diol (83 mg, 0.56 mmol). δ_{H} (DMSO-d6) 11.24 (1H, br s), 8.86 (1H, br s), 7.91 (1H, br s), 7.63 (1H, dd, J 10.6, 1.9 Hz), 7.48 (1H, d, J 8.5 Hz), 7.10 (1H, t, J 8.7 Hz), 4.86 (1H, br s), 4.57 (1H, br s), 3.80 (1H, q, J 7.1 Hz), 3.72-3.61 (2H, m), 3.35 (2H, d, J 4.7 Hz), 2.86 (2H, d, J 5.1 Hz), 2.66 (2H, s), 0.97 (6H, s). LCMS (ES⁺) RT 2.46 minutes, 564 (M+H)⁺.

### Example 39

### 2-(2-Fluoro-4-iodophenylamino)-5,5-dimethyl-8-oxo-5,6,7,8-tetrahydro-4H-thieno[2,3-c]azepine-3-carboxylic acid (4-aminobutyl)amide

Intermediate 18 (200 mg, 0.31 mmol) was dissolved in DCM (5 mL) and treated with 4M HCl in 1,4-dioxane (5 mL), after stirring at r.t. for 2 h. The solvent was removed in vacuo and the residue triturated with methanol, yielding the required product. δ_{H} (DMSO-d6) 9.10 (1H, s), 8.01-7.93 (2H, m), 7.80 (2H, br s), 7.63 (1H, dd, J 10.7, 1.9 Hz), 7.47 (1H, dd, J 8.5, 1.0 Hz), 7.11 (1H, t, J 8.8 Hz), 3.16 (2H, q, J 6.4 Hz), 2.88 (2H, d, J 5.0 Hz), 2.76 (2H, br s), 2.72 (2H, s), 1.60-1.38 (4H, m), 0.97 (6H, s). LCMS (ES⁺) RT 2.36 minutes, 545 (M+H)⁺.

### Example 40

### 2-(2-Chloro-4-iodophenylamino)-8-imino-5,5-dimethyl-5,6,7,8-tetrahydro-4H-thieno[2,3-c]azepine-3-carboxylic acid amide

Intermediate 20 (120 mg, 0.22 mol) was dissolved in 2-(2-ethoxyethoxy)ethanol (7.5 mL) and liquid ammonia (10 mL) added. The reaction was heated in a Parr instrument to 90° C. at 60 atmospheres for 24 h. The residue was dissolved in DCM (100 mL) and washed with water (100 mL). The combined organic extracts were dried (MgSO₄), filtered and concentrated in vacuo. The crude product was purified by chromatography (silica, 9% MeOH, 1% aqueous NH₃ and 89% DCM) to give the title compound as a yellow solid (37 mg, 34%). δ_{H} (DMSO-d6) 9.90 (1H, d, J 4.0 Hz), 8.35-8.32 (1H, m), 7.70 (1H, d, J 2.0 Hz), 7.53 (1H, dd, J 8.5, 2.0 Hz), 7.32 (2H, br s), 7.17 (1H, d, J 8.5 Hz), 6.60 (1H, d, J 4.3 Hz), 3.54-3.34 (4H, m), 2.80 (2H, s), 0.98 (6H, s). LCMS (ES⁺) RT 2.44 minutes, 488.8 (M+H)⁺.

### Example 41

### 2-(2-Chloro-4-iodophenylamino)-8-imino-5,5-dimethyl-5,6,7,8-tetrahydro-4H-thieno[2,3-c]azepine-3-carboxylic acid ethyl ester

Chromatography of the crude residue from the preparation of Example 40 (silica, 9% MeOH, 1% aqueous NH₃ and 89% DCM) gave the title compound as a yellow solid (11 mg, 10%). δ_{H} (DMSO-d6) 10.50 (1H, s), 9.76-9.73 (1H, m), 8.72 (1H, br s), 8.00 (1H, d, J 1.6 Hz), 7.86 (1H, d, J 8.5 Hz), 7.49 (1H, dd, J 8.5, 2.0 Hz), 4.36 (2H, q, J 7.0 Hz), 2.93-2.89 (4H, m), 1.35 (3H, t, J 7.0 Hz), 1.00 (6H, s). LCMS (ES⁺) RT 2.78 minutes, 517.8 (M+H)⁺.

### Example 42

### 2-(2-Fluoro-4-iodophenylamino)-5,5-dimethyl-4,5,617-tetrahydrothieno[2,3-c]azepin-8-one

Example 1(1 g, 2 mmol) and lithium hydroxide (115 mg, 4.8 mmol) were added to a solution of THF/H₂O (150 mL, 2:1) and stirred at 110° C. for 18 h. The crude reaction was acidified (pH=1) using 2N HCl and the mixture stirred at 95° C. until decarboxylation was completed. The reaction was filtered and the filtrate extracted with DCM (3×100 mL). The combined organic extracts were washed with water (300 mL), dried (MgSO₄), filtered and concentrated in vacuo. The crude product was triturated with diethyl ether (7 mL) to give the title compound as a yellow solid (732 mg, 85%). δ_{H} (DMSO-d6) 9.00 (1H, s), 7.68-7.65 (1H, m), 7.59 (1H, dd, J 10.8, 2.0 Hz), 7.46 (1H, d, J 8.5 Hz), 7.27-7.20 (1H, m), 6.36 (1H, s), 2.89 (2H, d, J 5.0 Hz), 2.60 (2H, s), 0.96 (6H, s). LCMS (ES⁺) RT 3.49 minutes, 431 (M+H)⁺.

### Example 43

### 2-(2-Chloro-4-ethynylphenylamino)-5,5-dimethyl-8-oxo-5,6,7,8-tetrahydro-4H-thieno[2,3-c]azepine-3-carboxylic acid (2S,3-dihydroxypropyl)amide

Prepared from Intermediate 23 (98 mg, 0.19 mmol) by the method of Example 6. The title compound was obtained as a yellow solid (10 mg, 11%). δ_{H} (DMSO-d6) 9.50 (1H, s), 7.99 (1H, m), 7.83 (1H, m), 7.59 (1H, d, J 1.5 Hz), 7.40 (2H, m), 4.76 (1H, d, J 4.9 Hz), 4.54 (1H, t, J 5.7 Hz), 4.16 (1H, s), 3.57-3.55 (1H, m), 3.33-3.29 (3H, m), 3.18-3.12 (1H, m), 2.89 (2H, d, J 4.5 Hz), 2.78 (2H, s), 0.98 (6H, s). LCMS (ES⁺) RT 2.45 minutes, 462 (M+H)⁺.

### Example 44

### 2-(2-Fluoro-4-iodophenylamino)-5,5-dimethyl-8-oxo-5,6,7,8-tetrahydro-4H-thieno[2,3-c]azepine-3-carboxylic acid (2-hydroxypropyl)amide

Prepared from Intermediate 5 (250 mg, 0.39 mmol) and 1-aminopropan-2-ol (59 mg, 0.78 mmol) by the method of Example 19. The title compound was obtained as a cream solid (151 mg, 73%). δ_{H} (DMSO-d6) 8.87 (1H, s), 7.92 (1H, t, J 4.9 Hz), 7.67 (1H, s), 7.61 (1H, dd, J 10.7, 1.9 Hz), 7.46 (1H, d, J 8.5 Hz), 7.07 (1H, t, J 8.8 Hz), 4.71 (1H, t, J 5.5 Hz), 3.90-3.86 (1H, m), 3.38-3.21 (2H, m), 2.86 (2H, d, J 5.0 Hz), 2.70 (2H, s), 0.99 (3H, d, J 6.7 Hz), 0.96 (6H, s). LCMS (ES⁺) RT 2.78 minutes, 532 (M+H)⁺.

### Example 45

### 2-(2-Fluoro-4-iodophenylamino)-5,5-dimethyl-3-(morpholin-4-ylcarbonyl)-4,5,6,7-tetrahydrothieno[2,3-c]azepin-8-one

Prepared from Intermediate 5 (250 mg, 0.39 mmol) and morpholine (70 mg, 0.78 mmol) by the method of Example 19. The title compound was obtained as a cream solid (35 mg, 17%). δ_{H} (DMSO-d6) 8.57 (1H, s), 7.89 (1H, br s), 7.61 (1H, d, J 10.4 Hz), 7.41 (1H, d, J 8.0 Hz), 6.95 (1H, t, J 8.5 Hz), 3.46-3.29 (10H, m), 2.87 (2H, s), 0.95 (6H, s). LCMS (ES⁺) RT 2.55 minutes, 542 (M+H)⁻.

### Example 46

### 2-(2-Fluoro-4-iodophenylamino)-5,5-dimethyl-8-oxo-5,6,7,8-tetrahydro-4H-thieno[2,3-c]azepine-3-carboxylic acid N′-(pyridin-2-yl)hydrazide

Prepared from Intermediate 5 (250 mg, 0.39 mmol) and pyridin-2-ylhydrazine (85 mg, 0.78 mmol) by the method of Example 19. The title compound was obtained as a cream solid (24 mg, 11%). δ_{H} (DMSO-d6) 9.85 (2H, br s), 8.78 (1H, s), 7.97 (1H, d, J 4.5 Hz), 7.89-7.84 (1H, m), 7.72 (1H, dd, J 10.4, 1.5 Hz), 7.58-7.50 (2H, m), 7.40 (1H, t, J=8.9 Hz), 6.74-6.66 (2H, m), 2.90 (2H, d, J 4.9 Hz), 2.70 (2H, s), 0.98 (6H, s). LCMS (ES⁺) RT 2.85 minutes, 566 (M+H)⁺.

### Example 47

### 2-(2-Fluoro-4-iodophenylamino)-3-[4-(2-hydroxyethyl)piperazin-1-ylcarbonyl]-5,5-dimethyl-4,5,6,7-tetrahydrothieno[2,3-c]azepin-8-one

Prepared from Intermediate 5 (250 mg, 0.39 mmol) and 2-(piperazin-1-yl)ethanol (102 mg, 0.78 mmol) by the method of Example 19. The title compound was obtained as a cream solid (98 mg, 45%). δ_{H} (DMSO-d6) 8.52 (1H, s), 7.89 (1H, t, J 4.6 Hz), 7.60 (1H, dd, J 10.5, 1.7 Hz), 7.40 (1H, d, J 8.5 Hz), 6.92 (1H, t, J 8.7 Hz), 4.36 (1H, t, J 5.1 Hz), 3.48-3.43 (2H, m), 3.38-3.30 (1H, m), 2.86 (2H, d, J 4.5 Hz), 2.50-2.46 (5H, m), 2.35-2.29 (6H, m), 0.95 (6H, s). LCMS (ES⁺) RT 2.44 minutes, 587 (M+H)⁺.

### Example 48

### 3-{[2-(2-Fluoro-4-iodophenylamino)-5,5-dimethyl-8-oxo-5,6,7,8-tetrahydro-4H-thieno[2,3-c]azepin-3-ylcarbonyl]amino}propionic acid ethyl ester

Prepared from Intermediate 5 (250 mg, 0.39 mmol) and 3-aminopropionic acid ethyl ester (120 mg, 1.02 mmol) by the method of Example 19. The title compound was obtained as a cream solid (82 mg, 37%). δ_{H} (DMSO-d6) 9.08 (1H, s), 7.98-7.91 (2H, m), 7.63 (1H, d, J 10.6 Hz), 7.49 (1H, d, J 8.3 Hz), 7.12 (1H, t, J 8.8 Hz), 4.04 (2H, q, J 7.0 Hz), 3.38 (2H, d, J 5.8 Hz), 2.86 (2H, d, J 4.0 Hz), 2.68 (2H, s), 2.47-2.43 (2H, s), 1.17 (3H, t, J 7.0 Hz), 0.95 (6H, s). LCMS (ES⁺) RT 3.27 minutes, 574 (M+H)⁺.

### Example 49

### {1-[2-(2-Fluoro-4-iodophenylamino)-5,5-dimethyl-8-oxo-5,6,7,8-tetrahydro-4H-thieno[2,3-c]azepin-3-ylcarbonyl]azetidin-3-ylmethyl}carbamic acid tert-butyl ester

Prepared from Intermediate 5 (250 mg, 0.39 mmol) and (azetidin-3-ylmethyl)-carbamic acid tert-butyl ester (145 mg, 0.78 mmol) by the method of Example 19. The title compound was obtained as a cream solid (236 mg, 94%). δ_{H} (DMSO-d6) 8.70 (1H, s), 7.86 (1H, t, J 5.0 Hz), 7.63 (1H, dd, J 10.5, 1.9 Hz), 7.46 (1H, d, J 8.5 Hz), 7.01 (1H, t, J=8.7 Hz), 6.98-6.95 (1H, m), 4.02-3.85 (2H, m), 3.59 (2H, dd, J 9.2, 5.3 Hz), 3.30-3.27 (1H, m), 3.08-3.04 (2H, m), 2.85 (2H, d, J 5.1 Hz), 2.56 (2H, s), 1.35 (9H, s), 0.96 (6H, s). LCMS (ES⁺) RT 3.14 minutes, 643 (M+H)⁺.

### Example 50

### 3(R)-3-{[2-(2-Fluoro-4-iodophenylamino)-5,5-dimethyl-8-oxo-5,6,7,8-tetrahydro-4H-thieno[2,3-c]azepin-3-ylcarbonyl]amino}pyrrolidine-1-carboxylic acid tert-butyl ester

Prepared from Intermediate 5 (250 mg, 0.39 mmol) and (R)-1-BOC-3-amino-pyrrolidine (145 mg, 0.78 mmol) by the method of Example 19. The title compound was obtained as a cream solid (207 mg, 83%). δ_{H} (DMSO-d6) 8.80 (1H, br s), 8.17 (1H, br s), 7.93 (1H, br s), 7.60 (1H, d, J 9.9 Hz), 7.46 (1H, d, J 8.3 Hz), 7.09-7.01 (1H, m), 4.25-4.20 (1H, m), 3.38 (1H, dd, J 10.7, 6.2 Hz), 3.29-3.26 (1H, m), 3.15-3.06 (1H, m), 2.86 (2H, d, J 4.9 Hz), 2.66 (2H, s), 1.99-1.90 (1H, m), 1.81-1.69 (1H, m), 1.39 (10H, s), 0.95 (6H, d, J 3.4 Hz). LCMS (ES⁺) RT 3.38 minutes, 643 (M+H)⁺.

### Example 51

### 3-([1,4]Diazepan-1-ylcarbonyl)-2-(2-fluoro-4-iodophenylamino)-5,5-dimethyl-4,5,6,7-tetrahydrothieno[2,3-c]azepin-8-one

Prepared from Intermediate 5 (250 mg, 0.39 mmol) and [1,4]diazepane (78 mg, 0.78 mmol) by the method of Example 19. The title compound was obtained as a cream solid (118 mg, 54%). δ_{H} (DMSO-d₆; 120° C.) 7.52 (1H, dd, J 10.6, 1.8 Hz), 7.43 (2H, d, J=8.5 Hz), 7.05 (1H, t, J 8.7 Hz), 3.60-3.52 (1H, m), 3.44-3.39 (3H, m), 2.94 (2H, d, J 5.1 Hz) 2.84-2.76 (5H, m), 2.53 (2H, s), 1.68-1.60 (2H, m), 1.00 (6H, s). One exchangeable proton was not observed. LCMS (ES⁺) RT 2.34 minutes, 557 (M+H)⁺.

### Example 52

### 2-(2-Fluoro-4-iodophenylamino)-5,5-dimethyl-8-oxo-5,6,7,8-tetrahydro-4H-thieno[2,3-c]azepine-3-carboxylic acid 2-(dimethylamino)ethylamide

Prepared from Intermediate 5 (250 mg, 0.39 mmol) and N,N-dimethylethylene-diamine (69 mg, 0.78 mmol) by the method of Example 19. The title compound was obtained as a cream solid (115 mg, 54%). δ_{H} (DMSO-d6) 9.22 (1H, br s), 7.88-7.84 (2H, m), 7.66 (1H, dd, J 10.5, 1.3 Hz), 7.50 (1H, d, J 8.3 Hz), 7.14 (1H, t, J 8.7 Hz), 3.29-3.26 (2H, m), 2.88 (2H, d, J 4.7 Hz), 2.71 (2H, s), 2.27 (2H, t, J 6.1 Hz), 2.14 (6H, s), 0.96 (6H, s). LCMS (ES⁺) RT 2.52 minutes, 545 (M+H)⁺.

### Example 53

### 2-(2-Fluoro-4-iodophenylamino)-5,5-dimethyl-8-oxo-5,6,7,8-tetrahydro-4H-thieno[2,3-c]azepine-3-carboxylic acid 2-(morpholin-4-yl)ethylamide

Prepared from Intermediate 5 (250 mg, 0.39 mmol) and 2-(morpholin-4-yl)-ethylamine (102 mg, 0.78 mmol) by the method of Example 19. The title compound was obtained as a cream solid (136 mg, 60%). δ_{H} (DMSO-d6) 9.16 (1H, s), 7.92 (1H, t, J 4.7 Hz), 7.78 (1H, t, J 5.3 Hz), 7.65 (1H, dd, J 10.5, 1.7 Hz), 7.50 (1H, d, J 8.7 Hz), 7.13 (1H, t, J 8.8 Hz), 3.52-3.49 (2H, m), 3.30 (2H, d, J 5.6 Hz), 2.89 (2H, d, J 4.9 Hz), 2.74 (2H, s), 2.49-2.30 (6H, m), 2.07 (2H, s), 0.98 (6H, s). LCMS (ES⁺) RT 2.55 minutes, 587 (M+H)⁺.

### Example 54

### 2-(2-Fluoro-4-iodophenylamino)-5,5-dimethyl-8-oxo-5,6,7,8-tetrahydro-4H-thieno[2,3-c]azepine-3-carboxylic acid 3-(dimethylamino)-2,2-dimethylpropylamide

Prepared from Intermediate 5 (250 mg, 0.39 mmol) and N,N,2,2-tetramethyl-1,3-propanediamine (102 mg, 0.78 mmol) by the method of Example 19. The title compound was obtained as a cream solid (124 mg, 54%). δ_{H} (DMSO-d6) 9.10 (1H, br s), 8.00-7.92 (2H, m), 7.62 (1H, dd, J 10.7, 1.9 Hz), 7.48 (1H, d, J 8.5 Hz), 7.09 (1H, t, J 8.7 Hz), 3.09 (2H, s), 2.89 (2H, d, J 4.9 Hz), 2.76 (2H, s), 2.25-2.05 (8H, m), 0.97 (6H, s), 0.82 (6H, s). LCMS (ES⁺) RT 2.56 minutes, 587 (M+H)⁺.

### Example 55

### 2-(2-Fluoro-4-iodo-phenylamino)-5,5-dimethyl-8-oxo-5,6,7,8-tetrahydro-4H-thieno[2,3-c]azepine-3-carboxylic acid 3-(4-methylpiperazin-1-yl)propylamide

Prepared from Intermediate 5 (250 mg, 0.39 mmol) and 3-(4-methylpiperazin-1-yl)propylamine (123 mg, 0.78 mmol) by the method of Example 19. The title compound was obtained as a cream solid (96 mg, 40%). δ_{H} (DMSO-d6) 8.70 (1H, br s), 7.68 (1H, br s), 7.35 (1H, d, J 10.7 Hz), 7.21 (1H, d, J 8.6 Hz), 6.80 (1H, t, J 8.8 Hz), 3.05 (2H, s), 2.90 (2H, t, J 6.5 Hz), 2.63 (2H, d, J 4.8 Hz), 2.46 (2H, s), 2.14-1.96 (9H, m),1.89 (3H, s), 1.30-0.99 (2H, m), 0.72 (6H, s). LCMS (ES⁺) RT 2.43 minutes, 614 (M+H)⁺.

### Example 56

### 2-(2-Fluoro-4-iodophenylamino)-5,5-dimethyl-8-oxo-5,6,7,8-tetrahydro-4H-thieno[2,3-c]azepine-3-carboxylic acid [(2R)-pyrrolidin-2-ylmethyl]amide

Trifluoroacetic acid (2 mL) was added to a solution of Intermediate 24 (224 mg, 0.34 mmol) in DCM (2 mL) and the reaction mixture was stirred at r.t. for 18 h. Saturated aqueous NaHCO₃ solution was added to the reaction until pH=8 and the aqueous fraction was extracted with DCM (3×100 mL). The combined organic extracts were dried (Na₂SO₄) and concentrated in vacuo. The crude product was purified by chromatography (silica, 10% MeOH in DCM) to give the title compound as a yellow solid (128 mg, 68%). δ_{H} (DMSO-d6) 9.10 (1H, br s), 7.72 (1H, br s), 7.56 (1H, d, J 10.7 Hz), 7.45 (1H, d, J 8.7 Hz), 7.18-7.12 (1H, m), 3.40-3.25 (3H, m), 2.90-2.80 (6H, m), 1.75-1.60 (3H, m), 1.52-1.41 (1H, m), 0.96 (6H, s). Two exchangeable protons were not observed. LCMS (ES⁺) RT 2.55 minutes, 557 (M+H)⁺.

### Example 57

### 2-(2-Fluoro-4-iodophenylamino)-5,5-dimethyl-8-oxo-5,6,7,8-tetrahydro-4H-thieno[2,3-c]azepine-3-carboxylic acid [2(S)-pyrrolidin-2-ylmethyl]amide

Prepared from Intermediate 25 (236 mg, 0.36 mmol) by the method of Example 56 to give the title compound as a yellow solid (117 mg, 56%). δ_{H} (DMSO-d6) 9.19 (1H, br s), 7.70 (1H, br s), 7.56 (1H, d, J 10.7 Hz), 7.45 (1H, d, J 8.7 Hz), 7.16 (1H, t, J 8.7 Hz), 3.35-3.26 (3H, m), 2.89-2.78 (6H, m), 1.76-1.68 (3H, m), 1.43-1.38 (1H, m), 0.96 (6H, s). Two exchangeable protons were not observed. LCMS (ES⁺) RT 2.54 minutes, 557 (M+H)⁺.

### Example 58

### 2-(2-Fluoro-4-iodophenylamino)-5,5-dimethyl-3-(4-methylpiperazin-1-ylcarbonyl)-4,5,6,7-tetrahydrothieno[2,3-c]azepin-8-one

Prepared from Intermediate 5 (250 mg, 0.39 mmol) and 1-methylpiperazine (90 mg, 0.90 mmol) by the method of Example 19. The title compound was obtained as a cream solid (32 mg, 15%). δ_{H} (DMSO-d6) 8.51 (1H, s), 7.89 (1H, br s), 7.60 (1H, d, J 10.6 Hz), 7.40 (1H, d, J 8.3 Hz), 6.91 (1H, t, J 8.6 Hz), 2.87-2.83 (2H, m), 2.58-2.45 (6H, m), 2.17-2.12 (7H, m), 0.94 (6H, s). LCMS (ES⁺) RT 2.36 minutes, 557 (M+H)⁺.

### Example 59

### 2-(2-Fluoro-4-iodophenylamino)-5,5-dimethyl-8-oxo-5,6,7,8-tetrahydro-4H-thieno[2,3-c]azepine-3-carboxylic acid [3(S)-pyrrolidin-3-yl]amide hydrochloride

Prepared from Intermediate 26 (149 mg, 0.23 mmol) by the method of Example 7 to give the title compound as a yellow solid (82 mg, 61%). δ_{H} (DMSO-d6) 9.39 (1H, br s), 9.23 (1H, br s), 8.98 (1H, s), 8.39 (1H, d, J 6.3 Hz), 7.93 (1H, br s), 7.62 (1H, d, J 10.2 Hz), 7.48 (1H, d, J 7.8 Hz), 7.09 (1H, t, J 8.5 Hz), 4.49-4.30 (1H, m), 3.39-3.14 (3H, m), 3.55-3.42 (1H, m), 3.09-3.02 (1H, m), 2.88 (2H, d, J 3.4 Hz), 2.73 (2H, s), 2.11-2.04 (1H, m), 1.81-1.72 (1H, m), 0.97 (6H, s). LCMS (ES⁺) RT 2.71 minutes, 542 (M+H)⁺.

### Example 60

### 2-(2-Fluoro-4-iodophenylamino)-5,5-dimethyl-8-oxo-5,6,7,8-tetrahydro-4H-thieno[2,3-c]azepine-3-carboxylic acid [3(R)-pyrrolidin-3-yl]amide hydrochloride

Prepared from Example 50 (192 mg, 0.30 mmol) by the method of Example 7 to give the title compound as a yellow solid (54 mg, 33%). δ_{H} (DMSO-d6) 9.30 (1H, br s), 9.16 (1H, br s), 8.89 (1H, s), 8.31 (1H, d, J 6.1 Hz), 7.85 (1H, br s), 7.54 (1H, d, J 10.6 Hz), 7.39 (1H, d, J 7.8 Hz), 7.00 (1H, t, J 8.6 Hz), 4.40-4.30 (1H, m), 4.11-3.95 (1H, m), 3.39-3.14 (4H, m), 2.96-2.73 (2H, m), 2.64 (2H, s), 2.00-1.92 (1H, m), 1.73-1.65 (1H, m), 0.88 (6H, s). LCMS (ES⁺) RT 2.72 minutes, 543 (M+H)⁺.

### Example 61

### 3-({[2-(2-Fluoro-4-iodophenylamino)-5,5-dimethyl-8-oxo-5,6,7,8-tetrahydro-4H-thieno[2,3-c]azepin-3-ylcarbonyl]amino}methyl)azetidine-1-carboxylic acid tert-butyl ester

Prepared from Intermediate 5 (250 mg, 0.39 mmol) and 3-(aminomethyl)-azetidine-1-carboxylic acid tert-butyl ester (145 mg, 0.78 mmol) by the method of Example 19. The title compound was obtained as a cream solid (211 mg, 84%). δ_{H} (DMSO-d6) 8.99 (1H, s), 8.11 (1H, t, J 5.3 Hz), 7.94 (1H, t, J 4.7 Hz), 7.63 (1H, dd, J 10.7, 1.9 Hz), 7.48 (1H, d, J 8.5 Hz), 7.09 (1H, t, J 8.7 Hz), 3.80 (2H, t, J 8.3 Hz), 3.53 (2H, dd, J 8.3, 5.5 Hz), 3.34 (2H, t, J 6.2 Hz), 2.88 (2H, d, J 4.9 Hz), 2.70 (2H, s), 2.65-2.61 (1H, m), 1.36 (9H, s), 0.96 (6H, s). LCMS (ES⁺) RT 3.21 minutes, 641 (M+H)⁻.

### Example 62

### 2-(2-Fluoro-4-iodophenylamino)-5,5-dimethyl-8-oxo-5,6,7,8-tetrahydro-4H-thieno[2,3-c]azepine-3-carboxylic acid (azetidin-3-ylmethyl)amide

Prepared from Example 61 (251 mg, 0.39 mmol) and trifluoroacetic acid by the method of Example 56. The title compound was obtained as a yellow solid (9 mg, 4%). δ_{H} (DMSO-d6) 8.38 (1H, s), 7.67 (1H, s), 7.58 (1H, dd, J 10.5, 1.7 Hz), 7.48 (1H, dd, J 8.5, 1.1 Hz), 7.15 (1H, t, J 8.8 Hz), 3.49-3.42 (4H, m), 3.15 (2H, dd, J 13.0, 8.4 Hz), 2.85 (2H, d, J 4.7 Hz), 2.66 (2H, s), 2.08-1.95 (1H, m), 1.23-1.15 (2H, m), 0.97 (6H, s). LCMS (ES⁺) RT 2.49 minutes, 543 (M+H)⁺.

### Example 63

### 3-[3-(Aminomethyl)azetidin-1-ylcarbonyl]-2-(2-fluoro-4-iodophenylamino)-5,5-dimethyl-4,5,6,7-tetrahydrothieno[2,3-c]azepin-8-one

Prepared from Example 49 (463 mg, 0.72 mmol) and trifluoroacetic acid by the method of Example 56. The title compound was obtained as a yellow solid (254 mg, 65%). δ_{H} (DMSO-d6) 7.78 (1H, t, J 4.8 Hz), 7.64 (1H, dd, J 10.6, 1.8 Hz), 7.45 (1H, d, J=8.5 Hz), 7.00 (1H, t, J 8.7 Hz), 5.26 (2H, br s), 3.91 (2H, t, J 8.7 Hz), 3.62-3.57 (2H, m), 2.85 (2H, d, J 5.0 Hz), 2.71 (2H, d, J 7.0 Hz), 2.56 (2H, s), 0.96 (6H, s). LCMS (ES⁺) RT 2.39 minutes, 543 (M+H)⁺.

### Example 64

### (+)-2-({[2-(2-Fluoro-4-iodophenylamino)-5,5-dimethyl-8-oxo-5,6,7,8-tetrahydro-4H-thieno[2,3-c]azepin-3-ylcarbonyl]amino}methyl)piperidine-1-carboxylic acid tert-butyl ester

Prepared from Intermediate 5 (350 mg, 0.55 mmol) and racemic 2-(aminomethyl)-piperidine-1-carboxylic acid tert-butyl ester (233 mg, 1.09 mmol) by the method of Example 19. The title compound was obtained as a cream solid (304 mg, 83%). δ_{H} (DMSO-d6) 9.31 (1H, br s), 7.93 (1H, t, J 4.7 Hz), 7.85-7.80 (1H, m), 7.64 (1H, dd, J 10.7, 1.9 Hz), 7.50 (1H, d, J 8.5 Hz), 7.15 (1H, t, J 8.7 Hz), 4.28-4.21 (1H, m), 3.83-3.79 (1H, m), 3.42-3.29 (2H, m), 2.88 (3H, d, J 4.9 Hz), 2.73 (2H, d, J 4.0 Hz), 1.58-1.38 (5H, m), 1.30 (10H, s), 0.97 (6H, s). LCMS (ES⁺) RT 3.62 minutes, 671 (M+H)⁺.

### Example 65

### N-(2-{[2-(2-Fluoro-4-iodophenylamino)-5,5-dimethyl-8-oxo-5,6,7,8-tetrahydro-4H-thieno[2,3-c]azepin-3-ylcarbonyl]amino}ethyl)-N-methylcarbamic acid tert-butyl ester

Prepared from Intermediate 5 (350 mg, 0.55 mmol) and N-(2-aminoethyl)-N-methylcarbamic acid tert-butyl ester (142 mg, 1.09 mmol) by the method of Example 19. The title compound was obtained as a cream solid (300 mg, 87%). δ_{H} (DMSO-d6) 9.33-9.20 (1H, m), 7.93-7.90 (2H, m), 7.65 (1H, dd, J 10.7, 1.9 Hz), 7.50 (1H, d, J 8.5 Hz), 7.15 (1H, t, J 8.5 Hz), 3.29-3.24 (4H, m), 2.88 (2H, d, J 4.9 Hz), 2.79 (3H, s), 2.73 (2H, s), 1.33 (9H, s), 0.97 (6H, s). LCMS (ES⁺) RT 3.42 minutes, 631 (M+H)⁺.

### Example 66

### 2-(2-Fluoro-4-iodophenylamino)-5,5-dimethyl-8-oxo-5,6,7,8-tetrahydro-4H-thieno[2,3-c]azepine-3-carboxylic acid 2-(methylamino)ethylamide hydrochloride

Prepared from Example 65 (273 mg, 0.43 mmol) by the method of Example 7 to give the title compound as a yellow solid (196 mg, 80%). δ_{H} (DMSO-d6) 9.37 (1H, s), 8.94 (2H, br s), 8.20 (1H, br s), 7.93 (1H, d, J 0.4 Hz), 7.64 (1H, d, J 10.4 Hz), 7.51 (1H, d, J 8.3 Hz), 7.19 (1H, t, J 8.5 Hz), 3.47 (2H, d, J 4.6 Hz), 2.95-2.88 (4H, m), 2.78 (2H, s), 2.49 (3H, s), 0.97 (6H, s). LCMS (ES⁺) RT 2.42 minutes, 530 (M+H)⁺.

### Example 67

### 2-(2-Fluoro-4-iodophenylamino)-5,5-dimethyl-8-oxo-5,6,7,8-tetrahydro-4H-thieno[2,3-c]azepine-3-carboxylic acid (piperidin-2-ylmethyl)amide hydrochloride

Prepared from Example 64 (284 mg, 0.42 mmol) by method of Example 7 to give the title compound as a yellow solid (157 mg, 66%). δ_{H} (DMSO-d6) 9.24 (1H, s), 9.10 (1H, br s), 8.89-8.86 (1H, m), 8.28 (1H, br s), 7.95 (1H, br s), 7.63 (1H, dd, J 10.7, 1.9 Hz), 7.48 (1H, d, J 8.3 Hz), 7.13 (1H, t, J 8.7 Hz), 4.01-3.90 (1H, m), 3.69-3.51 (1H, m), 3.48-3.36 (1H, m), 3.29-3.17 (1H, m), 3.10-2.98 (1H, m), 2.88 (2H, d, J 4.0 Hz), 2.77-2.73 (2H, m), 1.71-1.64 (4H, m), 1.39-1.23 (2H, m), 0.97 (6H, s). LCMS (ES⁺) RT 2.94 minutes, 571 (M+H)⁺.

### Example 68

### 2-(2-Fluoro-4-iodophenylamino)-5,5-dimethyl-8-oxo-5,6,7,8-tetrahydro-4H-thieno[2,3-c]azepine-3-carboxylic acid [3(R)-1-methylpiperidin-3-yl]amide

Paraformaldehyde (63 mg, 2.10 mmol) was added to a solution of Example 23 (250 mg, 0.42 mmol) and sodium cyanoborohydride (32 mg, 0.51 mmol) in MeOH (5 mL) and the reaction mixture was stirred at r.t. for 18 h. 2M HCl (20 mL) was added to the reaction which was then neutralised with NaOH (10% aq) and extracted with DCM (3×100 mL). The combined organic extracts were dried (Na₂SO₄) and concentrated in vacuo. The crude product was purified by chromatography (silica, 0-10% MeOH in DCM) to give the title compound as a white solid (15 mg, 60%). δ_{H} (DMSO-d6) 8.86 (1H, br s), 7.93 (1H, br s), 7.73 (1H, d, J 7.5 Hz), 7.61 (1H, dd, J 10.7, 1.7 Hz), 7.45-7.42 (1H, m), 7.00 (1H, t, J 8.8 Hz), 3.80-3.77 (1H, m), 2.86 (2H, d, J 5.1 Hz), 2.68 (2H, s), 2.49-2.46 (2H, m), 2.11 (3H, s), 1.95-1.92 (1H, m), 1.89-1.72 (1H, m), 1.60-1.52 (2H, m), 1.48-1.40 (1H, m), 1.25-1.17 (1H, m), 0.96 (6H, d, J 2.0 Hz). LCMS (ES⁺) RT 2.43 minutes, 571 (M+H)⁺.

### Example 69

### 3-(3-Aminoazetidin-1-ylcarbonyl)-2-(2-fluoro-4-iodophenylamino)-5,5-dimethyl-4,5,6,7-tetrahydrothieno[2,3-c]azepin-8-one

Prepared from Intermediate 27 (367 mg, 0.58 mmol) and trifluoroacetic acid by the method of Example 56. The title compound was obtained as a cream solid (215 mg, 70%). δ_{H} (DMSO-d6) 7.86 (1H, t, J 4.9 Hz), 7.64 (1H, dd, J 10.6, 1.9 Hz), 7.48-7.45 (1H, m), 7.04 (1H, t, J 8.7 Hz), 4.02 (2H, t, J 8.5 Hz), 3.65-3.50 (3H, m), 2.85 (2H, d, J=5.0 Hz), 2.56 (2H, s), 0.95 (6H, s). Some exchangeable protons were not observed. LCMS (ES⁺) RT 1.97 minutes, 529 (M+H)⁺.

### Example 70

### 2-(2-Fluoro-4-iodophenylamino)-5,5-dimethyl-8-oxo-5,6,7,8-tetrahydro-4H-thieno[2,3-c]azepine-3-carboxylic acid azetidin-3-ylamide

Prepared from Intermediate 28 (212 mg, 0.33 mmol) and trifluoroacetic acid by the method of Example 19. The title compound was obtained as a cream solid (18 mg, 10%). δ_{H} (DMSO-d6) 7.94 (1H, t, J 4.9 Hz), 7.75 (1H, dd, J 10.7, 1.8 Hz), 7.64 (1H, d, J 8.5 Hz), 7.47 (1H, t, J 8.7 Hz), 4.42 (1H, t, J 8.0 Hz), 4.29-4.16 (2H, m), 2.92 (2H, s), 2.87 (2H, d, J 5.0 Hz), 2.71 (2H, d, J 5.0 Hz), 0.98 (6H, s). Some exchangeable protons were not observed. LCMS (ES⁺) RT 2.05 minutes, 529 (M+H)⁺.

### Example 71

### 3-[3-(Aminomethyl)azetidin-1-ylcarbonyl]-2-(2-fluoro-4-iodophenylamino)-5,5,7-trimethyl-4,5,6,7-tetrahydrothieno[2,3-c]azepin-8-one

Intermediate 32 (240 mg, 0.37 mmol) was dissolved in DCM (5 mL) and TFA (2.5 mL) was added. The reaction mixture was stirred overnight and then quenched by the addition of 10% aqueous NaOH. The DCM was separated and the aqueous layer was extracted with DCM (2×25 mL). The combined organics were dried with MgSO₄ and the solvent was removed under reduced pressure. The resulting oil was purified on silica (DCM DCM/MeOH 2.5%) followed by preparative HPLC to yield the title compound as an off-white solid (9 mg, 6%). δ_{H} (d₆-DMSO) 7.63-7.56 (1H, m), 7.45-7.39 (1H, m), 6.98 (1H, t, J 8.9 Hz), 3.93-3.85 (2H, m), 3.63-3.54 (2H, m), 3.30 (2H, s), 3.06 (2H, s), 3.04 (3H, s), 2.59 (2H, m), 0.98 (6H, s). Some exchangeable protons were not observed. LCMS (ES⁺) RT (pH 3) 2.17 minutes, 557 (M+H)⁺.

### Example 72

### 2-(2-Fluoro-4-iodophenylamino)-3-[3-hydroxy-3-(nitromethyl)azetidin-1-ylcarbonyl]-5,5-dimethyl-4,5,6,7-tetrahydrothieno[2,3-c]azepin-8-one

Intermediate 33 (200 mg, 0.86 mmol) was dissolved in DCM (10 mL) and trifluoroacetic acid (1 mL) was added. The reaction mixture was stirred for 1 h and then the solvent and remaining acid were removed under vacuum at room temperature. The resulting salt was dissolved in DMF (8.3 mL) and Intermediate 5 (353 mg, 0.55 mmol) and triethylamine (317 μL) were added sequentially. The reaction was stirred for 18 h and the DMF was then removed under reduced pressure. A saturated aqueous solution of NaHCO₃ was then added to the pale solid and the biphasic system was stirred for 1 h. The remaining solid was filtered, washed with further NaHCO₃ (aq) and dried. This was then stirred in 50:50 MeCN/isopropyl ether, filtered once more and dried in a vacuum oven at 50° C. to yield the title compound (128 mg, 40%). δ_{H} (d₆-DMSO) 8.71 (1H, s), 7.86 (1H, t, J 5.1 Hz), 7.67 (1H, dd, J 10.5, 1.9 Hz), 7.53-7.47 (1H, m), 7.08 (1H, t, J 8.7 Hz), 6.48 (1H, s), 4.82 (2H, s), 4.16 (2H, d, J 10.2 Hz), 3.87 (2H, d, J 10.2 Hz), 2.86 (2H, d, J 5.1 Hz), 2.59 (2H, s), 0.96 (6H, s). LCMS (ES⁺) RT (pH 10) 2.32 minutes, 589 (M+H)⁺.

### Example 73

### 3-[3-(Aminomethyl)-3-hydroxyazetidin-1-ylcarbonyl]-2-(2-fluoro-4-iodophenylamino)-5,5-dimethyl-4,5,6,7-tetrahydrothieno[2,3-c]azepin-8-one

Intermediate 34 was reacted according to method of Example 72 to yield the title compound (250 mg, 81%). δ_{H} (d₆-DMSO) 7.88 (1H, t, J 5.1 Hz), 7.63 (1H, dd, J 10.5, 1.9 Hz), 7.48-7.43 (1H, m), 7.01 (1H, t, J 8.7 Hz), 5.66 (1H, br s), 4.58 (2H, br s), 3.86 (2H, d, J 9.8 Hz), 3.67 (2H, d, J 9.8 Hz), 2.85 (2H, d, J 5.1 Hz), 2.62 (2H, s), 2.56 (2H, s), 0.96 (6H, s). One exchangeable proton was not observed. LCMS (ES⁺) RT (pH 10) 1.87 minutes, 559 (M+H)⁺.

### Example 74

### 3-({[2-(2-Fluoro-4-iodophenylamino)-5,5-dimethyl-8-oxo-5,6,7,8-tetrahydro-4H-thieno[2,3-c]azepin-3-ylcarbonyl]amino}methyl)-3-hydroxyazetidine-1-carboxylic acid tert-butyl ester

Intermediate 34 (100 mg, 0.50 mmol) was dissolved in DMF (4.5 mL) and

Intermediate 5 (289 mg, 0.45 mmol) and triethylamine (70 μL) were added sequentially. The reaction mixture was stirred overnight and the DMF was removed under reduced pressure. The resulting solid was dissolved in DCM (25 mL) and extracted with aq NaHCO₃ (25 mL), aq citric acid (25 mL) and brine (25 mL). The organic layer was dried with sodium sulfate and the solvent removed to yield a pale solid. This was purified using silica gel chromatography (DCM to DCM/MeOH 5%) to yield a white solid which was triturated with 50:50 MeCN/isopropyl ether and dried to yield the title compound (198 mg, 73%). δ_{H} (d₆-DMSO) 9.06 (1H, s), 8.07-8.03 (1H, m), 7.95-7.90 (1H, m), 7.64 (1H, d, J 12.2 Hz), 7.52-7.48 (1H, m), 7.15 (1H, t, J 9.0 Hz), 5.86 (1H, s), 3.82 (2H, d, J 9.0 Hz), 3.62-3.57 (2H, m), 3.41-3.37 (2H, m), 2.88 (2H, d, J 4.8 Hz), 2.73 (2H, s), 1.35 (9H, s), 0.95 (6H, s). LCMS (ES⁺) RT (pH 10) 2.02 minutes, 659 (M+H)⁺.

### Example 75

### 2-(2-Fluoro-4-iodophenylamino)-5,5-dimethyl-8-oxo-5,6,7,8-tetrahydro-4H-thieno[2,3-c]azepine-3-carboxylic acid (3-hydroxyazetidin-3-ylmethyl)amide

Example 74 (155 mg, 0.24 mmol) was dissolved in DCM (2.4 mL) and TFA (0.25 mL) was added. The reaction was stirred overnight and the solvent and acid were removed. The amine was then purified with silica gel chromatography (DCM to DCM/MeOH 5%) and preparative HPLC to yield the title compound (15 mg, 11%). δ_{H} (d₆-DMSO) 9.09 (1H, s), 7.66-7.62 (1H, m), 7.58 (1H, dd, J 10.5, 1.9 Hz), 7.50-7.45 (1H, m), 7.23 (1H, t, J 8.9 Hz), 6.90 (1H, br s), 3.55-3.47 (4H, m), 3.40 (2H, s), 2.83 (2H, d, J 4.9 Hz), 2.78 (2H, s), 0.95 (6H, s). Some exchangeable protons were not observed. LCMS (ES⁺) RT (pH 10) 2.02 minutes, 559 (M+H)⁺.

### Example 76

### 2-(2-Chloro-4-iodophenylamino)-5,5-dimethyl-8-oxo-5,6,7,8-tetrahydro-4H-thieno[2,3-c]azepine-3-carbonitrile

Intermediate 39 (2.40 g, 7.13 mmol), Intermediate 2 (1.67 g, 7.13 mmol) and cesium carbonate (2.32 g, 7.13 mmol) in DMF (20 mL) were heated at 80° C. for 2 h. The reaction mixture was poured onto ice-water (200 mL) and acidified with 2M aqueous HCl. The resulting orange precipitate was filtered off, washed with water then acetonitrile and dried in vacuo yielding the title compound (1.70 g, 38%). δ_{H} (DMSO-d₆) 9.79 (1H, s), 7.95-7.91 (2H, m), 7.73 (1H, dd, J 8.4 Hz), 7.21 (1H, d, J 8.4 Hz), 2.93 (2H, d, J 5.0 Hz), 2.63 (2H, s), 0.99 (6H, s). LCMS (ES⁺) RT 3.13 minutes, 472/474 (M+H)⁺.

### Example 77

### 2-(2-Chloro-4-cyanophenylamino)-5,5-dimethyl-8-oxo-5,6,7,8-tetrahydro-4H-thieno[2,3-c]azepine-3-carboxylic acid ethyl ester

Intermediate 6 (1 g, 3.55 mmol), 3-chloro-4-fluorobenzonitrile (0.55 g, 3.55 mmol) and cesium carbonate (1.15 g, 3.55 mmol) in DMF (10 mL) were stirred at 65° C. overnight, then partitioned between DCM (200 mL) and water (200 mL). The organic phase was separated, dried over sodium sulphate and concentrated. After azeotroping with heptane to remove residual DMF the product was subjected to column chromatography (SiO₂; DCM/ethyl acetate) to yield the title compound (800 mg, 54%). δ_{H} (DMSO-d₆) 10.98 (1H, s), 8.17 (1H, d, J 1.9 Hz), 8.13 (1H, t, J 5.0 Hz), 7.90 (1H, dd, J 8.6, 1.9 Hz), 7.80 (1H, d, J 8.6 Hz), 4.33 (2H, q, J 7.1 Hz), 2.93 (2H, s), 2.87 (2H, d, J 5.1 Hz), 1.32 (3H, t, J 7.1 Hz), 1.00 (6H, s). LCMS (ES⁺) RT 3.31 minutes, 418/420 (M+H)⁺.

### Example 78

### 2-(2-Chloro-4-iodophenylamino)-5,5-dimethyl-8-oxo-N-propyl-5,6,7,8-tetrahydro-4H-thieno[2,3-c]azepine-3-carboxamidine

Propylamine (140 μL, 1.70 mmol) in toluene (3 mL) was treated with 2M trimethylaluminium in toluene (852 μL, 1.70 mmol) and the reaction mixture stirred at r.t. for 0.5 h. Example 76 (140 mg, 0.34 mmol) in toluene (2 mL) was added and the reaction mixture stirred at reflux for 3.5 h. The reaction was then cooled to r.t. and poured onto a slurry of silica in DCM (6 mL) and methanol (2 mL). After filtering and washing with further DCM/methanol the filtrate was concentrated in vacuo and dried at 50° C. under vacuum yielding the title compound (137 mg, 15%). δ_{H} (DMSO-d₆) 7.90 (1H, br m), 7.71 (1H, s), 7.55 (1H, d, J 8.5 Hz), 7.20 (1H, d, J 8.5 Hz), 3.20-3.10 (2H, brm), 2.86 (2H, br m), 2.78-2.73 (2H, br m), 1.62-1.55 (2H, br m), 0.98 (6H, s), 0.93 (3H, t, J 7.4 Hz). Some exchangeable protons were not observed. LCMS (ES⁺) RT 2.55 minutes, 531/533 (M+H)⁺.

### Example 79

### 2-(2-Chloro-4-iodophenylamino)-3-(4,4-dimethyl-4,5-dihydro-1H-imidazol-2-yl)-5,5-dimethyl-4,5,6,7-tetrahydrothieno[2,3-c]azepin-8-one

From Example 76 (300 mg, 0.64 mmol), 2M trimethylaluminium in toluene (1.6 mL, 3.18 mmol) and 1,2-diamino-2-methylpropane (280 mg, 3.18 mmol) in toluene (5 mL) by the method of Example 78. After pouring onto a mixture of silica (1 g) and 2:1 DCM/methanol (50 mL) and washing with further DCM/methanol the filtrate was concentrated to yield an orange solid (400 mg). The residue was dissolved in DCM and washed with 2M NaOH, dried and concentrated. Chromatography (silica; DCM→1:1 DCM:ethyl acetate) yielded the title compound (35 mg, 10%). δ_{H} (DMSO-d₆) 11.21 (1H, br s), 8.70 (1H, br s), 7.74 (1H, d, J 2.1 Hz), 7.63 (1H, t, J 5.0 Hz), 7.60 (1H, dd, J 8.6, 2.1 Hz), 7.29 (1H, d, J 8.6 Hz), 3.47 (2H, s), 2.86 (2H, d, J 5.0 Hz), 2.76 (2H, s), 1.38 (6H, s), 1.01 (6H, s). LCMS (ES⁺) RT 2.55 minutes, 543/545 (M+H)⁺.

### Example 80

### 2-(2-Fluoro-4-iodophenylamino)-5,5-dimethyl-8-oxo-5,6,7,8-tetrahydro-4H-thieno[2,3-c]azepine-3-carboxylic acid (2-hydroxyethoxy)amide

Example 2 (1.0 g, 2.6 mmol), EDC (362 mg, 2.9 mmol), HOBT (300 mg, 2.9 mmol), NMM (427 mg, 5.2 mmol) and O-(2-hydroxyethyl)hydroxylamine (163 mg, 2.6 mmol) in DMF (10 mL) and DCM (9 mL) were stirred for 48 h. The reaction mixture was poured onto water and extracted with DCM, the unreacted acid removed by filtration and the remaining organic phase washed with 1M aqueous HCl then dried over sodium sulphate and concentrated in vacuo. Chromatography (silica; ethyl acetate) yielded the title compound. δ_{H} (DMSO-d₆) 11.22 (1H, br s), 8.82 (1H, br s), 7.93 (1H, t, J 4.6 Hz), 7.64 (1H, dd, J 10.7, 2.1 Hz), 7.48 (1H, d, J 8.5 Hz), 7.08 (1H, t, J 8.5 Hz), 4.71 (1H, br s), 3.81-3.74 (2H, m), 3.59-3.50 (2H, m), 2.86 (2H, d, J 5.0 Hz), 2.65 (2H, s), 0.97 (6H, s). LCMS (ES⁺) RT 2.85 minutes, 534 (M+H)⁺.

### Example 81

### 2-(2-Chloro-4-iodophenylamino)-N-(2,3-dihydroxypropyl)-5,5-dimethyl-8-oxo-5,6,7,8-tetrahydro-4H-thieno[2,3-c]azepine-3-carboxamidine

From Example 76 (500 mg, 1.06 mmol), 2M trimethylaluminium in toluene (2.65 mL, 5.30 mmol) and (S)-(+)-(2,2-dimethyl-[1,3]dioxolan-4-yl)methylamine (700 μL, 5.30 mmol) in toluene (10 mL) by the method of Example 78. The reaction was worked up by pouring onto 2M aqueous HCl, extracted into ethyl acetate, the aqueous phase basified with sodium hydroxide and the resulting precipitate collected by filtration. After chromatography (silica; 5% methanol/DCM→10% methanol/DCM) the product was further purified by preparative HPLC yielding the title compound (32 mg, 5%). δ_{H} (d₄-MeOH) 8.57 (1H, s), 7.82 (1H, d, J 2.0 Hz), 7.62 (1H, dd, J 8.6, 2.0 Hz), 3.88-3.84 (1H, br s), 3.62-3.55 (2H, m), 3.54-3.36 (2H, m), 3.05 (2H, s), 2.71 (2H, s), 1.09 (6H, s). Exchangeable protons were not observed. LCMS (ES⁺) RT 2.51 minutes, 563/565 (M+H)⁺.

### Example 82

### 2-(2-Chloro-4-iodophenylamino)-5,5-dimethyl-8-oxo-5,6,7,8-tetrahydro-4H-thieno[2,3-c]azepine-3-carboxylic acid amide

Intermediate 40 (300 mg, 0.46 mol) in THF (10 mL) was treated with aqueous ammonia (1 mL) and the mixture stirred at r.t. for 0.5 h. The reaction mixture was diluted with water, extracted with DCM and the aqueous phase filtered, to yield, after washing with methanol and drying, the title compound (108 mg, 68%). δ_{H} (DMSO-d₆) 9.89 (1H, s), 7.96 (1H, t, J 4.9 Hz), 7.83 (1H, d, J 2.0 Hz), 7.67 (1H, dd, J 8.6, 2.0 Hz), 7.47 (2H, br s), 7.27 (1H, d, J 8.6 Hz), 2.90 (2H, d, J 4.9 Hz), 2.82 (2H, s), 0.98 (6H, s). LCMS (ES⁺) RT 2.98 minutes, 490/492 (M+H)⁺.

### Example 83

### 2-(2-Fluoro-4-iodophenylamino)-5,5-dimethyl-8-oxo-5,6,7,8-tetrahydro-4H-thieno[2,3-c]azepine-3-carboxylic acid (2-aminoethoxy)amide

Example 2 (673 mg, 1.42 mmol) and Intermediate 37 (300 mg, 1.7 mmol) in DMF (10 mL) were treated with CDI (300 mg, 1.84 mmol) and the reaction mixture stirred at r.t. for 18 h. The solvent was removed in vacuo, the residue partitioned between DCM and water, dried (sodium sulphate) and concentrated. The resulting oil was chromatographed (SiO₂; DCM/ethyl acetate) then further purified by HPLC at pH 3 (HCO₂H) to give the title compound (150 mg). δ_{H} (DMSO-d₆) 8.25 (1H, s), 7.67 (1H, t, J=4.9 Hz), 7.75 (1H, dd, J 10.7, 1.9 Hz), 7.49 (1H, ddd, J 8.7, 2.1, 1.1 Hz), 7.25 (1H, t, J 8.9 Hz), 3.90 (2H, t, J 4.7 Hz), 2.99 (2H, s), 2.90 (2H, t, J 2.0 Hz), 2.81 (2H, d, J 5.1 Hz), 0.96 (6H, s). Some exchangeable protons were not observed. LCMS (ES⁺) RT 2.14 minutes, 533 (M+H)⁺.

### Example 84

### N-[2-(2-Fluoro-4-iodophenylamino)-5,5-dimethyl-8-oxo-5,6,7,8-tetrahydro-4H-thieno[2,3-c]azepin-3-ylcarbonyl]guanidine

Example 2 (250 mg, 0.42 mmol) and CDI (75 mg, 0.46 mmol) were mixed in DMF (3 mL) and the reaction mixture stirred at ambient temperature for 15 minutes. Guanidine carbonate (76 mg, 0.42 mmol) and triethylamine (60 μL, 0.42 mmol) were added and the reaction mixture stirred at r.t. for 4 h then 100° C. for 1 h. The solvent was removed in vacuo and the product triturated with DCM then purified further by preparative HPLC to give the title compound. δ_{H} (DMSO-d₆) 13.07 (1H, br s), 7.77 (1H, brt, J 4.8 Hz), 7.69 (1H, dd, J 10.5, 2.1 Hz), 7.60 (1H, ddd, J 8.5, 2.1, 1.1 Hz), 7.40 (1H, t, J 8.7 Hz), 3.13 (2H, s), 2.82 (2H, d, J 5.1 Hz), 0.97 (6H, s). Some exchangeable protons were not observed. LCMS (ES⁺) RT 2.62 minutes, 516 (M+H)⁺.

### Example 85

### 2-(2-Fluoro-4-iodophenylamino)-5,5,7-trimethyl-8-oxo-5,6,7,8-tetrahydro-4H-thieno[2,3-c]azepine-3-carboxylic acid pyrrolidin-3-ylamide

Intermediate 38 (190 mg, 0.29 mmol) was dissolved in a minimum of DCM and treated with 2M hydrogen chloride in diethyl ether (5 mL). After stirring at ambient temperature overnight the reaction mixture was concentrated in vacuo to yield the title compound (160 mg, quant.). δ_{H} (DMSO-d₆) 8.96 (2H, br s), 8.38 (1H, d, J 6.8 Hz), 7.62 (1H, dd, J 10.5, 1.7 Hz), 7.46 (1H, d, J 8.7 Hz), 7.04 (1H, t, J 8.7 Hz), 4.42-4.32 (1H, m), 3.38-3.33 (1H, m), 3.25-3.16 (2H, m), 3.10 (2H, s), 3.04 (3H, s), 3.02-2.94 (1H, m), 2.63 (2H, s), 2.12-1.99 (1H, m), 1.82-1.69 (1H, m), 0.99 (6H, s).

### Example 86

### 2-(2-Fluoro-4-iodophenylamino)-5,5,7-trimethyl-8-oxo-5,6,7,8-tetrahydro-4H-thieno[2,3-c]azepine-3-carboxylic acid (2-amino-2-methylpropyl)amide

Intermediate 31 (450 mg, 0.69 mmol) and 1,2-diamino-2-methylpropane (122 mg, 1.38 mmol) in DMF (5 mL) with triethylamine (78 mg, 0.69 mmol) were stirred at r.t. for 18 h. The solvent was removed in vacuo and the residue azeotroped with heptane then partitioned between ethyl acetate and 1M aqueous NaOH solution. The organic phase was separated, dried (sodium sulphate), concentrated and the residue subjected to column chromatography (silica; 0-10% MeOH in DCM) to yield the title compound (210 mg, 55%). δ_{H} (DMSO-d₆) 9.92 (1H, br s), 7.47 (1H, dd, J 10.8, 1.8 Hz), 7.40 (1H, d, J 8.5 Hz), 7.16 (1H, t, J 8.7 Hz), 3.23 (2H, s), 3.03 (2H, s), 3.01 (3H, s), 2.93 (2H, s), 1.11 (6H, s), 0.98 (6H, s). Some exchangeable protons not observed. LCMS (ES⁺) RT 2.85 minutes, 559 (M+H)⁺.

### Example 87

### 2-(2-Fluoro-4-iodophenylamino)-5,5-dimethyl-8-oxo-5,6,7,8-tetrahydro-4H-thieno[2,3-c]azepine-3-carboxylic acid (6-aminopyridin-3-yl)amide

Intermediate 5 (400 mg, 0.63 mmol) and 2,5-diaminopyridine (100 mg, 0.92 mmol) in DMF (5 mL) with triethylamine (88 μL, 0.63 mmol) were stirred at r.t. for 24 h. The solvent was removed in vacuo and the residue azeotroped with heptane then partitioned between 1M aqueous NaOH and ethyl acetate. The organic phase was separated, dried over sodium sulphate and the solvents removed in vacuo to give a crude residue which was purified by HPLC to yield the title compound. δ_{H} (DMSO-d₆) 9.69 (1H, br s), 8.80 (1H, br s), 8.14 (0.4H, s, HCOOH), 8.00 (1H, br s), 7.96-7.86 (1H, br m), 7.59 (1H, dd, J 10.6, 1.9 Hz), 7.49 (1H, br s), 7.46 (1H, d, J 8.4 Hz), 7.10 (1H, t, J 8.8 Hz), 6.40 (1H, d, J 8.9 Hz), 5.74 (2H, s), 2.89 (2H, d, J 5.0 Hz), 2.73 (2H, s), 0.97 (6H, s). LCMS (ES⁺) RT 2.14 minutes, 566 (M+H)⁺.

### Example 88

### 2-(2-Fluoro-4-iodophenylamino)-5,5-dimethyl-8-oxo-5,6,7,8-tetrahydro-4H-thieno[2,3-c]azepine-3-carboxylic acid (6-aminopyridin-2-yl)amide

Intermediate 5 (400 mg, 0.63 mmol) and 2,6-diaminopyridine (100 mg, 0.92 mmol) in DMF (5 mL) with triethylamine (88 μL, 0.63 mmol) were stirred at r.t. for 24 h. The solvent was removed in vacuo and the residue azeotroped with heptane then partitioned between 1M aqueous NaOH and ethyl acetate. The organic phase was separated, dried over sodium sulphate and the solvents removed in vacuo to give a crude residue which was purified by HPLC to yield the title compound. δ_{H} (DMSO-d₆) 7.91 (1H, br s), 7.58 (1H, d, J 10.7 Hz), 7.45 (1H, d, J 8.9 Hz), 7.35 (1H, t, J 7.8 Hz), 7.30-7.20 (1H, m), 7.13 (1H, t, J 8.7 Hz), 6.17 (1H, d, J 7.9 Hz), 5.75 (2H, br s), 2.86 (2H, d, J 4.8 Hz), 2.81-2.72 (2H, br m), 0.97 (6H, s). Some exchangeable protons not observed. LCMS (ES⁺) RT 2.76 minutes, 566 (M+H)⁺.

### Example 89

### 2-(2-Fluoro-4-iodophenylamino)-5,5-dimethyl-8-oxo-5,6,7,8-tetrahydro-4H-thieno[2,3-c]azepine-3-carboxylic acid N-(2,3-dihydroxypropyl)-N-methylamide

Intermediate 5 (300 mg, 0.63 mmol) and 3-(methylamino)propane-1,2-diol (80 mg, 0.76 mmol) in DMF (5 mL) were stirred at r.t. for 4 h. The solvent was removed in vacuo and the resulting oil azeotroped with heptane then partitioned between 1M aqueous NaOH and ethyl acetate. After drying over sodium sulphate and removal of the volatiles in vacuo the residue was purified by HPLC to yield the required product. δ_{H} (CDCl₃) 7.49-7.40 (2H, m), 7.40-7.39 (2.5H, m), 7.12 (0.5H, br s), 6.06-5.97 (1H, m), 4.08-3.96 (1H, m), 3.96-3.79 (0.5H, m), 3.79-3.64 (1.5H, m), 3.64-3.52 (1.5H, m), 3.44-3.32 (2.5H, m), 3.17-3.10 (2H, m), 3.08 (3H, s), 3.04-2.92 (1H, m), 2.71-2.51 (3H, m), 1.10 (3H, s), 1.04 (3H, s). LCMS (ES⁺) RT 1.68 minutes, 562 (M+H)⁺.

### Example 90

### 2-(2-Fluoro-4-iodophenylamino)-3,5,5-trimethyl-4,5,6,7-tetrahydrothieno[2,3-c]azepin-8-one

Diisobutylaluminium hydride in toluene (1.0M, 2.3 mL, 2.3 mmol) was added to a solution of Example 1 (250 mg, 0.46 mmol) in DCM (25 mL) at 0° C. The mixture was allowed to warm to ambient temperature and stirred for 90 h. The crude reaction mixture was treated with saturated aqueous NH₄Cl (125 mL) and an aqueous solution of Rochelle's salt (125 mL) and then extracted with DCM (3×125 mL). The combined organic extracts were washed with brine (200 mL), dried (MgSO₄), filtered and concentrated in vacuo. The crude product was purified by chromatography (silica, 0-25% ethyl acetate in DCM) to give the title compound as a white solid (146 mg, 69%). δ_{H} (DMSO-d6) 8.04 (1H, s), 7.93-7.89 (1H, m), 7.52 (1H, dd, J 10.9, 1.9 Hz), 7.32 (1H, d, J 8.4 Hz), 6.50-6.42 (1H, m), 3.29 (2H, s), 2.86 (2H, q, J 5.3 Hz), 1.85 (3H, s), 0.99 (6H, s). LCMS (ES⁺) RT 3.24 minutes, 445.2 (M+H)⁺.

### Example 91

### 2-(2-Fluoro-4-iodophenylamino)-5,5-dimethyl-8-oxo-5,6,7,8-tetrahydro-4H-thieno[2,3-c]azepine-3-carboxylic acid amide

Example 1 (30 mg, 0.05 mmol) and aqueous ammonia solution (2 mL) was dissolved in 1,4-dioxane (2 mL). The mixture was stirred at 120° C. for 90 minutes in the microwave. The crude reaction was concentrated in vacuo and partitioned between water (100 mL) and DCM. The aqueous phase was extracted with DCM (3×100 mL). The combined organic extracts were washed with brine (100 mL), dried (MgSO₄), filtered and concentrated in vacuo. The crude product was purified by chromatography (silica, 0-35% ethyl acetate in DCM) to give the title compound as a beige solid (5 mg, 19%). δ_{H} (DMSO-d6) 9.89 (1H, s), 8.12-8.05 (1H, m), 7.72 (1H, d, J 10.7 Hz) 7.60 (1H, d, J 8.0 Hz), 7.42-7.32 (1H, m), 7.22 (2H, s), 2.88 (2H, s), 2.68 (2H, d, J 5.4 Hz), 0.95 (6H, s). LCMS (ES⁺) RT 2.93 minutes, 474 (M+H)⁺.

### Example 92

### 2-(2-Fluoro-4-iodophenylamino)-5,5-dimethyl-8-oxo-5,6,7,8-tetrahydro-4H-thieno[2,3-c]azepine-3-carboxylic acid bis(2-hydroxyethyl)amide

Diethanolamine (502 mg, 4.75 mmol) was added to a solution of Intermediate 5 (500 mg, 0.78 mmol) and triethylamine (0.3 ml, 2.15 mmol) in DCM (10 mL). The reaction mixture was stirred at room temperature for 72 h. Water (150 mL) was added to the crude reaction and the mixture extracted with DCM (3×100 mL). The combined organic extracts were washed with brine (100 mL), dried (MgSO₄), filtered and concentrated in vacuo. The crude product was purified by chromatography (silica, 0-5% methanol in DCM) to give the title compound as a white solid (300 mg, 68%). δ_{H} (DMSO-d6) 8.30 (1H, s), 7.90-7.86 (1H, m), 7.59 (1H, dd, J 10.6, 1.8 Hz), 7.42 (1H, d, J 8.5 Hz), 7.04 (1H, t, J 8.7 Hz), 5.03 (1H, br s), 4.74 (1H, br s), 3.60-3.40 (8H, m), 3.28-3.27 (2H, m), 2.89 (2H, br s), 0.94 (6H, s). LCMS (ES⁺) RT 2.30 minutes, 562.2 (M+H)⁺.

### Example 93

### 2-(2-Fluoro-4-iodophenylamino)-3-[2-(hydroxymethyl)piperidin-1-ylcarbonyl]-5,5-dimethyl-4,5,6,7-tetrahydrothieno[2,3-c]azepin-8-one

From Intermediate 5 and 2-piperidinemethanol (350 mg, 2.5 mmol) by the method of Example 92 to give the crude product which was purified by chromatography (silica, 0-20% ethyl acetate in DCM) to give the title compound as a beige solid (114 mg, 42%). LCMS (ES⁺) RT 2.66 minutes, 572.2 (M+H)⁺.

### Example 94

### (R)-{1-[2-(2-Fluoro-4-iodophenylamino)-5,5-dimethyl-8-oxo-5,6,7,8-tetrahydro-4H-thieno[2,3-c]azepin-3-ylcarbonyl]pyrrolidin-3-yl}carbamic acid tert-butyl ester

From Intermediate 5 and (3R)-(+)-3-(tert-butoxycarbonylamino)pyrrolidine (300 mg, 1.6 mmol) by the method of Example 92 to give the crude product which was purified by chromatography (silica, 0-50% ethyl acetate in DCM) to give the title compound as a white solid (468 mg, 93%). δ_{H} (DMSO-d6, 100° C.) 8.14 (1H, s), 7.56-7.51 (2H, m), 7.44 (1H, d, J 8.5 Hz), 7.05-6.95 (1H, m), 6.60 (1H, s), 4.00-3.90 (1H, m), 3.60-3.40 (2H, m), 3.40-3.25 (1H, m), 3.20-3.10 (1H, m), 3.00-2.90 (4H, m), 2.05-1.95 (1H, m), 1.82-1.70 (1H, m), 1.40 (9H, s), 0.98 (6H, s). LCMS (ES⁺) RT 2.97 minutes, 643.2 (M+H)⁺.

### Example 95

### (S)-{1-[2-(2-Fluoro-4-iodophenylamino)-5,5-dimethyl-8-oxo-5,6,7,8-tetrahydro-4H-thieno[2,3-c]azepin-3-ylcarbonyl]pyrrolidin-3-yl}carbamic acid tert-butyl ester

From Intermediate 5 and (3S)-(−)-3-(tert-butoxycarbonylamino)pyrrolidine (300 mg, 1.6 mmol) by the method of Example 92 to give the crude product which was purified by chromatography (silica, 0-50% ethyl acetate in DCM) to give the title compound as a white solid (490 mg, 98%). δ_{H} (DMSO-d6, 100° C.) 8.14 (1H, s), 7.56-7.46 (2H, m), 7.44 (1H, d, J 8.5 Hz), 7.00 (1H, t, J 8.5 Hz), 6.60 (1H, s), 4.00-3.90 (1H, m), 3.58-3.40 (2H, m), 3.38-3.22 (1H, m), 3.16 (1H, dd, J 11.4, 5.2 Hz), 3.10-2.87 (4H, m), 2.05-1.95 (1H, m), 1.85-1.73 (1H, m), 1.40 (9H, s), 1.00 (6H, s). LCMS (ES⁺) RT 3.03 minutes, 643.2 (M+H)⁺.

### Example 96

### (R)-{1-[2-(2-Fluoro-4-iodophenylamino)-5,5-dimethyl-8-oxo-5,6,7,8-tetrahydro-4H-thieno[2,3-c]azepin-3-ylcarbonyl]pyrrolidin-2-ylmethyl}carbamic acid tert-butyl ester

From Intermediate 5 and (R)-pyrrolidin-2-ylmethylcarbamic acid tert-butyl ester (312 mg, 1.56 mmol) by the method of Example 92 to give the crude product which was purified by chromatography (silica, 20-50% ethyl acetate in DCM) to give the title compound as a white solid (482 mg, 94%). δ_{H} (DMSO-d6, 110° C.) 8.06 (1H, s), 7.52 (1H, dd, J 10.5, 1.9 Hz), 7.47 (1H, s), 7.42 (1H, d, J 8.5 Hz), 7.05-7.01 (1H, m), 6.30 (1H, s), 4.10-4.00 (1H, m), 3.40 (2H, s), 3.10-3.05 (1H, m), 3.00-2.95 (4H, m), 2.65-2.50 (1H, m), 1.92-1.65 (4H, m), 1.39 (9H, s), 1.01 (3H, s), 0.98 (3H, s). LCMS (ES⁺) RT 3.15 minutes, 657.2 (M+H)⁺.

### Example 97

### (S)-{1-[2-(2-Fluoro-4-iodophenylamino)-5,5-dimethyl-8-oxo-5,6,7,8-tetrahydro-4H-thieno[2,3-c]azepin-3-ylcarbonyl]pyrrolidin-2-ylmethyl}carbamic acid tert-butyl ester

From Intermediate 5 and (S)-pyrrolidin-2-ylmethylcarbamic acid tert-butyl ester (312 mg, 1.56 mmol) by the method of Example 92 to give the crude product which was purified by chromatography (silica, 20-50% ethyl acetate in DCM) to give the title compound as a white solid (504 mg, 98%). δ_{H} (DMSO-d6, 110° C.) 8.06 (1H, s), 7.52 (1H, dd, J 10.5, 1.8 Hz), 7.47 (1H, s), 7.42 (1H, d, J 8.5 Hz), 7.05-7.01 (1H, m), 6.30 (1H, s), 4.10-4.00 (1H, m), 3.34 (2H, br s), 3.10-3.05 (1H, m), 3.00-2.90 (4H, m), 2.65-2.50 (1H, m), 1.90-1.65 (4H, m), 1.40 (9H, s), 1.01 (3H, s), 0.98 (3H, s). LCMS (ES⁺) RT 3.14 minutes, 657.2 (M+H)⁺.

### Example 98

### 3-[3(R)-3-Aminopyrrolidin-1-ylcarbonyl]-2-(2-fluoro-4-iodophenylamino)-5,5-dimethyl-4,5,6,7-tetrahydrothieno[2,3-c]azepin-8-one hydrochloride

Example 94 (460 mg, 0.7 mmol) in ether (10 mL) was stirred at room temperature with 2N aqueous hydrochloric acid (4.8 ml, 9 mmol) for 65 h. Crude solids were filtered and washed with ether (2×5 mL) and dried to give the title compound as a yellow solid (368 mg, 91%). δ_{H} (DMSO-d6, 100° C.) 8.31 (3H, br s), 7.56 (1H, dd, J 10.4, 1.8 Hz), 7.50-7.40 (2H, m), 7.09 (1H, t, J 8.4 Hz), 3.85-3.73 (1H, m), 3.72-3.55 (2H, m), 3.52-3.40 (3H, m), 2.92 (2H, s), 2.55-2.45 (2H, m), 2.30-2.15 (1H, m), 2.10-1.98 (1H, m), 1.00 (6H, s). LCMS (ES⁺) RT 2.05 minutes, 543.0 (M+H)⁺.

### Example 99

### 3-[3(S)-3-Aminopyrrolidin-1-ylcarbonyl)-2-(2-fluoro-4-iodophenylamino)-5,5-dimethyl-4,5,6,7-tetrahydrothieno[2,3-c]azepin-8-one hydrochloride

Example 95 (506 mg, 0.78 mmol) in diethyl ether (10 mL) was stirred at room temperature with 2N aqueous hydrochloric acid (4.8 mL, 9 mmol) for 65 h. Crude solids were filtered and washed with ether (2×5 mL) and dried to give the title compound as a yellow solid (385 mg, 85%). δ_{H} (DMSO-d6, 110° C.) 8.35 (3H, v br s), 7.56 (1H, dd, J 10.4, 1.8 Hz), 7.50-7.40 (2H, m), 7.15-7.08 (1H, m), 3.80-3.73 (1H, m), 3.72-3.58 (2H, m), 3.57-3.33 (3H, m), 2.92 (2H, s), 2.55-2.50 (2H, m), 2.30-2.15 (1H, m), 2.15-1.98 (1H, m), 1.00 (6H, s). LCMS (ES⁺) RT 1.89 minutes, 543.0 (M+H)⁺.

### Example 100

### 3-[2(R)-2-(Aminomethyl)pyrrolidin-1-ylcarbonyl)-2-(2-fluoro-4-iodophenylamino)-5,5-dimethyl-4,5,6,7-tetrahydrothieno[2,3-c]azepin-8-one hydrochloride

Example 96 (460 mg, 0.70 mmol) in diethyl ether (10 mL) was stirred at room temperature with 2N aqueous hydrochloric acid (9.5 mL, 19 mmol) for 150 h. Crude solids were filtered and washed with ether (2×5 mL) and dried to give the title compound as an off-white solid (350 mg, 84%). δ_{H} (DMSO-d6, 130° C.) 8.07 (3H, br s), 7.55 (1H, dd, J 10.5, 1.7 Hz), 7.46 (1H, d, J 8.6 Hz), 7.41 (1H, br s), 7.09 (1H, t, J 8.6 Hz), 4.25 (1H, m), 3.40 (2H, m), 3.20-2.80 (6H, m), 2.64-2.45 (1H, m), 2.15-2.05 (1H, m), 1.92-1.70 (3H, m), 1.02 (3H, s), 0.99 (3H, s). LCMS (ES⁺) RT 2.02 minutes, 557.2 (M+H)⁺.

### Example 101

### 3-[2(S)-2-(Aminomethyl)pyrrolidin-1-ylcarbonyl)-2-(2-fluoro-4-iodophenylamino)-5,5-dimethyl-4,5,6,7-tetrahydrothieno[2,3-c]azepin-8-one hydrochloride

Example 97 (480 mg, 0.73 mmol) in diethyl ether (10 mL) was stirred at room temperature with 2N aqueous hydrochloric acid (9.5 mL, 19 mmol) for 150 h. Crude solids were filtered and washed with ether (2×5 mL) and dried to give the title compound as a white solid (380 mg, 87%). δ_{H} (DMSO-d6, 130° C.) 8.08 (3H, br s), 7.55 (1H, dd, J 10.5, 1.6 Hz), 7.46 (1H, d, J 8.5 Hz), 7.41 (1H, s), 7.12-7.08 (1H, m), 4.30-4.20 (1H, m), 3.50-3.38 (2H, m), 3.10-2.80 (6H, m), 2.62-2.46 (1H, m), 2.13-2.00 (1H, m), 1.92-1.70 (3H, m), 1.02 (3H, s), 0.99 (3H, s). LCMS (ES⁺) RT 2.01 minutes, 557.2 (M+H)⁺.

### Example 102

### 2-(2-Fluoro-4-iodophenylamino)-5,5-dimethyl-8-oxo-5,6,7,8-tetrahydro-4H-thieno[2,3-c]azepine-3-carboxylic acid (1-methylpyrrolidin-3-yl)amide

Paraformaldehyde (62 mg, 0.48 mmol) and sodium borohydride (35 mg, 0.55 mmol) was added to a solution of Example 60 (168 mg, 0.29 mmol) in methanol (10 mL). The reaction mixture was stirred at room temperature for 18 h. The pH was adjusted to 3 with the addition of aqueous 2N HCl, then basified with 10% aqueous NaOH solution and extracted with DCM (3×100 mL). The combined organic extracts were washed with brine (100 mL), dried (MgSO₄), filtered and concentrated in vacuo. The crude product was purified by chromatography (silica, 0-15% methanol in DCM) to give the title compound as an off-white solid (148 mg, 92%). δ_{H} (DMSO-d6) 8.85 (1H, br s), 8.12-8.05 (1H, m), 8.00-7.90 (1H, m), 7.60 (1H, d, J 10.6 Hz), 7.43 (1H, d, J 8.3 Hz), 7.00 (1H, t, J=8.8 Hz), 4.30-4.12 (1H, m), 2.85 (2H, d, J 4.7 Hz), 2.75-2.60 (3H, m), 2.55-2.35 (2H, m), 2.30-2.15 (4H, m), 2.12-1.97 (1H, m), 1.70-1.50 (1H, m), 0.95 (6H, s). LCMS (ES⁺) RT 1.98 minutes, 557.4 (M+H)⁺.

### Example 103

### 2(S)-2-tert-Butoxycarbonylamino-3-{[2-(2-fluoro-4-iodophenylamino)-5,5-dimethyl-8-oxo-5,6,7,8-tetrahydro-4H-thieno[2,3-c]azepin-3-ylcarbonyl]amino}propionic acid

tert-Butoxycarbonyl-DAP-OH (188 mg, 0.92 mmol) was added to a solution of Intermediate 5 (506 mg, 0.78 mmol) and triethylamine (0.13 mL, 0.92 mmol) in DCM (8 mL). The mixture was stirred at room temperature for 48 h. H₂O (75 mL) was added followed by 2 mL of citric acid (sat aqueous solution) and extracted with DCM/EtOAc (75:25) (3×100 mL). The combined organic extracts were washed with brine (100 mL), dried (MgSO₄), filtered and concentrated in vacuo. The crude product was triturated with DCM (5 mL) to give the title compound as a white solid (225 mg, 74%). δ_{H} (DMSO-d6) 12.65 (1H, br s), 9.24 (1H, br s), 8.00-7.87 (2H, m), 7.65 (1H, dd, J 10.5, 1.8 Hz), 7.52 (1H, d, J 8.5 Hz), 7.22 (1H, t, J 8.7 Hz), 7.00 (1H, d, J 7.8 Hz), 4.20-4.08 (1H, m), 3.60-3.40 (2H, m), 2.87 (2H, d, J 4.8 Hz), 2.73 (2H, s), 1.36 (9H, s), 0.96 (6H, s). LCMS (ES⁺) RT 2.74 minutes, 659.2 (M)⁺.

### Example 104

### 2(S)-2-Amino-3-{[2-(2-fluoro-4-iodophenylamino)-5,5-dimethyl-8-oxo-5,6,7,8-tetrahydro-4H-thieno[2,3-c]azepin-3-ylcarbonyl]amino}propionic acid hydrochloride

Example 103 (205 mg, 0.31 mmol) in tetrahydrofuran (8 mL) was stirred at room temperature with 5N aqueous hydrochloric acid for 18 h. Crude solids were filtered and dried to give the title compound as an off-white solid (115 mg, 62%). δ_{H} (DMSO-d6) 13.86 (1H, br s), 9.36 (1H, br s), 8.39 (3H, br s), 8.20-8.10 (1H, m), 7.98-7.88 (1H, m), 7.67 (1H, dd, J 10.6, 1.5 Hz), 7.53 (1H, d, J 8.5 Hz), 7.30-7.20 (1H, m), 4.10-4.00 (1H, m), 3.80-3.60 (2H, m), 2.89 (2H, d, J 4.2 Hz), 2.78 (2H, s), 0.97 (6H, s). LCMS (ES⁺) RT 2.08 minutes, 561.1 (M+H)⁺.

### Example 105

### 2-(2-Fluoro-4-iodophenylamino)-3-[3(S)-3-(hydroxymethyl)morpholin-4-ylcarbonyl]-5,5-dimethyl-4,5,6,7-tetrahydrothieno[2,3-c]azepin-8-one

(S)-Morpholin-3-ylmethanol (417 mg, 3.56 mmol) was added to a solution of Intermediate 5 (300 mg, 0.47 mmol) and triethylamine (0.33 mL, 2.38 mmol) in DCM (7 mL). The mixture was stirred at room temperature for 48 h. H₂O (75 mL) was added and the mixture extracted with DCM (3×100 mL). The combined organic extracts were washed with brine (100 mL), dried (MgSO₄), filtered and concentrated in vacuo. The crude product was triturated with DCM (5 mL) to give the title compound as a white solid (115 mg, 43%). LCMS (ES⁺) RT 2.42 minutes, 574.1 (M+H)⁺.

### Example 106

### (N-{Ethoxycarbonylmethyl}-N-13(R)-1-[2-(2-fluoro-4-iodophenylamino)-5,5-dimethyl-8-oxo-5,6,7,8-tetrahydro-4H-thieno[2,3-c]azepin-3-ylcarbonyl]pyrrolidin-3-yl}amino)acetic acid ethyl ester

{N-(Ethoxycarbonylmethyl)-N-[3(R)-pyrrolidin-3-yl]amino}acetic acid ethyl ester (82 mg, 0.28 mmol) was added to a solution of Intermediate 5 (100 mg, 0.15 mmol) and triethylamine (0.15 mL, 1.08 mmol) in DCM (7 mL). The mixture was stirred at room temperature for 120 h. H₂O (75 mL) was added and the pH was adjusted to 4 using aqueous citric acid solution. The mixture was extracted with DCM (3×100 mL). The combined organic extracts were washed with brine (100 mL), dried (MgSO₄), filtered and concentrated in vacuo. The crude product was purified by chromatography (silica, 0-50% ethyl acetate in DCM) to give the title compound as a white solid (105 mg, 98%). δ_{H} (CDCl₃) 7.50-7.38 (2H, m), 7.35-7.28 (2H, m), 6.43-6.30 (1H, m), 4.30-4.10 (4H, m), 3.90-3.20 (9H, m), 3.00 (2H, br s), 2.75-2.50 (2H, m), 2.14-2.05 (1H, m), 2.00-1.80 (1H, m), 1.40-1.20 (6H, m), 1.07 (3H, s), 1.04 (3H, s). LCMS (ES⁺) RT 2.75 minutes, 715.0 (M+H)⁺.

### Example 107

### 2-(2-Fluoro-4-iodophenylamino)-5,5-dimethyl-8-oxo-5,6,7,8-tetrahydro-4H-thieno[2,3-c]azepine-3-carboxylic acid piperidin-2(R)-ylmethyl ester

(R)-Piperidin-2-ylmethanol hydrochloride salt (237 mg, 1.56 mmol) was added to a solution of Intermediate 5 (500 mg, 0.78 mmol) and triethylamine (0.3 mL, 1.71 mmol) in DCM (8 mL). The mixture was stirred at room temperature for 18 h and later heated at 50° C. for 24 h. H₂O (75 mL) was added and the pH was adjusted to 4 using aqueous citric acid solution. The mixture was extracted with DCM (3×100 ml). The combined organic extracts were washed with brine (100 mL), dried (MgSO₄), filtered and concentrated in vacuo. The crude product was purified by chromatography (silica, 0-2% methanol in DCM) to give the title compound as a white solid (200 mg, 45%). 8H (DMSO-d6) 7.92 (1H, t, J 4.7 Hz), 7.80 (1H, dd, J 10.2, 1.9 Hz), 7.64 (1H, d, J 8.4 Hz), 7.42 (1H, t, J 8.5 Hz), 4.12 (2H, dq, J 10.8, 4.6 Hz), 3.40-3.12 (3H, m), 2.92 (2H, s), 2.87 (2H, d, J 5.1 Hz), 2.84-2.75 (1H, m), 2.55-2.45 (1H, m), 1.80-1.70 (1H, m), 1.70-1.58 (1H, m), 1.54-1.40 (1H, m), 1.40-1.10 (3H, m), 0.98 (6H, s). LCMS (ES⁺) RT 2.07 minutes, 572 (M+H)⁺.

### Example 108

### 2-(2-Fluoro-4-iodophenylamino)-5,5-dimethyl-8-oxo-5,6,7,8-tetrahydro-4H-thieno[2,3-c]azepine-3-carboxylic acid piperidin-2(S)-ylmethyl ester

(S)-Piperidin-2-ylmethanol hydrochloride salt (240 mg, 1.56 mmol) was added to a solution of Intermediate 5 (510 mg, 0.78 mmol) and triethylamine (0.3 mL, 1.71 mmol) in DCM (8 mL). The mixture was stirred at room temperature for 18 h and later heated at 50° C. for 24 h. H₂O (75 mL) was added and the pH adjusted to 4 with aqueous citric acid solution. The mixture was extracted with DCM (3×100 mL) and the combined organic extracts were washed with brine (100 mL), dried (MgSO₄), filtered and concentrated in vacuo. The crude product was purified by chromatography (silica, 0-2% methanol in DCM) to give the title compound as a white solid (200 mg, 45%). δ_{H} (DMSO-d6) 7.92-7.82 (1H, m), 7.76 (1H, d, J 10.0 Hz), 7.62 (1H, d, J 8.2 Hz), 7.39 (1H, t, J 8.5 Hz), 4.09 (2H, dq, J 10.7, 4.4 Hz), 3.40-3.12 (3H, m), 2.91 (2H, s), 2.86 (2H, d, J 5.0 Hz), 2.83-2.71 (1H, m), 2.50-2.40 (1H, m), 1.80-1.70 (1H, m), 1.68-1.58 (1H, m), 1.56-1.42 (1H, m), 1.40-1.10 (3H, m), 0.97 (6H, s). LCMS (ES⁺) RT 2.05 minutes, 572.2 (M+H)⁺.

### Example 109

### (S)-4-[2-(2-Fluoro-4-iodophenylamino)-5,5-dimethyl-8-oxo-5,6,7,8-tetrahydro-4H-thieno[2,3-c]azepin-3-ylcarbonyl]piperazine-1,3-dicarboxylic acid 1-tert-butyl ester triethylamine salt

(S)-4-N-tert-Butoxycarbonylpiperazine-2-carboxylic acid (336 mg, 1.45 mmol) was added to a solution of Intermediate 5 (300 mg, 0.46 mmol) and triethylamine (0.33 mL, 2.3 mmol) in DCM (8 mL). The mixture was stirred at room temperature for 18 h. Water (75 mL) was added and the mixture extracted with DCM (3×100 mL). The combined organic extracts were washed with brine (100 mL), dried (MgSO₄), filtered and concentrated in vacuo. The crude product was purified by chromatography (silica, 0-10% methanol in DCM) to give the title compound as a white solid (285 mg, 78%). LCMS (ES⁺) RT 2.72 minutes, 687.1 (M+H)⁺.

### Example 110

### (R)-4-[2-(2-Fluoro-4-iodophenylamino)-5,5-dimethyl-8-oxo-5,6,7,8-tetrahydro-4H-thieno[2,3-c]azepin-3-ylcarbonyl]piperazine-1,3-dicarboxylic acid 1-tert-butyl ester triethylamine salt

(R)-4-N-tert-Butoxycarbonylpiperazine-2-carboxylic acid (346 mg, 1.50 mmol) was added to a solution of Intermediate 5 (300 mg, 0.46 mmol) and triethylamine (0.33 mL, 2.3 mmol) in DCM (8 mL). The mixture was stirred at room temperature for 18 h. Water (75 mL) was added and the mixture extracted with DCM (3×100 mL). The combined organic extracts were washed with brine (100 mL), dried (MgSO₄), filtered and concentrated in vacuo. The crude product was purified by chromatography (silica, 0-10% methanol in DCM) to give the title compound as a white solid (266 mg, 74%). LCMS (ES⁺) RT 2.72 minutes, 687.1 (M+H)⁺.

### Example 111

### 1-[2-(2-Fluoro-4-iodophenylamino)-5,5-dimethyl-8-oxo-5,6,7,8-tetrahydro-4H-thieno[2,3-c]azepin-3-ylcarbonyl]piperazine-2(S)-carboxylic acid hydrochloride salt

Example 109 (285 mg, 0.36 mmol) in tetrahydrofuran (8 mL) was stirred at room temperature with 5N aqueous hydrochloric acid for 18 h. Crude solids were filtered and dried to give the title compound as a yellow solid (199 mg, 80%). LCMS (ES⁺) RT 1.45 minutes, 587.1 (M+H)⁺.

### Example 112

### 1-[2-(2-Fluoro-4-iodophenylamino)-5,5-dimethyl-8-oxo-5,6,7,8-tetrahydro-4H-thieno[2,3-c]azepin-3-ylcarbonyl]piperazine-2(R)-carboxylic acid hydrochloride salt

Example 110 (266 mg, 0.34 mmol) in tetrahydrofuran (8 mL) was stirred at room temperature with 5N aqueous hydrochloric acid for 18 h. Crude solids were filtered and dried to give the title compound as a yellow solid (160 mg, 68%). LCMS (ES⁺) RT 1.60 minutes, 587.1 (M+H)⁺.

### Examples 113 to 206

### General Method 1

To Intermediate 41 (35 mg, 1 eq) was added a solution of the desired amine (0.8 eq) in THF/DMF (1:1, 2 mL). The mixture was stirred overnight at room temperature. The resin was then filtered and rinsed with a solution of THF/DMF (1:1). The organic solutions were combined and the volatiles removed in vacuo to give the desired products as crude solids. The compounds were purified by preparative HPLC and characterized by LCMS.

Examples 113 to 206 were all prepared by this method.

### Example 113

### 2-(2-Fluoro-4-iodophenylamino)-5,5-dimethyl-8-oxo-5,6,7,8-tetrahydro-4H-thieno[2,3-c]azepine-3-carboxylic acid N-methyl-N-[2-(methylamino)ethyl]amide

LCMS (ES⁺) RT 1.70 minutes, 545 (M+H)⁺.

### Example 114

### 2-(2-Fluoro-4-iodophenylamino)-5,5-dimethyl-8-oxo-5,6,7,8-tetrahydro-4H-thieno[2,3-c]azepine-3-carboxylic acid (2-hydroxyethyl)amide

LCMS (ES⁺) RT 1.77 minutes, 518 (M+H)⁺.

### Example 115

### 2-(2-Fluoro-4-iodophenylamino)-3-(4-hydroxypiperidin-1-ylcarbonyl)-5,5-dimethyl-4,5,6,7-tetrahydrothieno[2,3-c]azepin-8-one

LCMS (ES⁺) RT 1.85 minutes, 541 (M+H)⁺.

### Example 116

### 2-(2-Fluoro-4-iodophenylamino)-3-(3-hydroxypyrrolidin-1-ylcarbonyl)-5,5-dimethyl-4,5,6,7-tetrahydrothieno[2,3-c]azepin-8-one

LCMS (ES⁺) RT 1.65 minutes, 544 (M+H)⁺.

### Example 117

### 2-(2-Fluoro-4-iodophenylamino)-5,5-dimethyl-8-oxo-5,6,7,8-tetrahydro-4H-thieno[2,3-c]azepine-3-carboxylic acid (3-hydroxypropyl)amide

LCMS (ES⁺) RT 1.80 minutes, 532 (M+H)⁺.

### Example 118

### 2-(2-Fluoro-4-iodophenylamino)-5,5-dimethyl-8-oxo-5,6,7,8-tetrahydro-4H-thieno[2,3-c]azepine-3-carboxylic acid (2-hydroxypropyl)amide

LCMS (ES⁺) RT 1.88 minutes, 532 (M+H)⁺.

### Example 119

### 2-(2-Fluoro-4-iodophenylamino)-5,5-dimethyl-8-oxo-5,6,7,8-tetrahydro-4H-thieno[2,3-c]azepine-3-carboxylic acid [3-(2-oxopyrrolidin-1-yl)propyl]amide

LCMS (ES⁺) RT 1.89 minutes, 559 (M+H)⁺.

### Example 120

### 2-(2-Fluoro-4-iodophenylamino)-3-[4-(2-hydroxyethyl)piperazin-1-ylcarbonyl]-5,5-dimethyl-4,5,6,7-tetrahydrothieno[2,3-c]azepin-8-one

LCMS (ES⁺) RT 1.65 minutes, 587 (M+H)⁺.

### Example 121

### 3-{[2-(2-Fluoro-4-iodophenylamino)-5,5-dimethyl-8-oxo-5,6,7,8-tetrahydro-4H-thieno[2,3-c]azepin-3-ylcarbonyl]amino}propionic acid ethyl ester

LCMS (ES⁺) RT 2.15 minutes, 574 (M+H)⁺.

### Example 122

### 2-(2-Fluoro-4-iodophenylamino)-5,5-dimethyl-8-oxo-5,6,7,8-tetrahydro-4H-thieno[2,3-c]azepine-3-carboxylic acid [2-(dimethylamino)ethyl]amide

LCMS (ES⁺) RT 2.03 minutes, 545 (M+H)⁺.

### Example 123

### 2-(2-Fluoro-4-iodophenylamino)-5,5-dimethyl-8-oxo-5,6,7,8-tetrahydro-4H-thieno[2,3-c]azepine-3-carboxylic acid (1-hydroxycyclohexylmethyl)amide

LCMS (ES⁺) RT 2.23 minutes, 586 (M+H)⁺.

### Example 124

### 2-(2-Fluoro-4-iodophenylamino)-5,5-dimethyl-8-oxo-5,6,7,8-tetrahydro-4H-thieno[2,3-c]azepine-3-carboxylic acid [3-(dimethylamino)-2,2-dimethylpropyl)amide

LCMS (ES⁺) RT 2.39 minutes, 587 (M+H)⁺.

### Example 125

### 2-(2-Fluoro-4-iodophenylamino)-5,5-dimethyl-8-oxo-5,6,7,8-tetrahydro-4H-thieno[2,3-c]azepine-3-carboxylic acid [2-(1-methylpyrrolidin-2-yl)ethyl]amide

LCMS (ES⁺) RT 1.85 minutes, 585 (M+H)⁺.

### Example 126

### 2-(2-Fluoro-4-iodophenylamino)-5,5-dimethyl-8-oxo-5,6,7,8-tetrahydro-4H-thieno[2,3-c]azepine-3-carboxylic acid (pyridin-2-ylmethyl)amide

LCMS (ES⁺) RT 2.05 minutes, 565 (M+H)⁺.

### Example 127

### 2-(2-Fluoro-4-iodophenylamino)-5,5-dimethyl-8-oxo-5,6,7,8-tetrahydro-4H-thieno[2,3-c]azepine-3-carboxylic acid [2-(pyridin-2-yl)ethyl]amide

LCMS (ES⁺) RT 2.07 minutes, 579 (M+H)⁺.

### Example 128

### 2-(2-Fluoro-4-iodophenylamino)-5,5-dimethyl-8-oxo-5,6,7,8-tetrahydro-4H-thieno[2,3-c]azepine-3-carboxylic acid (2-methoxy-1-methylethyl)amide

LCMS (ES⁺) RT 2.14 minutes, 546 (M+H)⁺.

### Example 129

### 2-(2-Fluoro-4-iodophenylamino)-5,5-dimethyl-8-oxo-5,6,7,8-tetrahydro-4H-thieno[2,3-c]azepine-3-carboxylic acid [2-(piperidin-1-yl)ethyl]amide

LCMS (ES⁺) RT 2.30 minutes, 585 (M+H)⁺.

### Example 130

### 2-(2-Fluoro-4-iodophenylamino)-5,5-dimethyl-8-oxo-5,6,7,8-tetrahydro-4H-thieno[2,3-c]azepine-3-carboxylic acid (pyridin-4-ylmethyl)amide

LCMS (ES⁺) RT 1.90 minutes, 565 (M+H)⁺.

### Example 131

### 2-(2-Fluoro-4-iodophenylamino)-5,5-dimethyl-8-oxo-5,6,7,8-tetrahydro-4H-thieno[2,3-c]azepine-3-carboxylic acid [2-(pyrrolidin-1-yl)ethyl]amide

LCMS (ES⁺) RT 2.09 minutes, 571 (M+H)⁺.

### Example 132

### 2-(2-Fluoro-4-iodophenylamino)-5,5-dimethyl-8-oxo-5,6,7,8-tetrahydro-4H-thieno[2,3-c]azepine-3-carboxylic acid (tetrahydrofuran-2-ylmethyl)amide

LCMS (ES⁺) RT 2.12 minutes, 558 (M+H)⁺.

### Example 133

### 2-(2-Fluoro-4-iodophenylamino)-5,5-dimethyl-8-oxo-5,6,7,8-tetrahydro-4H-thieno[2,3-c]azepine-3-carboxylic acid [2-(acetylamino)ethyl]amide

LCMS (ES⁺) RT 1.75 minutes, 559 (M+H)⁺.

### Example 134

### 3-{[2-(2-Fluoro-4-iodophenylamino)-5,5-dimethyl-8-oxo-5,6,7,8-tetrahydro-4H-thieno[2,3-c]azepin-3-ylcarbonyl]amino/butyric acid ethyl ester

LCMS (ES⁺) RT 2.22 minutes, 558 (M+H)⁺.

### Example 135

### 2-(2-Fluoro-4-iodophenylamino)-5,5-dimethyl-8-oxo-5,6,7,8-tetrahydro-4H-thieno[2,3-c]azepine-3-carboxylic acid (2-methoxyethyl)amide

LCMS (ES⁺) RT 2.04 minutes, 532 (M+H)⁺.

### Example 136

### 1-(2-{[2-(2-Fluoro-4-iodophenylamino)-5,5-dimethyl-8-oxo-5,6,7,8-tetrahydro-4H-thieno[2,3-c]azepin-3-ylcarbonyl]amino}-3-methylbutyryl)pyrrolidine-2-carboxylic acid

LCMS (ES⁺) RT 1.66 minutes, 671 (M+H)⁺.

### Example 137

### 2-(2-Fluoro-4-iodophenylamino)-5,5-dimethyl-8-oxo-5,6,7,8-tetrahydro-4H-thieno[2,3-c]azepine-3-carboxylic acid 2-methoxybenzylamide

LCMS (ES⁺) RT 2.35 minutes, 594 (M+H)⁺.

### Example 138

### 2-(2-Fluoro-4-iodophenylamino)-5,5-dimethyl-8-oxo-5,6,7,8-tetrahydro-4H-thieno[2,3-c]azepine-3-carboxylic acid (3-isopropoxypropyl)amide

LCMS (ES⁺) RT 2.27 minutes, 574 (M+H)⁺.

### Example 139

### 2-(2-Fluoro-4-iodophenylamino)-5,5-dimethyl-8-oxo-5,6,7,8-tetrahydro-4H-thieno[2,3-c]azepine-3-carboxylic acid 3-methoxybenzylamide

LCMS (ES⁺) RT 2.28 minutes, 594 (M+H)⁺.

### Example 140

### 2-(2-Fluoro-4-iodophenylamino)-5,5-dimethyl-8-oxo-5,6,7,8-tetrahydro-4H-thieno[2,3-c]azepine-3-carboxylic acid (2-hydroxy-2-phenylethyl)amide

LCMS (ES⁺) RT 2.12 minutes, 594 (M+H)⁺.

### Example 141

### 2-(2-Fluoro-4-iodophenylamino)-5,5-dimethyl-8-oxo-5,6,7,8-tetrahydro-4H-thieno[2,3-c]azepine-3-carboxylic acid [3-(dimethylamino)propyl]amide

LCMS (ES⁺) RT 1.82 minutes, 559 (M+H)⁺.

### Example 142

### 2-(2-Fluoro-4-iodophenylamino)-5,5-dimethyl-8-oxo-5,6,7,8-tetrahydro-4H-thieno[2,3-c]azepine-3-carboxylic acid (3-amino-2,2-dimethylpropyl)amide

LCMS (ES⁺) RT 1.78 minutes, 559 (M+H)⁺.

### Example 143

### 2-(2-Fluoro-4-iodophenylamino)-5,5-dimethyl-8-oxo-5,6,7,8-tetrahydro-4H-thieno[2,3-c]azepine-3-carboxylic acid N-methyl-N-[3-(methylamino)propyl]amide

LCMS (ES⁺) RT 1.58 minutes, 559 (M+H)⁺.

### Example 144

### 2-(2-Fluoro-4-iodophenylamino)-5,5-dimethyl-8-oxo-5,6,7,8-tetrahydro-4H-thieno[2,3-c]azepine-3-carboxylic acid (2-oxotetrahydrofuran-3-yl)amide

LCMS (ES⁺) RT 1.92 minutes, 558 (M+H)⁺.

### Example 145

### 2-(2-Fluoro-4-iodophenylamino)-5,5-dimethyl-8-oxo-5,6,7,8-tetrahydro-4H-thieno[2,3-c]azepine-3-carboxylic acid (2-ethylsulfanylethyl)amide

LCMS (ES⁺) RT 2.28 minutes, 562 (M+H)⁺.

### Example 146

### 2-(2-Fluoro-4-iodophenylamino)-5,5-dimethyl-8-oxo-5,6,7,8-tetrahydro-4H-thieno[2,3-c]azepine-3-carboxylic acid [2-(pyridin-2-ylamino)ethyl]amide

LCMS (ES⁺) RT 2.03 minutes, 594 (M+H)⁺.

### Example 147

### 2-(2-Fluoro-4-iodophenylamino)-5,5-dimethyl-8-oxo-5,6,7,8-tetrahydro-4H-thieno[2,3-c]azepine-3-carboxylic acid (carbamoylmethyl)amide

LCMS (ES⁺) RT 1.69 minutes, 531 (M+H)⁺.

### Example 148

### 2-(2-Fluoro-4-iodophenylamino)-5,5-dimethyl-3-(3-oxopiperazin-1-ylcarbonyl)-4,5,6,7-tetrahydrothieno[2,3-c]azepin-8-one

LCMS (ES⁺) RT 1.61 minutes, 557 (M+H)⁺.

### Example 149

### 3-(4-Acetylpiperazin-1-ylcarbonyl)-2-(2-fluoro-4-iodophenylamino)-5,5-dimethyl-4,5,6,7-tetrahydrothieno[2,3-c]azepin-8-one

LCMS (ES⁺) RT 1.70 minutes, 585 (M+H)⁺.

### Example 150

### 2-(2-Fluoro-4-iodophenylamino)-5,5-dimethyl-8-oxo-5,6,7,8-tetrahydro-4H-thieno[2,3-c]azepine-3-carboxylic acid (2-oxo-2-phenylethyl)amide

LCMS (ES⁺) RT 2.31 minutes, 592 (M+H)⁺.

### Example 151

### 1-[2-(2-Fluoro-4-iodophenylamino)-5,5-dimethyl-8-oxo-5,6,7,8-tetrahydro-4H-thieno[2,3-c]azepin-3-ylcarbonyl]piperidine-4-carboxylic acid amide

LCMS (ES⁺) RT 1.61 minutes, 585 (M+H)⁺.

### Example 152

### 2-(2-Fluoro-4-iodophenylamino)-5,5-dimethyl-8-oxo-5,6,7,8-tetrahydro-4H-thieno[2,3-c]azepine-3-carboxylic acid (pyridin-3-ylmethyl)amide

LCMS (ES⁺) RT 1.92 minutes, 565 (M+H)⁺.

### Example 153

### 2-(2-Fluoro-4-iodophenylamino)-5,5-dimethyl-8-oxo-5,6,7,8-tetrahydro-4H-thieno[2,3-c]azepine-3-carboxylic acid [2-(pyridin-3-yl)ethyl]amide

LCMS (ES⁺) RT 1.97 minutes, 579 (M+H)⁺.

### Example 154

### 2-(2-Fluoro-4-iodophenylamino)-3-[4-(hydroxymethyl)piperidin-1-ylcarbonyl]-5,5-dimethyl-4,5,6,7-tetrahydrothieno[2,3-c]azepin-8-one

LCMS (ES⁺) RT 1.74 minutes, 572 (M+H)⁺.

### Example 155

### 3-(4-Ethylpiperazin-1-ylcarbonyl)-2-(2-fluoro-4-iodophenylamino)-5,5-dimethyl-4,5,6,7-tetrahydrothieno[2,3-c]azepin-8-one

LCMS (ES⁺) RT 1.92 minutes, 571 (M+H)⁺.

### Example 156

### 2-(2-Fluoro-4-iodophenylamino)-5,5-dimethyl-8-oxo-5,6,7,8-tetrahydro-4H-thieno[2,3-c]azepine-3-carboxylic acid (thiazolidin-4-ylmethyl)amide

LCMS (ES⁺) RT 1.99 minutes, 575 (M+H)⁺.

### Example 157

### 2-(2-Fluoro-4-iodophenylamino)-5,5-dimethyl-8-oxo-5,6,7,8-tetrahydro-4H-thieno[2,3-c]azepine-3-carboxylic acid [2-(2-oxoimidazolidin-1-yl)ethyl]amide

LCMS (ES⁺) RT 1.77 minutes, 586 (M+H)⁺.

### Example 158

### 2-(2-Fluoro-4-iodophenylamino)-5,5-dimethyl-8-oxo-5,6,7,8-tetrahydro-4H-thieno[2,3-c]azepine-3-carboxylic acid (1-azabicyclo[2.2.2]oct-3-yl)amide

LCMS (ES⁺) RT 1.77 minutes, 583 (M+H)⁺.

### Example 159

### 2-(2-Fluoro-4-iodophenylamino)-5,5-dimethyl-8-oxo-5,6,7,8-tetrahydro-4H-thieno[2,3-c]azepine-3-carboxylic acid (1H-benzimidazol-2-ylmethyl)amide

LCMS (ES⁺) RT 1.99 minutes, 604 (M+H)⁺.

### Example 160

### 2-(2-Fluoro-4-iodophenylamino)-5,5-dimethyl-8-oxo-5,6,7,8-tetrahydro-4H-thieno[2,3-c]azepine-3-carboxylic acid (1,2,3,4-tetrahydroquinolin-2-ylmethyl)amide

LCMS (ES⁺) RT 2.39 minutes, 619 (M+H)⁺.

### Example 161

### 2-(2-Fluoro-4-iodophenylamino)-5,5-dimethyl-8-oxo-5,6,7,8-tetrahydro-4H-thieno[2,3-c]azepine-3-carboxylic acid (3,3,3-trifluoro-2-hydroxypropyl)amide

LCMS (ES⁺) RT 2.09 minutes, 586 (M+H)⁺.

### Example 162

### 2-(2-Fluoro-4-iodophenylamino)-3-(3-hydroxypiperidin-1-ylcarbonyl)-5,5-dimethyl-4,5,6,7-tetrahydrothieno[2,3-c]azepin-8-one

LCMS (ES⁺) RT 1.80 minutes, 558 (M+H)⁺.

### Example 163

### 2-(2-Fluoro-4-iodophenylamino)-5,5-dimethyl-8-oxo-5,6,7,8-tetrahydro-4H-thieno[2,3-c]azepine-3-carboxylic acid (3-methyl-3H-imidazol-4-ylmethyl)amide

LCMS (ES⁺) RT 1.80 minutes, 568 (M+H)⁺.

### Example 164

### 2-{[2-(2-Fluoro-4-iodophenylamino)-5,5-dimethyl-8-oxo-5,6,7,8-tetrahydro-4H-thieno[2,3-c]azepin-3-ylcarbonyl]amino}-3-(1H-indol-3-yl)propionic acid

LCMS (ES⁺) RT 1.69 minutes, 661 (M+H)⁺.

### Example 165

### 2-(2-Fluoro-4-iodophenylamino)-5,5-dimethyl-8-oxo-5,6,7,8-tetrahydro-4H-thieno[2,3-c]azepine-3-carboxylic acid [2-([1,2,4]triazol-4-yl)ethyl]amide

LCMS (ES⁺) RT 1.80 minutes, 569 (M+H)⁺.

### Example 166

### 2-{[2-(2-Fluoro-4-iodophenylamino)-5,5-dimethyl-8-oxo-5,6,7,8-tetrahydro-4H-thieno[2,3-c]azepin-3-ylcarbonyl]amino}-5-guanidinopentanoic acid

LCMS (ES⁺) RT 1.45 minutes, 631 (M+H)⁺.

### Example 167

### 2-{[2-(2-Fluoro-4-iodophenylamino)-5,5-dimethyl-8-oxo-5,6,7,8-tetrahydro-4H-thieno[2,3-c]azepin-3-ylcarbonyl]amino}-3-(4-hydroxyphenyl)propionic acid

LCMS (ES⁺) RT 1.55 minutes, 638 (M+H)⁺.

### Example 168

### 2-{[2-(2-Fluoro-4-iodophenylamino)-5,5-dimethyl-8-oxo-5,6,7,8-tetrahydro-4H-thieno[2,3-c]azepin-3-ylcarbonyl]amino}-4-methylsulfanylbutyric acid

LCMS (ES⁺) RT 1.62 minutes, 606 (M+H)⁺.

### Example 169

### 2-(2-Fluoro-4-iodophenylamino)-5,5-dimethyl-8-oxo-5,6,7,8-tetrahydro-4H-thieno[2,3-c]azepine-3-carboxylic acid (1-methylpiperidin-2-ylmethyl)amide

LCMS (ES⁺) RT 2.26 minutes, 585 (M+H)⁺.

### Example 170

### 2-(2-Fluoro-4-iodophenylamino)-5,5-dimethyl-8-oxo-5,6,7,8-tetrahydro-4H-thieno[2,3-c]azepine-3-carboxylic acid (1,1-dioxotetrahydrothien-3-yl)amide

LCMS (ES⁺) RT 1.88 minutes, 592 (M+H)⁺.

### Example 171

### 2-(2-Fluoro-4-iodophenylamino)-5,5-dimethyl-8-oxo-5,6,7,8-tetrahydro-4H-thieno[2,3-c]azepine-3-carboxylic acid (2-methanesulfonylethyl)amide

LCMS (ES⁺) RT 1.84 minutes, 580 (M+H)⁺.

### Example 172

### {[2-(2-Fluoro-4-iodophenylamino)-5,5-dimethyl-8-oxo-5,6,7,8-tetrahydro-4H-thieno[2,3-c]azepin-3-ylcarbonyl]amino}acetic acid

LCMS (ES⁺) RT 1.47 minutes, 532 (M+H)⁺.

### Example 173

### 2-(2-Fluoro-4-iodophenylamino)-5,5-dimethyl-8-oxo-5,6,7,8-tetrahydro-4H-thieno[2,3-c]azepine-3-carboxylic acid [2-(3,5-dimethylpyrazol-1-yl)ethyl]amide

LCMS (ES⁺) RT 2.13 minutes, 596 (M+H)⁺.

### Example 174

### 3-(2,6-Dimethylmorpholine-4-ylcarbonyl)-2-(2-fluoro-4-iodophenylamino)-5,5-dimethyl-4,5,6,7-tetrahydrothieno[2,3-c]azepin-8-one

LCMS (ES⁺) RT 2.08 minutes, 572 (M+H)⁺.

### Example 175

### 2-(2-Fluoro-4-iodophenylamino)-5,5-dimethyl-8-oxo-5,6,7,8-tetrahydro-4H-thieno[2,3-c]azepine-3-carboxylic acid N-(tert-butyl)-N-(2-hydroxyethyl)amide

LCMS (ES⁺) RT 2.41 minutes, 574 (M+H)⁺.

### Example 176

### 2-(2-Fluoro-4-iodophenylamino)-3-[2-(hydroxymethyl)piperidin-1-ylcarbonyl]-5,5-dimethyl-4,5,6,7-tetrahydrothieno[2,3-c]azepin-8-one

LCMS (ES⁺) RT 2.33 minutes, 572 (M+H)⁺.

### Example 177

### 2-(2-Fluoro-4-iodophenylamino)-3-(3-hydroxypyrrolidin-1-ylcarbonyl)-5,5-dimethyl-4,5,6,7-tetrahydrothieno[2,3-c]azepin-8-one

LCMS (ES⁺) RT 1.65 minutes, 544 (M+H)⁺.

### Example 178

### 2-(2-Fluoro-4-iodophenylamino)-3-[2-(2-hydroxyethyl)piperidin-1-ylcarbonyl]-5,5-dimethyl-4,5,6,7-tetrahydrothieno[2,3-c]azepin-8-one

LCMS (ES⁺) RT 2.12 minutes, 586 (M+H)⁺.

### Example 179

### 2-(2-Fluoro-4-iodophenylamino)-3-[2-(hydroxymethyl)pyrrolidin-1-ylcarbonyl]-5,5-dimethyl-4,5,6,7-tetrahydrothieno[2,3-c]azepin-8-one

LCMS (ES⁺) RT 1.81 minutes, 558 (M+H)⁺.

### Example 180

### 1-[2-(2-Fluoro-4-iodophenylamino)-5,5-dimethyl-8-oxo-5,6,7,8-tetrahydro-4H-thieno[2,3-c]azepin-3-ylcarbonyl]piperidine-3-carboxylic acid amide

LCMS (ES⁺) RT 1.70 minutes, 585 (M+H)⁺.

### Example 181

### 2-(2-Fluoro-4-iodophenylamino)-3-[3-(hydroxymethyl)piperidin-1-ylcarbonyl]-5,5-dimethyl-4,5,6,7-tetrahydrothieno[2,3-c]azepin-8-one

LCMS (ES⁺) RT 1.84 minutes, 572 (M+H)⁺.

### Example 182

### 1-[2-(2-Fluoro-4-iodophenylamino)-5,5-dimethyl-8-oxo-5,6,7,8-tetrahydro-4H-thieno[2,3-c]azepin-3-ylcarbonyl]pyrrolidine-2-carboxylic acid methyl ester

LCMS (ES⁺) RT 2.03 minutes, 586 (M+H)⁺.

### Example 183

### 2-(2-Fluoro-4-iodophenylamino)-5,5-dimethyl-8-oxo-5,6,7,8-tetrahydro-4H-thieno[2,3-c]azepine-3-carboxylic acid [2-(phenylamino)ethyl]amide

LCMS (ES⁺) RT 2.28 minutes, 593 (M+H)⁺.

### Example 184

### 2-(2-Fluoro-4-iodophenylamino)-5,5-dimethyl-8-oxo-5,6,7,8-tetrahydro-4H-thieno[2,3-c]azepine-3-carboxylic acid (2-hydroxy-1-phenylethyl)amide

LCMS (ES⁺) RT 2.07 minutes, 594 (M+H)⁺.

### Example 185

### 2-(2-Fluoro-4-iodophenylamino)-3-[4-(2-hydroxyethyl)piperidin-1-ylcarbonyl]-5,5-dimethyl-4,5,6,7-tetrahydrothieno[2,3-c]azepin-8-one

LCMS (ES⁺) RT 1.79 minutes, 586 (M+H)⁺.

### Example 186

### 2-(2-Fluoro-4-iodophenylamino)-5,5-dimethyl-8-oxo-5,6,7,8-tetrahydro-4H-thieno[2,3-c]azepine-3-carboxylic acid N-ethyl-N-[3-(ethylamino)propyl]amide

LCMS (ES⁺) RT 1.70 minutes, 587 (M+H)⁺.

### Example 187

### 2-(2-Fluoro-4-iodophenylamino)-5,5-dimethyl-8-oxo-5,6,7,8-tetrahydro-4H-thieno[2,3-c]azepine-3-carboxylic acid {2-[bis(2-hydroxyethyl)amino]ethyl}amide

LCMS (ES⁺) RT 1.74 minutes, 605 (M+H)⁺.

### Example 188

### 2-(2-Fluoro-4-iodophenylamino)-5,5-dimethyl-8-oxo-5,6,7,8-tetrahydro-4H-thieno[2,3-c]azepine-3-carboxylic acid [4-(aminomethyl)cyclohexylmethyl]amide

LCMS (ES⁺) RT 1.76 minutes, 599 (M+H)⁺.

### Example 189

### 2-(2-Fluoro-4-iodophenylamino)-5,5-dimethyl-3-(1,4,7-triazanonan-1-ylcarbonyl)-4,5,6,7-tetrahydrothieno[2,3-c]azepin-8-one

LCMS (ES⁺) RT 1.49 minutes, 586 (M+H)⁺.

### Example 190

### 2-(2-Fluoro-4-iodophenylamino)-3-[2-(methoxymethyl)pyrrolidin-1-ylcarbonyl]-5,5-dimethyl-4,5,6,7-tetrahydrothieno[2,3-c]azepin-8-one

LCMS (ES⁺) RT 2.08 minutes, 572 (M+H)⁺.

### Example 191

### 2-(2-Fluoro-4-iodophenylamino)-5,5-dimethyl-8-oxo-5,6,7,8-tetrahydro-4H-thieno[2,3-c]azepine-3-carboxylic acid N-[2-(dimethylamino)ethyl]-N-ethylamide

LCMS (ES⁺) RT 2.06 minutes, 573 (M+H)⁺.

### Example 192

### 2-(2-Fluoro-4-iodophenylamino)-5,5-dimethyl-8-oxo-5 6,7,8-tetrahydro-4H-thieno[2,3-c]azepine-3-carboxylic acid [11-(hydroxymethyl)-2,2-dimethylpropyl]amide

LCMS (ES⁺) RT 2.12 minutes, 574 (M+H)⁺.

### Example 193

### N-{1-[2-(2-Fluoro-4-iodophenylamino)-5,5-dimethyl-8-oxo-5,6,7,8-tetrahydro-4H-thieno[2,3-c]azepin-3-ylcarbonyl]pyrrolidin-3-yl}acetamide

LCMS (ES⁺) RT 1.63 minutes, 585 (M+H)⁺.

### Example 194

### 2-(2-Fluoro-4-iodophenylamino)-5,5-dimethyl-8-oxo-5,6,7,8-tetrahydro-4H-thieno[2,3-c]azepine-3-carboxylic acid [1-(carbamoyl)ethyl]amide

LCMS (ES⁺) RT 1.77 minutes, 545 (M+H)⁺.

### Example 195

### 2-(2-Fluoro-4-iodophenylamino)-5,5-dimethyl-8-oxo-5,6,7,8-tetrahydro-4H-thieno[2,3-c]azepine-3-carboxylic acid (2-oxoazepan-3-yl)amide

LCMS (ES⁺) RT 2.05 minutes, 585 (M+H)⁺.

### Example 196

### 2-(2-Fluoro-4-iodophenylamino)-5,5-dimethyl-8-oxo-5,6,7,8-tetrahydro-4H-thieno[2,3-c]azepine-3-carboxylic acid N-(2-cyanoethyl)-N-ethylamide

LCMS (ES⁺) RT 1.95 minutes, 555 (M+H)⁺.

### Example 197

### 2-(2-Fluoro-4-iodophenylamino)-5,5-dimethyl-8-oxo-5,6,7,8-tetrahydro-4H-thieno[2,3-c]azepine-3-carboxylic acid N-methyl-N-{2-[2-(methylamino)ethoxy]ethyl}amide

LCMS (ES⁺) RT 1.62 minutes, 589 (M+H)⁺.

### Example 198

### 3-{[2-(2-Fluoro-4-iodophenylamino)-5,5-dimethyl-8-oxo-5,6,7,8-tetrahydro-4H-thieno[2,3-c]azepin-3-ylcarbonyl]amino}propionic acid

LCMS (ES⁺) RT 1.47 minutes, 546 (M+H)⁺.

### Example 199

### 1-[2-(2-Fluoro-4-iodophenylamino)-5,5-dimethyl-8-oxo-5,6,7,8-tetrahydro-4H-thieno[2,3-c]azepin-3-ylcarbonyl]piperidine-2-carboxylic acid

LCMS (ES⁺) RT 1.50 minutes, 586 (M+H)⁺.

### Example 200

### 1-[2-(2-Fluoro-4-iodophenylamino)-5,5-dimethyl-8-oxo-5,6,7,8-tetrahydro-4H-thieno[2,3-c]azepin-3-ylcarbonyl]pyrrolidine-2-carboxylic acid

LCMS (ES⁺) RT 1.46 minutes, 572 (M+H)⁺.

### Example 201

### 2-(2-Fluoro-4-iodophenylamino)-5,5-dimethyl-8-oxo-5,6,7,8-tetrahydro-4H-thieno[2,3-c]azepine-3-carboxylic acid [3-(aminomethyl)cyclohexylmethyl]amide

LCMS (ES⁺) RT 1.80 minutes, 599 (M+H)⁺.

### Example 202

### 2-(2-Fluoro-4-iodophenylamino)-5,5-dimethyl-8-oxo-5,6,7,8-tetrahydro-4H-thieno[2,3-c]azepine-3-carboxylic acid (1-ethylpyrrolidin-2-ylmethyl)amide

LCMS (ES⁺) RT 2.20 minutes, 585 (M+H)⁺.

### Example 203

### 2-(2-Fluoro-4-iodophenylamino)-5,5-dimethyl-8-oxo-5,6,7,8-tetrahydro-4H-thieno[2,3-c]azepine-3-carboxylic acid [3-(morpholin-4-yl)propyl]amide

LCMS (ES⁺) RT 1.89 minutes, 601 (M+H)⁺.

### Example 204

### 2-(2-Fluoro-4-iodophenylamino)-3-(4-hydroxypiperidin-1-ylcarbonyl)-5,5-dimethyl-4,5,6,7-tetrahydrothieno[2,3-c]azepin-8-one

LCMS (ES⁺) RT 1.70 minutes, 558 (M+H)⁺.

### Example 205

### 2-(2-Fluoro-4-iodophenylamino)-5,5-dimethyl-8-oxo-5,6,7,8-tetrahydro-4H-thieno[2,3-c]azepine-3-carboxylic acid N-(2-hydroxyethyl)-N-methylamide

LCMS (ES⁺) RT 1.70 minutes, 532 (M+H)⁺.

### Example 206

### 2-(2-Fluoro-4-iodophenylamino)-5,5-dimethyl-8-oxo-5,6,7,8-tetrahydro-4H-thieno[2,3-c]azepine-3-carboxylic acid N-(2-hydroxyethyl)-N-propylamide

LCMS (ES⁺) RT 2.35 minutes, 560 (M+H)⁺.

### Example 207

### 2-(2-Fluoro-4-iodophenylamino)-8-imino-5,5-dimethyl-5,6,7,8-tetrahydro-4H-thieno[2,3-c]azepine-3-carboxylic acid ethyl ester

Intermediate 43 (1.4 g, 2.6 mmol) and ammonium acetate (2.0 g, 26 mmol) were dissolved in MeCN (25 mL) and heated at reflux for 4 h. The reaction mixture was cooled, 10% aqueous NaOH was added and the reaction mixture was extracted with DCM (3×100 mL). The combined organics were further washed with NaHCO₃ and brine and then the organic extracts were dried (Na₂SO₄) and concentrated in vacuo. The crude residue was triturated (1:1) with MeCN and isopropyl ether, and the precipitate was filtered off and dried to give the title compound as a cream solid (550 mg, 42%). δ_{H (}DMSO-d6) 10.04 (1H, br s), 8.85 (1H, br s), 7.87 (1H, dd, J 10.0, 1.8 Hz), 7.71 (1H, dd, J 8.4, 1.1 Hz), 7.41 (1H, t, J 8.5 Hz), 4.36 (2H, q, J 7.1 Hz), 2.91 (4H, d, J 4.2 Hz), 1.34 (3H, t, J 7.1 Hz), 1.02 (6H, s). One exchangeable proton was not observed. LCMS (ES⁺) RT 2.46 minutes, 502 (M+H)⁺.

### Example 208

### 2-(2-Fluoro-4-iodophenylamino)-8-imino-5,5-dimethyl-5,6,7,8-tetrahydro-4H-thieno[2,3-c]azepine-3-carboxylic acid (2-amino-2-methylpropyl)amide

Trimethylaluminium (1.72 mL, 3.45 mmol) was added to a solution of 1,2-diamino-2-methylpropane (303 mg, 3.45 mmol) in THF (10 mL) at 0° C. and stirred for 30 minutes. Example 207 was added to the mixture and then heated to 100° C. for 4 h. Aqueous NaOH (10%) was added to the reaction mixture and the aqueous phase extracted with EtOAc (3×100 mL). The combined organic extracts were dried (Na₂SO₄) and concentrated in vacuo. The crude product was purified by trituration with MeCN and filtered off to give the title compound as a yellow solid (149 mg, 40%). δ_{H} (DMSO-d6) 10.52 (1H, t, J 5.6 Hz), 7.49 (1H, dd, J 10.3, 1.9 Hz), 7.41 (1H, d, J 8.5 Hz), 7.12 (1H, t, J 8.7 Hz), 3.23 (2H, s), 3.12 (2H, d, J 5.7 Hz), 2.80 (2H, s), 0.98 (12H, s). LCMS (ES⁺) RT 2.23 minutes, 544 (M+H)⁺.

### Example 209

### 2-(2-Fluoro-4-iodophenylamino)-5,5-dimethyl-7-oxo-4,5,6,7-tetrahydro-benzo[b]thiophene-3-carbonitrile

2-Fluoro-4-iodoaniline (35 mg, 0.15 mmol) was dissolved in THF and the solution cooled to 0° C. Sodium hexamethyldisilazide (0.6M solution in THF, 0.27 ml) was added dropwise to the solution and the reaction was stirred for 10 min. Intermediate 48 (36 mg, 0.135 mmol) was then added and the reaction stirred for a further 3 h. The solvent was then removed under reduced pressure and purified by preparative HPLC to yield the title compound (3 mg, 5%). δ_{H} (d₆-DMSO) 10.79 (1H, s), 7.82-7.75 (1H, m), 7.64-7.58 (1H, m), 7.25 (1H, t, J 8.9 Hz), 2.65 (2H, s), 2.34 (2H, s), 1.06 (6H, s). LCMS (ES⁺) RT (pH 10) 2.63 minutes, 441 (M+H)⁺.

### Example 210

### 2-(2-Fluoro-4-iodophenylamino)-5,5-dimethyl-7-oxo-4,5,6,7-tetrahydro-benzo[b]thiophene-3-carboxylic acid amide

Hydroxylamine (0.5 mL, 50% solution in water) was added to a solution of Example 209 (83 mg, 0.19 mmol) in THF (5 mL) and heated to reflux for 18 h. The reaction was cooled and brine (50 mL) was added to the residue and the mixture extracted with EtOAc (3×50 mL). The combined organic extracts were dried (Na₂SO₄) and concentrated in vacuo. The crude product was purified by chromatography (silica, 0-30% EtOAc in DCM) to give the title compound as a cream solid (13 mg, 23%). δ_{H} (DMSO-d6) 11.33 (1H, br s), 7.74 (1H, d, J 10.4 Hz), 7.62 (1H, d, J 8.4 Hz), 7.44-7.39 (3H, m), 2.93 (2H, s), 2.35 (2H, s), 1.05 (6H, s). LCMS (ES⁺) RT 2.94 minutes, 459 (M+H)⁺.

### Example 211

### Ethyl 2-[(2-fluoro-4-iodophenyl)amino]-5,5-dimethyl-7-oxo-4,5,6,7-tetrahydrothieno[2,3-c]pyridine-3-carboxylate

To a stirred solution of 2-fluoro-4-iodoaniline (294 mg, 1.24 mmol) in anhydrous THF (5 mL) at 0° C. was added a solution of lithium hexamethyldisilazide (1.12 mL, 1.24 mmol, 1.06M in THF). The reaction mixture was allowed to stir at 0° C. for one hour. A solution of Intermediate 57 (258 mg, 0.52 mmol) in anhydrous THF (5 mL) was added to the reaction mixture and stirred at ambient temperature for 18 hours. The reaction was quenched by the addition of saturated brine (100 mL) and extracted with EtOAc (3×100 mL), then the combined organic fractions were dried (Na₂SO₄), filtered and the volatiles removed in vacuo. The crude residue was dissolved in DCM (5 mL) and 4M HCl in 1,4-dioxane (1 mL) added. The mixture was stirred at ambient temperature for 3 hours before being neutralized to pH 7.4 by the addition of 10% aqueous sodium hydroxide solution. The aqueous phase was extracted with DCM (3×100 mL) and the combined organic fractions were dried (Na₂SO₄), filtered and the volatiles removed in vacuo. The crude residue was purified by column chromatography (SiO₂, 0-30% EtOAc in DCM) to give the title compound as a pale pink solid (30 mg, 10%). δ_{H} (DMSO-d6) 10.89 (1H, brs), 7.48-7.40 (2H, m), 7.27 (1H, t, J 8.6 Hz), 5.29 (1H, s), 4.24 (2H, q, J 7.1 Hz), 3.13 (2H, s), 1.31 (6H, s), 1.30 (3H, t, J 7.1 Hz). LCMS (ES⁺) RT 3.60 minutes, 489 (M+H)⁺.

When ranges are used herein, for example, for biological activity, such as binding data, chemical properties, such as chemical formulae, or dosage ranges, all combinations and subcombinations of ranges and specific embodiments therein are intended to be included.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A compound of formula (I), or a pharmaceutically acceptable salt or solvate thereof: 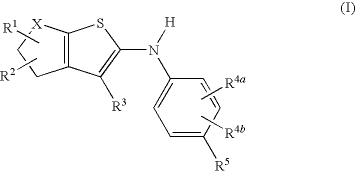
wherein:
-X- is a group of formula (a), (b), (c), (d), (e), (f) or (g): 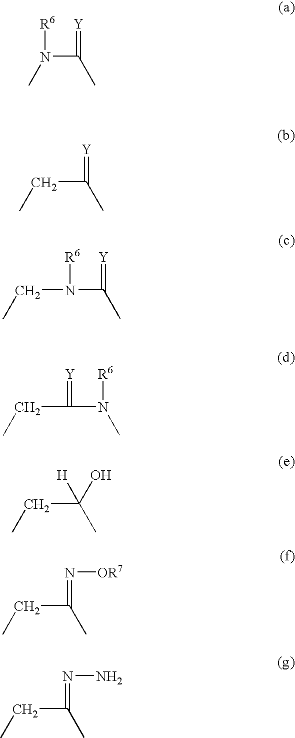
Y is oxygen, sulphur, or N—R⁸;
R¹ and R² are each independently hydrogen, optionally substituted C₁₋₆alkyl, optionally substituted C₃₋₇cycloalkyl, optionally substituted C₃₋₇cycloalkyl(C₁₋₆)alkyl, optionally substituted aryl, optionally substituted aryl(C₁₋₆)alkyl, optionally substituted C₃₋₇heterocycloalkyl, optionally substituted C₃₋₇heterocycloalkyl(C₁₋₆)alkyl, optionally substituted heteroaryl or optionally substituted heteroaryl(C₁₋₆)alkyl; or
R¹ and R², when both are attached to the same carbon atom, are taken together with the carbon atom to which they are attached to form optionally substituted C₃₋₇cycloalkyl or optionally substituted C₃₋₇heterocycloalkyl; or
R¹ and R², when attached to adjacent carbon atoms, are taken together with the carbon atoms through which they are connected to form optionally substituted, optionally benzo-fused C₅₋₇cycloalkyl, optionally substituted, optionally benzo-fused phenyl or optionally substituted, optionally benzo-fused heteroaryl;
R³ is hydrogen, cyano, —CO₂R^{a}, —COR^{b}, —CONR^{b}R^{c}, —SO₂NR^{b}R^{c}, —CON(OR^{b})R^{c}, —CON(R^{c})COR^{b}, —CON(R^{c})SO₂R^{b}, —SO₂N(R^{c})COR^{b}, —CON(R^{d})NR^{b}R^{c}, —C(═NR^{e})NR^{b}R^{c}, —CON(R^{d})C(═NR^{e})NR^{b}R^{c}, C₁₋₆alkyl, C₃₋₇heterocycloalkenyl optionally substituted with one or two methyl groups, optionally substituted furan, optionally substituted thiophene, optionally substituted pyrrole, optionally substituted oxazole, optionally substituted thiazole, optionally substituted isoxazole, optionally substituted isothiazole, optionally substituted imidazole, optionally substituted pyrazole, optionally substituted oxadiazole, optionally substituted thiadiazole, optionally substituted triazole, optionally substituted tetrazole, optionally substituted pyridine, optionally substituted pyrazine, optionally substituted pyrimidine, optionally substituted pyridazine or optionally substituted triazine;
R^{4a} and R^{4b} are each independently hydrogen, halogen, cyano, nitro, C₁₋₆alkyl, trifluoromethyl, C₁₋₆alkoxy, trifluoromethoxy, C₁₋₆alkylthio, C₁₋₆alkylsulphinyl or C₁₋₆alkylsulphonyl;
R⁵ is halogen, nitro, cyano, C₁₋₆alkyl, C₂₋₆alkynyl, hydroxy(C₁₋₆)alkyl or formyl;
R⁶ is hydrogen, C₁₋₆alkyl, formyl, C₂₋₆alkylcarbonyl, trifluoromethylcarbonyl or C₁₋₆ alkylsulphonyl;
R⁷ and R⁸ are each independently hydrogen or C₁₋₆alkyl;
R^{a} is hydrogen, C₁₋₆alkyl or C₃₋₇heterocycloalkyl(C₁₋₆)alkyl;
R^{b} is hydrogen, optionally substituted C₁₋₆alkyl, optionally substituted C₃₋₇cycloalkyl, optionally substituted C₃₋₇cycloalkyl(C₁₋₆)alkyl, optionally substituted aryl, optionally substituted aryl(C₁₋₆)alkyl, optionally substituted C₃₋₇heterocycloalkyl, optionally substituted C₃₋₇heterocycloalkyl(C₁₋₆)alkyl, optionally substituted C₄₋₉heterobicycloalkyl, optionally substituted heteroaryl or optionally substituted heteroaryl(C₁₋₆)alkyl;
R^{c} is hydrogen or C₁₋₆alkyl optionally substituted with hydroxy; or
R^{b} and R^{c} are taken together with the nitrogen atom to which they are both attached to form optionally substituted azetidinyl, optionally substituted pyrrolidinyl, optionally substituted piperidinyl, optionally substituted morpholinyl, optionally substituted thiomorpholinyl, optionally substituted piperazinyl, optionally substituted homopiperidinyl, optionally substituted homomorpholinyl or optionally substituted homopiperazinyl; and
R^{d} and R^{e} are each independently hydrogen or C₁₋₆alkyl.

2. A compound according to claim 1, wherein -X- is a group of formula (a), (b) or (c).

3. A compound according to claim 1, wherein R³ is hydrogen, cyano, —CO₂R^{a}, —CONR^{b}R^{c}, —CON(OR^{b})R^{c}, —CON(R^{d})NR^{b}R^{c}, —C(═NR^{e})NR^{b}R^{c}, —CON(R^{d})C(═NR^{e})NR^{b}R^{c}, C₁₋₆alkyl or C₃₋₇heterocycloalkenyl optionally substituted with one or two methyl groups.

4. A compound according to claim 2, wherein R³ is hydrogen, cyano, —CO₂R^{a}, —CONR^{b}R^{c}, —CON(OR^{b})R^{c}, —CON(R^{d})NR^{b}R^{c}, —C(═NR^{e})NR^{b}R^{c}, —CON(R^{d})C(═NR^{e})NR^{b}R^{c}, C₁₋₆alkyl or C₃₋₇heterocycloalkenyl optionally substituted with one or two methyl groups.

5. A compound according to claim 1, wherein R^{b} is hydrogen; optionally substituted C₁₋₆alkyl, optionally substituted C₃₋₇cycloalkyl(C₁₋₆)alkyl, optionally substituted aryl(C₁₋₆)alkyl, optionally substituted C₃₋₇heterocycloalkyl, optionally substituted C₃₋₇heterocycloalkyl(C₁₋₆)alkyl, optionally substituted C₄₋₉heterobicycloalkyl, optionally substituted heteroaryl or optionally substituted heteroaryl(C₁₋₆)alkyl.

6. A compound according to claim 1, having formula (IIA) or a pharmaceutically acceptable salt or solvate thereof: 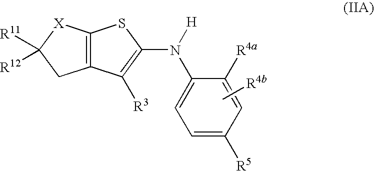
wherein:
R¹¹ is hydrogen or optionally substituted C₁₋₆alkyl; and
R¹² is hydrogen, optionally substituted C₁₋₆alkyl, optionally substituted C₃₋₇cycloalkyl, optionally substituted C₃₋₇cycloalkyl(C₁₋₆)alkyl, optionally substituted aryl, optionally substituted aryl(C₁₋₆)alkyl, optionally substituted C₃₋₇heterocycloalkyl, optionally substituted C₃₋₇heterocycloalkyl(C₁₋₆)alkyl, optionally substituted heteroaryl or optionally substituted heteroaryl(C₁₋₆)alkyl; or
R¹¹ and R¹² are taken together with the carbon atom to which they are both attached to form optionally substituted C₃₋₇cycloalkyl or optionally substituted C₃₋₇heterocycloalkyl.

7. A compound according to claim 6, having formula (IIB) or a pharmaceutically acceptable salt or solvate thereof: 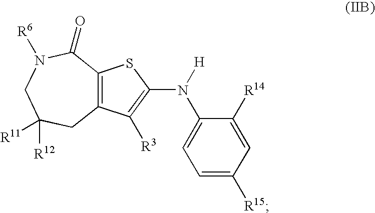
wherein
R¹⁴ is halogen; and
R¹⁵ is halogen, nitro, cyano, C₂₋₆alkynyl, hydroxy(C₁₋₆)alkyl or formyl.

8. A compound according to claim 6, having formula (IIC) or a pharmaceutically acceptable salt or solvate thereof: 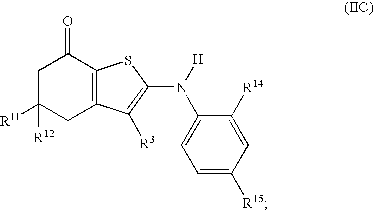
wherein:
R¹⁴ is halogen; and
R¹⁵ is halogen, nitro, cyano, C₂₋₆alkynyl, hydroxy(C₁₋₆)alkyl or formyl.

9. A compound according to claim 6, having formula (IID) or a pharmaceutically acceptable salt or solvate thereof: 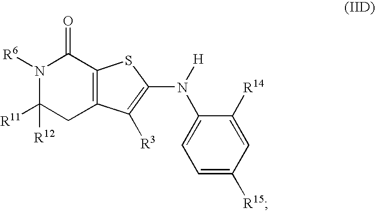
wherein
R¹⁴ is halogen; and
R¹⁵ is halogen, nitro, cyano, C₂₋₆alkynyl, hydroxy(C₁₋₆)alkyl or formyl.

10. A compound according to claim 7, wherein R¹⁴ is fluoro or chloro.

11. A compound according to claim 8, wherein R¹⁴ is fluoro or chloro.

12. A compound according to claim 9, wherein R¹⁴ is fluoro or chloro.

13. A compound according to claim 7, wherein R¹⁵ is iodo.

14. A compound according to claim 8, wherein R¹⁵ is iodo.

15. A compound according to claim 9, wherein R¹⁵ is iodo.

16. A compound according to claim 10, wherein R¹⁵ is iodo.

17. A compound according to claim 10, wherein R¹⁵ is iodo.

18. A compound according to claim 12, wherein R¹⁵ is iodo.

19. A compound or a pharmaceutically acceptable salt or solvate thereof according to claim 1 which is selected from the group consisting of:
2-(2-fluoro-4-iodophenylamino)-5,5-dimethyl-8-oxo-5,6,7,8-tetrahydro-4H-thieno[2,3-c]azepine-3-carboxylic acid ethyl ester;
2-(2-fluoro-4-iodophenylamino)-5,5-dimethyl-8-oxo-5,6,7,8-tetrahydro-4H-thieno[2,3-c]azepine-3-carboxylic acid;
2-(2-fluoro-4-nitrophenylamino)-5,5-dimethyl-8-oxo-5,6,7,8-tetrahydro-4H-thieno[2,3-c]azepine-3-carboxylic acid ethyl ester;
2-(2-chloro-4-iodophenylamino)-5,5-dimethyl-8-oxo-5,6,7,8-tetrahydro-4H-thieno[2,3-c]azepine-3-carboxylic acid ethyl ester;
2-(2-chloro-4-iodophenylamino)-5,5-dimethyl-8-oxo-5,6,7,8-tetrahydro-4H-thieno[2,3-c]azepine-3-carboxylic acid;
2-(2-chloro-4-iodophenylamino)-5,5-dimethyl-8-oxo-5,6,7,8-tetrahydro-4H-thieno[2,3-c]azepine-3-carboxylic acid (2(S),3-dihydroxypropyl)amide;
2-(2-chloro-4-iodophenylamino)-5,5-dimethyl-3-(piperazin-1-ylcarbonyl)-4,5,6,7-tetrahydrothieno[2,3-c]azepin-8-one hydrochloride;
2-(2-chloro-4-iodophenylamino)-5,5-dimethyl-8-oxo-5,6,7,8-tetrahydro-4H-thieno[2,3-c]azepine-3-carboxylic acid methylamide;
2-(2-chloro-4-iodophenylamino)-5,5-dimethyl-3-(pyrrolidin-1-ylcarbonyl)-4,5,6,7-tetrahydrothieno[2,3-c]azepin-8-one;
2-(2-chloro-4-iodophenylamino)-5,5-dimethyl-3-(4-methylpiperazin-1-ylcarbonyl)-4,5,6,7-tetrahydrothieno[2,3-c]azepin-8-one;
2-(2-chloro-4-iodophenylamino)-5,5-dimethyl-8-oxo-5,6,7,8-tetrahydro-4H-thieno[2,3-c]azepine-3-carboxylic acid N-methoxy-N-methylamide;
2-(2-chloro-4-iodophenylamino)-5,5-dimethyl-8-oxo-5,6,7,8-tetrahydro-4H-thieno[2,3-c]azepine-3-carboxylic acid [2-(pyridin-3-yl)ethyl]amide;
2-(2-chloro-4-iodophenylamino)-5,5-dimethyl-8-oxo-5,6,7,8-tetrahydro-4H-thieno[2,3-c]azepine-3-carboxylic acid [2-(pyridin-4-yl)ethyl]amide;
2-(2-chloro-4-iodophenylamino)-5,5-dimethyl-8-oxo-5,6,7,8-tetrahydro-4H-thieno[2,3-c]azepine-3-carboxylic acid (2-amino-2-methylpropyl)amide;
2-(2-chloro-4-iodophenylamino)-5,5-dimethyl-8-oxo-5,6,7,8-tetrahydro-4H-thieno[2,3-c]azepine-3-carboxylic acid (2(R),3-dihydroxypropoxy)amide;
2-(2-chloro-4-iodophenylamino)-5,5-dimethyl-8-oxo-5,6,7,8-tetrahydro-4H-thieno[2,3-c]azepine-3-carboxylic acid (3 (S),4-dihydroxybutyl)amide;
2-(2-chloro-4-iodophenylamino)-5,5-dimethyl-8-oxo-5,6,7,8-tetrahydro-4H-thieno[2,3-c]azepine-3-carboxylic acid (piperidin-4-ylmethyl)amide;
(S)-2-(2-chloro-4-iodophenylamino)-5,5,7-trimethyl-8-oxo-5,6,7,8-tetrahydro-4H-thieno[2,3-c]azepine-3-carboxylic acid (2,3-dihydroxypropyl)amide;
2-(2-fluoro-4-iodophenylamino)-5,5-dimethyl-8-oxo-5,6,7,8-tetrahydro-4H-thieno[2,3-c]azepine-3-carboxylic acid (2-hydroxy-2-methylpropyl)amide;
2-(2-fluoro-4-iodophenylamino)-5,5-dimethyl-8-oxo-5,6,7,8-tetrahydro-4H-thieno[2,3-c]azepine-3-carboxylic acid (2-amino-2-methylpropyl)amide;
2-(2-fluoro-4-iodophenylamino)-5,5-dimethyl-8-oxo-5,6,7,8-tetrahydro-4H-thieno[2,3-c]azepine-3-carboxylic acid (1,1-dimethyl-2-hydroxyethyl)amide;
2-(2-fluoro-4-iodophenylamino)-5,5-dimethyl-8-oxo-5,6,7,8-tetrahydro-4H-thieno[2,3-c]azepine-3-carboxylic acid (3-aminopropyl)amide;
(R)-2-(2-fluoro-4-iodophenylamino)-5,5-dimethyl-8-oxo-5,6,7,8-tetrahydro-4H-thieno[2,3-c]azepine-3-carboxylic acid (piperidin-3-yl)amide hydrochloride;
(S)-2-(2-fluoro-4-iodophenylamino)-5,5-dimethyl-8-oxo-5,6,7,8-tetrahydro-4H-thieno[2,3-c]azepine-3-carboxylic acid (piperidin-3-yl)amide hydrochloride;
2-(2-fluoro-4-iodophenylamino)-5,5-dimethyl-8-oxo-5,6,7,8-tetrahydro-4H-thieno[2,3-c]azepine-3-carboxylic acid (3-amino-2-hydroxypropyl)amide;
2-(2-fluoro-4-iodophenylamino)-5,5-dimethyl-8-oxo-5,6,7,8-tetrahydro-4H-thieno[2,3-c]azepine-3-carboxylic acid (2-aminoethyl)amide hydrochloride;
2-(2-fluoro-4-iodophenylamino)-5,5-dimethyl-8-oxo-5,6,7,8-tetrahydro-4H-thieno[2,3-c]azepine-3-carboxylic acid (2-hydroxypropyl)amide;
2-(2-fluoro-4-iodophenylamino)-3-(3-hydroxyazetidin-1-ylcarbonyl)-5,5-dimethyl-4,5,6,7-tetrahydrothieno[2,3-c]azepin-8-one;
2-(2-fluoro-4-formylphenylamino)-5,5-dimethyl-8-oxo-5,6,7,8-tetrahydro-4H-thieno[2,3-c]azepine-3-carboxylic acid ethyl ester;
2-[2-fluoro-4-(hydroxymethyl)phenylamino]-5,5-dimethyl-8-oxo-5,6,7,8-tetrahydro-4H-thieno[2,3-c]azepine-3-carboxylic acid ethyl ester;
2-(2-fluoro-4-iodophenylamino)-5,5-dimethyl-8-oxo-5,6,7,8-tetrahydro-4H-thieno[2,3-c]azepine-3-carboxylic acid hydrazide;
2-(2-fluoro-4-iodophenylamino)-5,5-dimethyl-8-oxo-5,6,7,8-tetrahydro-4H-thieno[2,3-c]azepine-3-carboxylic acid [2-(3H-imidazol-4-yl)ethyl]amide;
(S)-2-(2-fluoro-4-iodophenylamino)-5,5-dimethyl-8-oxo-5,6,7,8-tetrahydro-4H-thieno[2,3-c]azepine-3-carboxylic acid (2,3-dihydroxypropyl)amide;
2-(2-fluoro-4-iodophenylamino)-5,5-dimethyl-8-oxo-5,6,7,8-tetrahydro-4H-thieno[2,3-c]azepine-3-carboxylic acid (1-methylpiperidin-4-yl)amide;
(S)-2-(2-fluoro-4-iodophenylamino)-3-(3-hydroxypyrrolidin-1-ylcarbonyl)-5,5-dimethyl-4,5,6,7-tetrahydrothieno[2,3-c]azepin-8-one;
(S)-2-(2-fluoro-4-iodophenylamino)-3-[2-(hydroxymethyl)pyrrolidin-1-ylcarbonyl]-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid (3-hydroxypropyl)amide;
(R)-2-(2-fluoro-4-iodophenylamino)-5,5-dimethyl-8-oxo-5,6,7,8-tetrahydro-4H-thieno[2,3-c]azepine-3-carboxylic acid (2,3-dihydroxypropoxy)amide;
2-(2-fluoro-4-iodophenylamino)-5,5-dimethyl-8-oxo-5,6,7,8-tetrahydro-4H-thieno[2,3-c]azepine-3-carboxylic acid (4-aminobutyl)amide;
2-(2-chloro-4-iodophenylamino)-8-imino-5,5-dimethyl-5,6,7,8-tetrahydro-4H-thieno[2,3-c]azepine-3-carboxylic acid amide;
2-(2-chloro-4-iodophenylamino)-8-imino-5,5-dimethyl-5,6,7,8-tetrahydro-4H-thieno[2,3-c]azepine-3-carboxylic acid ethyl ester;
2-(2-fluoro-4-iodophenylamino)-5,5-dimethyl-4,5,6,7-tetrahydrothieno[2,3-c]azepin-8-one;
2-(2-chloro-4-ethynylphenylamino)-5,5-dimethyl-8-oxo-5,6,7,8-tetrahydro-4H-thieno[2,3-c]azepine-3-carboxylic acid (2S,3-dihydroxypropyl)amide;
2-(2-fluoro-4-iodophenylamino)-5,5-dimethyl-8-oxo-5,6,7,8-tetrahydro-4H-thieno[2,3-c]azepine-3-carboxylic acid (2-hydroxypropyl)amide;
2-(2-fluoro-4-iodophenylamino)-5,5-dimethyl-3-(morpholin-4-ylcarbonyl)-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid N′-(pyridin-2-yl)hydrazide;
2-(2-fluoro-4-iodophenylamino)-3-[4-(2-hydroxyethyl)piperazin-1-ylcarbonyl]-5,5-dimethyl-4,5,6,7-tetrahydrothieno[2,3-c]azepin-8-one;
3-{[2-(2-fluoro-4-iodophenylamino)-5,5-dimethyl-8-oxo-5,6,7,8-tetrahydro-4H-thieno[2,3-c]azepin-3-ylcarbonyl]amino}propionic acid ethyl ester;
{1-[2-(2-fluoro-4-iodophenylamino)-5,5-dimethyl-8-oxo-5,6,7,8-tetrahydro-4H-thieno[2,3-c]azepin-3-ylcarbonyl]azetidin-3-ylmethyl}carbamic acid tert-butyl ester;
3(R)-3-{[2-(2-fluoro-4-iodophenylamino)-5,5-dimethyl-8-oxo-5,6,7,8-tetrahydro-4H-thieno[2,3-c]azepin-3-ylcarbonyl]amino}pyrrolidine-1-carboxylic acid tert-butyl ester;
3-([1,4]diazepan-1-ylcarbonyl)-2-(2-fluoro-4-iodophenylamino)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid 2-(dimethylamino)ethylamide;
2-(2-fluoro-4-iodophenylamino)-5,5-dimethyl-8-oxo-5,6,7,8-tetrahydro-4H-thieno[2,3-c]azepine-3-carboxylic acid 2-(morpholin-4-yl)ethylamide;
2-(2-fluoro-4-iodophenylamino)-5,5-dimethyl-8-oxo-5,6,7,8-tetrahydro-4H-thieno[2,3-c]azepine-3-carboxylic acid 3-(dimethylamino)-2,2-dimethylpropylamide;
2-(2-fluoro-4-iodo-phenylamino)-5,5-dimethyl-8-oxo-5,6,7,8-tetrahydro-4H-thieno[2,3-c]azepine-3-carboxylic acid 3-(4-methylpiperazin-1-yl)propylamide;
2-(2-fluoro-4-iodophenylamino)-5,5-dimethyl-8-oxo-5,6,7,8-tetrahydro-4H-thieno[2,3-c]azepine-3-carboxylic acid [(2R)-pyrrolidin-2-ylmethyl]amide;
2-(2-fluoro-4-iodophenylamino)-5,5-dimethyl-8-oxo-5,6,7,8-tetrahydro-4H-thieno[2,3-c]azepine-3-carboxylic acid [2(S)-pyrrolidin-2-ylmethyl]amide;
2-(2-fluoro-4-iodophenylamino)-5,5-dimethyl-3-(4-methylpiperazin-1-ylcarbonyl)-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid [3(S)-pyrrolidin-3-yl]amide hydrochloride;
2-(2-fluoro-4-iodophenylamino)-5,5-dimethyl-8-oxo-5,6,7,8-tetrahydro-4H-thieno[2,3-c]azepine-3-carboxylic acid [3(R)-pyrrolidin-3-yl]amide hydrochloride;
3-({[2-(2-fluoro-4-iodophenylamino)-5,5-dimethyl-8-oxo-5,6,7,8-tetrahydro-4H-thieno[2,3-c]azepin-3-ylcarbonyl]amino}methyl)azetidine-1-carboxylic acid tert-butyl ester;
2-(2-fluoro-4-iodophenylamino)-5,5-dimethyl-8-oxo-5,6,7,8-tetrahydro-4H-thieno[2,3-c]azepine-3-carboxylic acid (azetidin-3-ylmethyl)amide;
3-[3-(aminomethyl)azetidin-1-ylcarbonyl]-2-(2-fluoro-4-iodophenylamino)-5,5-dimethyl-4,5,6,7-tetrahydrothieno[2,3-c]azepin-8-one;
(+)-2-({[2-(2-fluoro-4-iodophenylamino)-5,5-dimethyl-8-oxo-5,6,7,8-tetrahydro-4H-thieno[2,3-c]azepin-3-ylcarbonyl]amino}methyl)piperidine-1-carboxylic acid tert-butyl ester;
N-(2-{[2-(2-fluoro-4-iodophenylamino)-5,5-dimethyl-8-oxo-5,6,7,8-tetrahydro-4H-thieno[2,3-c]azepin-3-ylcarbonyl]amino}ethyl)-N-methylcarbamic acid tert-butyl ester;
2-(2-fluoro-4-iodophenylamino)-5,5-dimethyl-8-oxo-5,6,7,8-tetrahydro-4H-thieno[2,3-c]azepine-3-carboxylic acid 2-(methylamino)ethylamide hydrochloride;
2-(2-fluoro-4-iodophenylamino)-5,5-dimethyl-8-oxo-5,6,7,8-tetrahydro-4H-thieno[2,3-c]azepine-3-carboxylic acid (piperidin-2-ylmethyl)amide hydrochloride;
2-(2-fluoro-4-iodophenylamino)-5,5-dimethyl-8-oxo-5,6,7,8-tetrahydro-4H-thieno[2,3-c]azepine-3-carboxylic acid [3(R)-1-methylpiperidin-3-yl]amide;
3-(3-aminoazetidin-1-ylcarbonyl)-2-(2-fluoro-4-iodophenylamino)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid azetidin-3-ylamide;
3-[3-(aminomethyl)azetidin-1-ylcarbonyl]-2-(2-fluoro-4-iodophenylamino)-5,5,7-trimethyl-4,5,6,7-tetrahydrothieno[2,3-c]azepin-8-one;
2-(2-fluoro-4-iodophenylamino)-3-[3-hydroxy-3-(nitromethyl)azetidin-1-ylcarbonyl]-5,5-dimethyl-4,5,6,7-tetrahydrothieno[2,3-c]azepin-8-one;
3-[3-(aminomethyl)-3-hydroxyazetidin-1-ylcarbonyl]-2-(2-fluoro-4-iodophenylamino)-5,5-dimethyl-4,5,6,7-tetrahydrothieno[2,3-c]azepin-8-one;
3-({[2-(2-fluoro-4-iodophenylamino)-5,5-dimethyl-8-oxo-5,6,7,8-tetrahydro-4H-thieno[2,3-c]azepin-3-ylcarbonyl]amino}methyl)-3-hydroxyazetidine-1-carboxylic acid tert-butyl ester;
2-(2-fluoro-4-iodophenylamino)-5,5-dimethyl-8-oxo-5,6,7,8-tetrahydro-4H-thieno[2,3-c]azepine-3-carboxylic acid (3-hydroxyazetidin-3-ylmethyl)amide;
2-(2-chloro-4-iodophenylamino)-5,5-dimethyl-8-oxo-5,6,7,8-tetrahydro-4H-thieno[2,3-c]azepine-3-carbonitrile;
2-(2-chloro-4-cyanophenylamino)-5,5-dimethyl-8-oxo-5,6,7,8-tetrahydro-4H-thieno[2,3-c]azepine-3-carboxylic acid ethyl ester;
2-(2-chloro-4-iodophenylamino)-5,5-dimethyl-8-oxo-N-propyl-5,6,7,8-tetrahydro-4H-thieno[2,3-c]azepine-3-carboxamidine;
2-(2-chloro-4-iodophenylamino)-3-(4,4-dimethyl-4,5-dihydro-1H-imidazol-2-yl)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid (2-hydroxyethoxy)amide;
2-(2-chloro-4-iodophenylamino)-N-(2,3-dihydroxypropyl)-5,5-dimethyl-8-oxo-5,6,7,8-tetrahydro-4H-thieno[2,3-c]azepine-3-carboxamidine;
2-(2-chloro-4-iodophenylamino)-5,5-dimethyl-8-oxo-5,6,7,8-tetrahydro-4H-thieno[2,3-c]azepine-3-carboxylic acid amide;
2-(2-fluoro-4-iodophenylamino)-5,5-dimethyl-8-oxo-5,6,7,8-tetrahydro-4H-thieno[2,3-c]azepine-3-carboxylic acid (2-aminoethoxy)amide;
N-[2-(2-fluoro-4-iodophenylamino)-5,5-dimethyl-8-oxo-5,6,7,8-tetrahydro-4H-thieno[2,3-c]azepin-3-ylcarbonyl]guanidine;
2-(2-fluoro-4-iodophenylamino)-5,5,7-trimethyl-8-oxo-5,6,7,8-tetrahydro-4H-thieno[2,3-c]azepine-3-carboxylic acid pyrrolidin-3-ylamide;
2-(2-fluoro-4-iodophenylamino)-5,5,7-trimethyl-8-oxo-5,6,7,8-tetrahydro-4H-thieno[2,3-c]azepine-3-carboxylic acid (2-amino-2-methylpropyl)amide;
2-(2-fluoro-4-iodophenylamino)-5,5-dimethyl-8-oxo-5,6,7,8-tetrahydro-4H-thieno[2,3-c]azepine-3-carboxylic acid (6-aminopyridin-3-yl)amide;
2-(2-fluoro-4-iodophenylamino)-5,5-dimethyl-8-oxo-5,6,7,8-tetrahydro-4H-thieno[2,3-c]azepine-3-carboxylic acid (6-aminopyridin-2-yl)amide;
2-(2-fluoro-4-iodophenylamino)-5,5-dimethyl-8-oxo-5,6,7,8-tetrahydro-4H-thieno[2,3-c]azepine-3-carboxylic acid N-(2,3-dihydroxypropyl)-N-methylamide;
2-(2-fluoro-4-iodophenylamino)-3,5,5-trimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid amide;
2-(2-fluoro-4-iodophenylamino)-5,5-dimethyl-8-oxo-5,6,7,8-tetrahydro-4H-thieno[2,3-c]azepine-3-carboxylic acid bis(2-hydroxyethyl)amide;
2-(2-fluoro-4-iodophenylamino)-3-[2-(hydroxymethyl)piperidin-1-ylcarbonyl]-5,5-dimethyl-4,5,6,7-tetrahydrothieno[2,3-c]azepin-8-one;
(R)-{1-[2-(2-fluoro-4-iodophenylamino)-5,5-dimethyl-8-oxo-5,6,7,8-tetrahydro-4H-thieno[2,3-c]azepin-3-ylcarbonyl]pyrrolidin-3-yl}carbamic acid tert-butyl ester;
(S)-{1-[2-(2-fluoro-4-iodophenylamino)-5,5-dimethyl-8-oxo-5,6,7,8-tetrahydro-4H-thieno[2,3-c]azepin-3-ylcarbonyl]pyrrolidin-3-yl}carbamic acid tert-butyl ester;
(R)-{1-[2-(2-fluoro-4-iodophenylamino)-5,5-dimethyl-8-oxo-5,6,7,8-tetrahydro-4H-thieno[2,3-c]azepin-3-ylcarbonyl]pyrrolidin-2-ylmethyl}carbamic acid tert-butyl ester;
(S)-{1-[2-(2-fluoro-4-iodophenylamino)-5,5-dimethyl-8-oxo-5,6,7,8-tetrahydro-4H-thieno[2,3-c]azepin-3-ylcarbonyl]pyrrolidin-2-ylmethyl}carbamic acid tert-butyl ester;
3-[3(R)-3-aminopyrrolidin-1-ylcarbonyl]-2-(2-fluoro-4-iodophenylamino)-5,5-dimethyl-4,5,6,7-tetrahydrothieno[2,3-c]azepin-8-one hydrochloride;
3-[3(S)-3-aminopyrrolidin-1-ylcarbonyl)-2-(2-fluoro-4-iodophenylamino)-5,5-dimethyl-4,5,6,7-tetrahydrothieno[2,3-c]azepin-8-one hydrochloride;
3-[2(R)-2-(aminomethyl)pyrrolidin-1-ylcarbonyl)-2-(2-fluoro-4-iodophenylamino)-5,5-dimethyl-4,5,6,7-tetrahydrothieno[2,3-c]azepin-8-one hydrochloride;
3-[2(S)-2-(aminomethyl)pyrrolidin-1-ylcarbonyl)-2-(2-fluoro-4-iodophenylamino)-5,5-dimethyl-4,5,6,7-tetrahydrothieno[2,3-c]azepin-8-one hydrochloride;
2-(2-fluoro-4-iodophenylamino)-5,5-dimethyl-8-oxo-5,6,7,8-tetrahydro-4H-thieno[2,3-c]azepine-3-carboxylic acid (1-methylpyrrolidin-3-yl)amide;
2(S)-2-tert-butoxycarbonylamino-3-{[2-(2-fluoro-4-iodophenylamino)-5,5-dimethyl-8-oxo-5,6,7,8-tetrahydro-4H-thieno[2,3-c]azepin-3-ylcarbonyl]amino}propionic acid;
2(S)-2-amino-3-{[2-(2-fluoro-4-iodophenylamino)-5,5-dimethyl-8-oxo-5,6,7,8-tetrahydro-4H-thieno[2,3-c]azepin-3-ylcarbonyl]amino}propionic acid hydrochloride;
2-(2-fluoro-4-iodophenylamino)-3-[3(S)-3-(hydroxymethyl)morpholin-4-ylcarbonyl]-5,5-dimethyl-4,5,6,7-tetrahydrothieno[2,3-c]azepin-8-one;
(N-{ethoxycarbonylmethyl}-N-{3(R)-1-[2-(2-fluoro-4-iodophenylamino)-5,5-dimethyl-8-oxo-5,6,7,8-tetrahydro-4H-thieno[2,3-c]azepin-3-ylcarbonyl]pyrrolidin-3-yl}amino)acetic acid ethyl ester;
2-(2-fluoro-4-iodophenylamino)-5,5-dimethyl-8-oxo-5,6,7,8-tetrahydro-4H-thieno[2,3-c]azepine-3-carboxylic acid piperidin-2(R)-ylmethyl ester;
2-(2-fluoro-4-iodophenylamino)-5,5-dimethyl-8-oxo-5,6,7,8-tetrahydro-4H-thieno[2,3-c]azepine-3-carboxylic acid piperidin-2(S)-ylmethyl ester;
(S)-4-[2-(2-fluoro-4-iodophenylamino)-5,5-dimethyl-8-oxo-5,6,7,8-tetrahydro-4H-thieno[2,3-c]azepin-3-ylcarbonyl]piperazine-1,3-dicarboxylic acid 1-tert-butyl ester triethylamine salt;
(R)-4-[2-(2-fluoro-4-iodophenylamino)-5,5-dimethyl-8-oxo-5,6,7,8-tetrahydro-4H-thieno[2,3-c]azepin-3-ylcarbonyl]piperazine-1,3-dicarboxylic acid 1-tert-butyl ester triethylamine salt;
1-[2-(2-fluoro-4-iodophenylamino)-5,5-dimethyl-8-oxo-5,6,7,8-tetrahydro-4H-thieno[2,3-c]azepin-3-ylcarbonyl]piperazine-2(S)-carboxylic acid hydrochloride salt;
1-[2-(2-fluoro-4-iodophenylamino)-5,5-dimethyl-8-oxo-5,6,7,8-tetrahydro-4H-thieno[2,3-c]azepin-3-ylcarbonyl]piperazine-2(R)-carboxylic acid hydrochloride salt;
2-(2-fluoro-4-iodophenylamino)-5,5-dimethyl-8-oxo-5,6,7,8-tetrahydro-4H-thieno[2,3-c]azepine-3-carboxylic acid N-methyl-N-[2-(methylamino)ethyl]amide;
2-(2-fluoro-4-iodophenylamino)-5,5-dimethyl-8-oxo-5,6,7,8-tetrahydro-4H-thieno[2,3-c]azepine-3-carboxylic acid (2-hydroxyethyl)amide;
2-(2-fluoro-4-iodophenylamino)-3-(4-hydroxypiperidin-1-ylcarbonyl)-5,5-dimethyl-4,5,6,7-tetrahydrothieno[2,3-c]azepin-8-one;
2-(2-fluoro-4-iodophenylamino)-3-(3-hydroxypyrrolidin-1-ylcarbonyl)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid (3-hydroxypropyl)amide;
2-(2-fluoro-4-iodophenylamino)-5,5-dimethyl-8-oxo-5,6,7,8-tetrahydro-4H-thieno[2,3-c]azepine-3-carboxylic acid (2-hydroxypropyl)amide;
2-(2-fluoro-4-iodophenylamino)-5,5-dimethyl-8-oxo-5,6,7,8-tetrahydro-4H-thieno[2,3-c]azepine-3-carboxylic acid [3-(2-oxopyrrolidin-1-yl)propyl]amide;
2-(2-fluoro-4-iodophenylamino)-3-[4-(2-hydroxyethyl)piperazin-1-ylcarbonyl]-5,5-dimethyl-4,5,6,7-tetrahydrothieno[2,3-c]azepin-8-one;
3-{[2-(2-fluoro-4-iodophenylamino)-5,5-dimethyl-8-oxo-5,6,7,8-tetrahydro-4H-thieno[2,3-c]azepin-3-ylcarbonyl]amino}propionic acid ethyl ester;
2-(2-fluoro-4-iodophenylamino)-5,5-dimethyl-8-oxo-5,6,7,8-tetrahydro-4H-thieno[2,3-c]azepine-3-carboxylic acid [2-(dimethylamino)ethyl]amide;
2-(2-fluoro-4-iodophenylamino)-5,5-dimethyl-8-oxo-5,6,7,8-tetrahydro-4H-thieno[2,3-c]azepine-3-carboxylic acid (1-hydroxycyclohexylmethyl)amide;
2-(2-fluoro-4-iodophenylamino)-5,5-dimethyl-8-oxo-5,6,7,8-tetrahydro-4H-thieno[2,3-c]azepine-3-carboxylic acid [3-(dimethylamino)-2,2-dimethylpropyl)amide;
2-(2-fluoro-4-iodophenylamino)-5,5-dimethyl-8-oxo-5,6,7,8-tetrahydro-4H-thieno[2,3-c]azepine-3-carboxylic acid [2-(1-methylpyrrolidin-2-yl)ethyl]amide;
2-(2-fluoro-4-iodophenylamino)-5,5-dimethyl-8-oxo-5,6,7,8-tetrahydro-4H-thieno[2,3-c]azepine-3-carboxylic acid (pyridin-2-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid [2-(pyridin-2-yl)ethyl]amide;
2-(2-fluoro-4-iodophenylamino)-5,5-dimethyl-8-oxo-5,6,7,8-tetrahydro-4H-thieno[2,3-c]azepine-3-carboxylic acid (2-methoxy-1-methylethyl)amide;
2-(2-fluoro-4-iodophenylamino)-5,5-dimethyl-8-oxo-5,6,7,8-tetrahydro-4H-thieno[2,3-c]azepine-3-carboxylic acid [2-(piperidin-1-yl)ethyl]amide;
2-(2-fluoro-4-iodophenylamino)-5,5-dimethyl-8-oxo-5,6,7,8-tetrahydro-4H-thieno[2,3-c]azepine-3-carboxylic acid (pyridin-4-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid [2-(pyrrolidin-1-yl)ethyl]amide;
2-(2-fluoro-4-iodophenylamino)-5,5-dimethyl-8-oxo-5,6,7,8-tetrahydro-4H-thieno[2,3-c]azepine-3-carboxylic acid (tetrahydrofuran-2-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid [2-(acetylamino)ethyl]amide;
3-{[2-(2-fluoro-4-iodophenylamino)-5,5-dimethyl-8-oxo-5,6,7,8-tetrahydro-4H-thieno[2,3-c]azepin-3-ylcarbonyl]amino}butyric acid ethyl ester;
2-(2-fluoro-4-iodophenylamino)-5,5-dimethyl-8-oxo-5,6,7,8-tetrahydro-4H-thieno[2,3-c]azepine-3-carboxylic acid (2-methoxyethyl)amide;
1-(2-{[2-(2-fluoro-4-iodophenylamino)-5,5-dimethyl-8-oxo-5,6,7,8-tetrahydro-4H-thieno[2,3-c]azepin-3-ylcarbonyl]amino}-3-methylbutyryl)pyrrolidine-2-carboxylic acid;
2-(2-fluoro-4-iodophenylamino)-5,5-dimethyl-8-oxo-5,6,7,8-tetrahydro-4H-thieno[2,3-c]azepine-3-carboxylic acid 2-methoxybenzylamide;
2-(2-fluoro-4-iodophenylamino)-5,5-dimethyl-8-oxo-5,6,7,8-tetrahydro-4H-thieno[2,3-c]azepine-3-carboxylic acid (3-isopropoxypropyl)amide;
2-(2-fluoro-4-iodophenylamino)-5,5-dimethyl-8-oxo-5,6,7,8-tetrahydro-4H-thieno[2,3-c]azepine-3-carboxylic acid 3-methoxybenzylamide;
2-(2-fluoro-4-iodophenylamino)-5,5-dimethyl-8-oxo-5,6,7,8-tetrahydro-4H-thieno[2,3-c]azepine-3-carboxylic acid (2-hydroxy-2-phenylethyl)amide;
2-(2-fluoro-4-iodophenylamino)-5,5-dimethyl-8-oxo-5,6,7,8-tetrahydro-4H-thieno[2,3-c]azepine-3-carboxylic acid [3-(dimethylamino)propyl]amide;
2-(2-fluoro-4-iodophenylamino)-5,5-dimethyl-8-oxo-5,6,7,8-tetrahydro-4H-thieno[2,3-c]azepine-3-carboxylic acid (3-amino-2,2-dimethylpropyl)amide;
2-(2-fluoro-4-iodophenylamino)-5,5-dimethyl-8-oxo-5,6,7,8-tetrahydro-4H-thieno[2,3-c]azepine-3-carboxylic acid N-methyl-N-[3-(methylamino)propyl]amide;
2-(2-fluoro-4-iodophenylamino)-5,5-dimethyl-8-oxo-5,6,7,8-tetrahydro-4H-thieno[2,3-c]azepine-3-carboxylic acid (2-oxotetrahydrofuran-3-yl)amide;
2-(2-fluoro-4-iodophenylamino)-5,5-dimethyl-8-oxo-5,6,7,8-tetrahydro-4H-thieno[2,3-c]azepine-3-carboxylic acid (2-ethylsulfanylethyl)amide;
2-(2-fluoro-4-iodophenylamino)-5,5-dimethyl-8-oxo-5,6,7,8-tetrahydro-4H-thieno[2,3-c]azepine-3-carboxylic acid [2-(pyridin-2-ylamino)ethyl]amide;
2-(2-fluoro-4-iodophenylamino)-5,5-dimethyl-8-oxo-5,6,7,8-tetrahydro-4H-thieno[2,3-c]azepine-3-carboxylic acid (carbamoylmethyl)amide;
2-(2-fluoro-4-iodophenylamino)-5,5-dimethyl-3-(3-oxopiperazin-1-ylcarbonyl)-4,5,6,7-tetrahydrothieno[2,3-c]azepin-8-one;
3-(4-acetylpiperazin-1-ylcarbonyl)-2-(2-fluoro-4-iodophenylamino)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid (2-oxo-2-phenylethyl)amide;
1-[2-(2-fluoro-4-iodophenylamino)-5,5-dimethyl-8-oxo-5,6,7,8-tetrahydro-4H-thieno[2,3-c]azepin-3-ylcarbonyl]piperidine-4-carboxylic acid amide;
2-(2-fluoro-4-iodophenylamino)-5,5-dimethyl-8-oxo-5,6,7,8-tetrahydro-4H-thieno[2,3-c]azepine-3-carboxylic acid (pyridin-3-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid [2-(pyridin-3-yl)ethyl]amide;
2-(2-fluoro-4-iodophenylamino)-3-[4-(hydroxymethyl)piperidin-1-ylcarbonyl]-5,5-dimethyl-4,5,6,7-tetrahydrothieno[2,3-c]azepin-8-one;
3-(4-ethylpiperazin-1-ylcarbonyl)-2-(2-fluoro-4-iodophenylamino)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid (thiazolidin-4-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid [2-(2-oxoimidazolidin-1-yl)ethyl]amide;
2-(2-fluoro-4-iodophenylamino)-5,5-dimethyl-8-oxo-5,6,7,8-tetrahydro-4H-thieno[2,3-c]azepine-3-carboxylic acid (1-azabicyclo[2.2.2]oct-3-yl)amide;
2-(2-fluoro-4-iodophenylamino)-5,5-dimethyl-8-oxo-5,6,7,8-tetrahydro-4H-thieno[2,3-c]azepine-3-carboxylic acid (1H-benzimidazol-2-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid (1,2,3,4-tetrahydroquinolin-2-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid (3,3,3-trifluoro-2-hydroxypropyl)amide;
2-(2-fluoro-4-iodophenylamino)-3-(3-hydroxypiperidin-1-ylcarbonyl)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid (3-methyl-3H-imidazol-4-ylmethyl)amide;
2-{[2-(2-fluoro-4-iodophenylamino)-5,5-dimethyl-8-oxo-5,6,7,8-tetrahydro-4H-thieno[2,3-c]azepin-3-ylcarbonyl]amino}-3-(1H-indol-3-yl)propionic acid;
2-(2-fluoro-4-iodophenylamino)-5,5-dimethyl-8-oxo-5,6,7,8-tetrahydro-4H-thieno[2,3-c]azepine-3-carboxylic acid [2-([1,2,4]triazol-4-yl)ethyl]amide;
2-{[2-(2-fluoro-4-iodophenylamino)-5,5-dimethyl-8-oxo-5,6,7,8-tetrahydro-4H-thieno[2,3-c]azepin-3-ylcarbonyl]amino}-5-guanidinopentanoic acid;
2-{[2-(2-fluoro-4-iodophenylamino)-5,5-dimethyl-8-oxo-5,6,7,8-tetrahydro-4H-thieno[2,3-c]azepin-3-ylcarbonyl]amino}-3-(4-hydroxyphenyl)propionic acid;
2-{[2-(2-fluoro-4-iodophenylamino)-5,5-dimethyl-8-oxo-5,6,7,8-tetrahydro-4H-thieno[2,3-c]azepin-3-ylcarbonyl]amino}-4-methylsulfanylbutyric acid;
2-(2-fluoro-4-iodophenylamino)-5,5-dimethyl-8-oxo-5,6,7,8-tetrahydro-4H-thieno[2,3-c]azepine-3-carboxylic acid (1-methylpiperidin-2-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid (1,1-dioxotetrahydrothien-3-yl)amide;
2-(2-fluoro-4-iodophenylamino)-5,5-dimethyl-8-oxo-5,6,7,8-tetrahydro-4H-thieno[2,3-c]azepine-3-carboxylic acid (2-methanesulfonylethyl)amide;
{[2-(2-fluoro-4-iodophenylamino)-5,5-dimethyl-8-oxo-5,6,7,8-tetrahydro-4H-thieno[2,3-c]azepin-3-ylcarbonyl]amino}acetic acid;
2-(2-fluoro-4-iodophenylamino)-5,5-dimethyl-8-oxo-5,6,7,8-tetrahydro-4H-thieno[2,3-c]azepine-3-carboxylic acid [2-(3,5-dimethylpyrazol-1-yl)ethyl]amide;
3-(2,6-dimethylmorpholine-4-ylcarbonyl)-2-(2-fluoro-4-iodophenylamino)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid N-(tert-butyl)-N-(2-hydroxyethyl)amide;
2-(2-fluoro-4-iodophenylamino)-3-[2-(hydroxymethyl)piperidin-1-ylcarbonyl]-5,5-dimethyl-4,5,6,7-tetrahydrothieno[2,3-c]azepin-8-one;
2-(2-fluoro-4-iodophenylamino)-3-(3-hydroxypyrrolidin-1-ylcarbonyl)-5,5-dimethyl-4,5,6,7-tetrahydrothieno[2,3-c]azepin-8-one;
2-(2-fluoro-4-iodophenylamino)-3-[2-(2-hydroxyethyl)piperidin-1-ylcarbonyl]-5,5-dimethyl-4,5,6,7-tetrahydrothieno[2,3-c]azepin-8-one;
2-(2-fluoro-4-iodophenylamino)-3-[2-(hydroxymethyl)pyrrolidin-1-ylcarbonyl]-5,5-dimethyl-4,5,6,7-tetrahydrothieno[2,3-c]azepin-8-one;
1-[2-(2-fluoro-4-iodophenylamino)-5,5-dimethyl-8-oxo-5,6,7,8-tetrahydro-4H-thieno[2,3-c]azepin-3-ylcarbonyl]piperidine-3-carboxylic acid amide;
2-(2-fluoro-4-iodophenylamino)-3-[3-(hydroxymethyl)piperidin-1-ylcarbonyl]-5,5-dimethyl-4,5,6,7-tetrahydrothieno[2,3-c]azepin-8-one;
1-[2-(2-fluoro-4-iodophenylamino)-5,5-dimethyl-8-oxo-5,6,7,8-tetrahydro-4H-thieno[2,3-c]azepin-3-ylcarbonyl]pyrrolidine-2-carboxylic acid methyl ester;
2-(2-fluoro-4-iodophenylamino)-5,5-dimethyl-8-oxo-5,6,7,8-tetrahydro-4H-thieno[2,3-c]azepine-3-carboxylic acid [2-(phenylamino)ethyl]amide;
2-(2-fluoro-4-iodophenylamino)-5,5-dimethyl-8-oxo-5,6,7,8-tetrahydro-4H-thieno[2,3-c]azepine-3-carboxylic acid (2-hydroxy-1-phenylethyl)amide;
2-(2-fluoro-4-iodophenylamino)-3-[4-(2-hydroxyethyl)piperidin-1-ylcarbonyl]-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid N-ethyl-N-[3-(ethylamino)propyl]amide;
2-(2-fluoro-4-iodophenylamino)-5,5-dimethyl-8-oxo-5,6,7,8-tetrahydro-4H-thieno[2,3-c]azepine-3-carboxylic acid {2-[bis(2-hydroxyethyl)amino]ethyl}amide;
2-(2-fluoro-4-iodophenylamino)-5,5-dimethyl-8-oxo-5,6,7,8-tetrahydro-4H-thieno[2,3-c]azepine-3-carboxylic acid [4-(aminomethyl)cyclohexylmethyl]amide;
2-(2-fluoro-4-iodophenylamino)-5,5-dimethyl-3-(1,4,7-triazanonan-1-ylcarbonyl)-4,5,6,7-tetrahydrothieno[2,3-c]azepin-8-one;
2-(2-fluoro-4-iodophenylamino)-3-[2-(methoxymethyl)pyrrolidin-1-ylcarbonyl]-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid N-[2-(dimethylamino)ethyl]-N-ethylamide;
2-(2-fluoro-4-iodophenylamino)-5,5-dimethyl-8-oxo-5,6,7,8-tetrahydro-4H-thieno[2,3-c]azepine-3-carboxylic acid [1-(hydroxymethyl)-2,2-dimethylpropyl]amide;
N-{1-[2-(2-fluoro-4-iodophenylamino)-5,5-dimethyl-8-oxo-5,6,7,8-tetrahydro-4H-thieno[2,3-c]azepin-3-ylcarbonyl]pyrrolidin-3-yl}acetamide;
2-(2-fluoro-4-iodophenylamino)-5,5-dimethyl-8-oxo-5,6,7,8-tetrahydro-4H-thieno[2,3-c]azepine-3-carboxylic acid [1-(carbamoyl)ethyl]amide;
2-(2-fluoro-4-iodophenylamino)-5,5-dimethyl-8-oxo-5,6,7,8-tetrahydro-4H-thieno[2,3-c]azepine-3-carboxylic acid (2-oxoazepan-3-yl)amide;
2-(2-fluoro-4-iodophenylamino)-5,5-dimethyl-8-oxo-5,6,7,8-tetrahydro-4H-thieno[2,3-c]azepine-3-carboxylic acid N-(2-cyanoethyl)-N-ethylamide;
2-(2-fluoro-4-iodophenylamino)-5,5-dimethyl-8-oxo-5,6,7,8-tetrahydro-4H-thieno[2,3-c]azepine-3-carboxylic acid N-methyl-N-{2-[2-(methylamino)ethoxy]-ethyl}amide;
3-{[2-(2-fluoro-4-iodophenylamino)-5,5-dimethyl-8-oxo-5,6,7,8-tetrahydro-4H-thieno[2,3-c]azepin-3-ylcarbonyl]amino}propionic acid;
1-[2-(2-fluoro-4-iodophenylamino)-5,5-dimethyl-8-oxo-5,6,7,8-tetrahydro-4H-thieno[2,3-c]azepin-3-ylcarbonyl]piperidine-2-carboxylic acid;
1-[2-(2-fluoro-4-iodophenylamino)-5,5-dimethyl-8-oxo-5,6,7,8-tetrahydro-4H-thieno[2,3-c]azepin-3-ylcarbonyl]pyrrolidine-2-carboxylic acid;
2-(2-fluoro-4-iodophenylamino)-5,5-dimethyl-8-oxo-5,6,7,8-tetrahydro-4H-thieno[2,3-c]azepine-3-carboxylic acid [3-(aminomethyl)cyclohexylmethyl]amide;
2-(2-fluoro-4-iodophenylamino)-5,5-dimethyl-8-oxo-5,6,7,8-tetrahydro-4H-thieno[2,3-c]azepine-3-carboxylic acid (1-ethylpyrrolidin-2-ylmethyl)amide;
2-(2-fluoro-4-iodophenylamino)-5,5-dimethyl-8-oxo-5,6,7,8-tetrahydro-4H-thieno[2,3-c]azepine-3-carboxylic acid [3-(morpholin-4-yl)propyl]amide;
2-(2-fluoro-4-iodophenylamino)-3-(4-hydroxypiperidin-1-ylcarbonyl)-5,5-dimethyl-4,5,6,7-tetrahydrothieno[2,3-c]azepin-8-one;
2-(2-fluoro-4-iodophenylamino)-5,5-dimethyl-8-oxo-5,6,7,8-tetrahydro-4H-thieno[2,3-c]azepine-3-carboxylic acid N-(2-hydroxyethyl)-N-methylamide;
2-(2-fluoro-4-iodophenylamino)-5,5-dimethyl-8-oxo-5,6,7,8-tetrahydro-4H-thieno[2,3-c]azepine-3-carboxylic acid N-(2-hydroxyethyl)-N-propylamide;
2-(2-fluoro-4-iodophenylamino)-8-imino-5,5-dimethyl-5,6,7,8-tetrahydro-4H-thieno[2,3-c]azepine-3-carboxylic acid ethyl ester;
2-(2-fluoro-4-iodophenylamino)-8-imino-5,5-dimethyl-5,6,7,8-tetrahydro-4H-thieno[2,3-c]azepine-3-carboxylic acid (2-amino-2-methylpropyl)amide;
2-(2-fluoro-4-iodophenylamino)-5,5-dimethyl-7-oxo-4,5,6,7-tetrahydro-benzo[b]thiophene-3-carbonitrile;
2-(2-fluoro-4-iodophenylamino)-5,5-dimethyl-7-oxo-4,5,6,7-tetrahydro-benzo[b]thiophene-3-carboxylic acid amide; and
ethyl 2-[(2-fluoro-4-iodophenyl)amino]-5,5-dimethyl-7-oxo-4,5,6,7-tetrahydrothieno[2,3-c]pyridine-3-carboxylate.

20. A pharmaceutical composition comprising a compound or a pharmaceutically acceptable salt or solvate thereof according to claim 1, in combination with a pharmaceutically acceptable carrier.

21. A method for the treatment or prevention of a disorder for which the administration of a selective MEK inhibitor is indicated which comprises administering to a patient in need of such treatment an effective amount of a compound or pharmaceutically acceptable salt or solvate thereof according to claim 1.

22. A method according to claim 21, wherein the disorder is selected from the group consisting of an autoimmune or inflammatory disorder, a cardiovascular disorder, a proliferative disorder, an oncological condition, and pain or a nociceptive disorder.

23. A method according to claim 22, wherein the disorder is an autoimmune or inflammatory disorder.

24. A method according to claim 23, wherein the autoimmune or inflammatory disorder is selected from the group consisting of rheumatoid arthritis, osteoarthritis, multiple sclerosis, asthma, inflammatory bowel disease, psoriasis and transplant rejection.

25. A method according to claim 22, wherein the disorder is a cardiovascular disorder.

26. A method according to claim 25, wherein the cardiovascular disorder is selected from the group consisting of thrombosis, cardiac hypertrophy, hypertension and irregular contractility of the heart.

27. A method according to claim 22, wherein the disorder is a proliferative disorder.

28. A method according to claim 27, wherein the proliferative disorder is restenosis.

29. A method according to claim 22, wherein the disorder is an oncological condition.

30. A method according to claim 29, wherein the oncological condition is selected from the group consisting of leukemia, glioblastoma, lymphoma, melanoma, and human cancers of the liver, bone, skin, brain, pancreas, lung, breast, stomach, colon, rectum, prostate, ovary and cervix.

31. A method according to claim 22, wherein the disorder is pain or a nociceptive disorder.

32. A method according to claim 31, wherein the pain or nociceptive disorder is selected from the group consisting of chronic pain and neuropathic pain.
